(19) United States
(12) Patent Application Publication
(10) Pub. No.: US 2025/0288402 A1
(43) Pub. Date: Sep. 18, 2025

(54) METHODS AND COMPOSITIONS FOR IMPROVING FERTILITY

(71) Applicants: THE UNIVERSITY OF CHICAGO, Chicago, IL (US); TONGJI UNIVERSITY, Shanghai (CN)

(72) Inventors: Chuan HE, Chicago, IL (US); Jiangbo WEI, Chicago, IL (US); Tiebo ZENG, Chicago, IL (US); Shaorong GAO, Shanghai (CN); Yawei GAO, Shanghai (CN)

(21) Appl. No.: 18/862,649

(22) PCT Filed: May 4, 2023
(86) PCT No.: PCT/CN2023/092057

§ 371 Date: Nov. 4, 2024

Related U.S. Application Data
(60) Provisional application No. 63/338,332, filed on May 4, 2022.

(51) Int. Cl. A61D 19/02; A01K 29/00; A61D 19/04; C12N 5/09; C12N 15/11; C12N 15/877; C12Q 1/6809

(52) U.S. Cl. CPC A61D 19/02; A01K 29/005; A61D 19/04; C12N 5/0693; C12N 15/11; C12N 15/877; C12Q 1/6809; C12Q 2600/154

(57) ABSTRACT

N⁶-methyladenosine (m⁶A) is the most abundant internal modification on mammalian messenger RNA (mRNA). It is installed by a writer complex and can be reversed by erasers such as the fat mass and obesity-associated protein FTO. Despite extensive research, the primary physiological substrates of FTO in mammalian tissues and development remain elusive. Aspects disclosed show that FTO mediates m⁶A demethylation of long-interspersed element-1 LINE1 RNA in mouse embryonic stem cells (mESCs), regulating LINE1 RNA abundance and the local chromatin state, which in turn modulates transcription of LINE1-containing genes. FTO-mediated LINE1 RNA m⁶A demethylation also plays regulatory roles in shaping chromatin state and gene expression during mouse oocyte and embryonic development. FTO overexpression is also disclosed to modulate fertility.

This application claims priority to U.S. Provisional Patent Application Ser. No. 63/338,332, filed May 4, 2022, which is incorporated by reference herein in its entirety.

### BACKGROUND OF THE INVENTION

### I. Field of the Invention

This invention relates to the field of biology, medicine, veterinary medicine, and molecular biology.

### II. Background

N⁶-methyladenosine (m⁶A) is the most prevalent mammalian mRNA internal modification, regulated by writer and eraser proteins, impacting transcript fate through reader proteins (1-3). The fat mass and obesity-associated protein (FTO) was the first RNA demethylase shown to remove mRNA m⁶A (4). FTO is known to be involved in mammalian development and human diseases; for example, Fto^{-/-} mice display severe developmental defects (5, 6). Extensive functional characterizations in human cancer cells have shown that FTO can localize to the cell cytoplasm and remove m⁶A from mRNA transcripts that contribute to cancer progression (7-12); however, similar activity was not apparent across mouse and human tissues where FTO tends to exhibit nuclear localization (13). Another form of m⁶A, m⁶Aₘ, enriched at the cap of a portion of mRNA and certain small nuclear RNAs (snRNAs), was also identified as a substrate of FTO (14-16). However, depletion of the cap-m⁶Aₘ methyltransferase PCIF1 only causes mild cellular effects (17-19) and negligible impacts on mouse viability or fertility (20). These discordant findings suggest that the functionally relevant substrates of FTO during mammalian development remain elusive.

Unlike Pcif1 KO, Mettl3 depletion in mice causes early embryonic lethality (21). It was recently found that chromatin-associated regulatory RNAs (carRNAs) can be m⁶A methylated by METTL3, which regulates chromatin state and transcription in mouse embryonic stem cells (mESCs) (22); independent reports confirmed the chromatin regulation role of carRNA m⁶A and further showed notable effects of m⁶A on the expression of endogenous retroviruses (ERVs) and heterochromatin formation (23-25). Thus, it remained to be seen whether a subset of m⁶A-marked carRNAs could be the physiological substrates of FTO and whether FTO-mediated m⁶A demethylation may regulate chromatin state in mammalian tissues and during early development.

The discovery of FTO as an RNA m⁶A demethylase in 2011 opened of a new field of biology to investigate gene expression regulation by dynamic RNA modifications. The inventors have recently made another potentially paradigm-shifting discovery using Fto knockout (KO) mESCs and mouse models. The inventors discovered prevalent m⁶A methylation of non-coding, chromatin-associated regulatory RNAs (carRNAs). The methylation and FTO-mediated demethylation of carRNAs affected transcription by regulating carRNA abundance and nearby chromatin state. On the other hand, the inventors overexpressed FTO in crops like rice and potato and found that FTO overexpression (OE) can boost the crop yield by ˜50%. Although rice and potato are plants of different family, their overgrowth phenotype were the same, indicating that there is a degree of universality to the process. These findings have important implications for crop breeding and food security, and promoted the inventors to think whether FTO OE in mouse, human and animals (such as livestock) can improve reproduction and increase yield in animal husbandry.

It was previously known that transgenic overexpression of FTO in mice leads to obesity, but a ubiquitous overexpression mouse model cannot tell when and where the FTO OE causes the obesity phenotype. Moreover, transgenic OE, which changes genomic DNA sequences, raises concerns about the safety of genetic modifications.

### BRIEF SUMMARY

In general, the current disclosure relates to the discovery that FTO-mediated m⁶A demethylation regulates LINE1 RNA abundance and local chromatin state in mESCs and mouse development. The disclosure also relates to the discovery that transient FTO overexpression (OE) in early embryos, without changing genomic DNA, promotes embryo growth, increases birth weight and adult body weight, and improves zygote implantation rates.

Methods disclosed include but are not limited to methods for demethylating LINE1 RNA in a cell, methods for modifying expression level of a gene in a cell, methods for increasing chromatin accessibility in a cell, methods for modifying development of a germ cell and include 1, 2, 3, 4, or more steps including any of the following: providing to the cell an effective amount of fat mass and obesity-associated protein (FTO) and/or a nucleic acid molecule encoding FTO, measuring methylation in the cell, and culturing and/or incubating the cells in media and/or buffer.

Methods disclosed also include but are not limited to methods for modifying expression level of a gene in a cell, methods for decreasing chromatin accessibility in a cell, methods for modifying development of a germ cell and include 1, 2, 3, 4, or more steps including any of the following: providing to the cell an effective amount of an FTO inhibitor, measuring methylation in the cell, and culturing and/or incubating the cells in media and/or buffer.

Methods disclosed also include methods of increasing zygote implantation in an animal, methods of increasing litter size, methods of improving an in vitro fertilization process, methods of increasing litter size and number in an animal, methods of increasing zygote implantation rate, methods of increasing birth weight in an animal, methods of decreasing pregnancy loss in an animal, and/or methods for decreasing the risk of metabolic diseases and include 1, 2, 3, 4, 5, 6, 7 or more steps including any of the following: implanting one or more fertilized cells into the reproductive tract of the animal, monitoring the animal for pregnancy or implantation rates, measuring hormonal changes, such as HCG levels in the animal, measuring demethylation, including RNA demethylation, in the fertilized cells, and culturing and/or incubating the fertilized cells in media and/or buffer.

Methods for demethylating long-interspersed element-1 (LINE1) RNA in a cell are disclosed herein. The method can comprise providing to the cell an effective amount of fat mass and obesity-associated protein (FTO) and/or a nucleic acid molecule encoding FTO. Also disclosed are methods for modifying expression level of a gene in a cell, where the method can comprise providing to the cell an effective amount of FTO or a nucleic acid molecule encoding FTO. Also disclosed are methods for increasing chromatin accessibility in a cell, where the method comprises providing to the cell an effective amount of FTO or a nucleic acid molecule encoding FTO. Also disclosed are methods for modifying development of a germ cell, where the method comprises providing to the cell an effective amount of FTO or a nucleic acid molecule encoding FTO. The LINE1 RNA can be chromatin-associated regulatory RNA. The cell can be any cell, such as a germ cell, or fertilized cell. The cell can be from any source such as a human, mouse, or livestock animal.

In some aspects, the method comprises providing a nucleic acid encoding for FTO to the cell. The nucleic acid provided to the cell may also comprise a nucleic acid encoding for a Cas nuclease and a guide RNA (gRNA). The nucleic acid encoding for FTO and the nucleic acid encoding for a Cas nuclease and a guide RNA (gRNA) may be on the same nucleic acid molecule or separate nucleic acid molecules. In some aspects, the method comprises providing an FTO protein to the cell. The FTO protein may be provided as a wild-type protein, mutant protein, and/or fusion protein. The fusion protein may be an FTO protein fused with a Cas nuclease. In aspects with a Cas nuclease fusion, the cell may also be provided a gRNA. The Cas nuclease may be a catalytically-inactive Cas13 (dCas13). The gRNA can target any nucleic acid, including a LINE1 RNA. The FTO protein or nucleic acid encoding for FTO may be provided at an amount effective to decrease an amount of histone modifications in the cell.

In certain aspects the amount of nucleic acid encoding FTO and/or FTO protein provided to the cell is approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 (or more or any range derivable therein) ng/μL of vehicle or ng/ml of vehicle. The vehicle may be any carrier suitable for contacting the protein and/or nucleic acid, such as water (including nuclease-free water), a buffered solution, a saline solution, DMSO, or other suitable liquid. The vehicle may be a cell culture medium. The nucleic acid and/or protein may be provided in a carrier that is capable of introducing the nucleic acid and/or protein into the cell, such as a liposome, or virus.

Also disclosed are methods for increasing methylation of LINE1 RNA in a cell. The method can comprises providing to the cell an effective amount of an FTO inhibitor. Also disclosed are methods for modifying expression level of a gene in a cell, where the method comprises providing to the cell an effective amount of an FTO inhibitor. Also disclosed are methods for decreasing chromatin accessibility in a cell, where the method comprises providing to the cell an effective amount of an FTO inhibitor. Also disclosed are methods for modifying development of a germ cell, where the method comprises providing to the cell an effective amount of an FTO inhibitor. The FTO inhibitor can be any one of FB23, FB23-2, CS1, CS2, and/or Dac51, or a combination thereof. The inhibitor can be provided at an amount effective to decrease chromatin accessibility in the cell. It is specifically contemplated that any of these may be excluded in an embodiment. In certain aspects the FTO inhibitor is provided at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 (or more or any range derivable therein) ng/μL of vehicle or ng/ml of vehicle or mg/mL of vehicle. The vehicle may be any carrier suitable for contacting the protein and/or nucleic acid, such as water (including nuclease-free water), a buffered solution, a saline solution, DMSO, or other suitable liquid. The vehicle may be a cell culture medium. The inhibitor can be provided at an amount effective to increase an amount of histone modifications in the cell. The inhibitor can be provided at an amount effective to modify the expression of a LINE1 element and/or a 2C gene. The cell can be any cell, such as an oocyte or fertilized cell. The cell can be from any source, such as a human, mouse, or livestock animal.

Methods of increasing zygote implantation in an animal are disclosed herein. Also disclosed are methods of increasing litter size, methods of improving an in vitro fertilization process, methods of increasing litter size and number in an animal, and/or methods of increasing zygote implantation rate. The methods can comprise implanting into an animal one or more fertilized cells that have been contacted, prior to the implanting of the fertilized cells, with exogenous fat mass and obesity-associated protein (FTO) and/or a nucleic acid molecule encoding FTO capable of transiently overexpressing exogenous FTO. The fertilized cell can be at any stage of development when the FTO protein and/or nucleic acid encoding FTO is introduced into the cells. The fertilized cell can be in a zygote, blastocyst, morula, or embryo stage of development when the FTO protein and/or nucleic acid encoding FTO is introduced. It is also disclosed that the FTO protein and/or nucleic acid prior can be introduced into the cells prior to the cells being fertilized, including into germ cells or gametes, such as an egg or a sperm.

The fertilized cells can be implanted when the fertilized cells are in a stage suitable for implantation, including in a blastocyst stage of development.

The amount of FTO protein and/or nucleic acid encoding FTO used to contact the cell may be an amount determined by one skilled in the art sufficient to effect the disclosed outcomes. The amount of FTO protein and/or nucleic acid encoding FTO used to contact the cell may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 (or more or any range derivable therein) ng/μL of vehicle or ng/ml of vehicle. The vehicle may be any carrier suitable for contacting the protein and/or nucleic acid, such as water (including nuclease-free water), a buffered solution, a saline solution, DMSO, or other suitable liquid. The vehicle may be a cell culture medium. The cell may be provided or contacted with an effective amount of the solution. The effective amount of the solution may be determined by one skilled in the art and can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 pL, nL, or μL, or any range derivable therein.

Also disclosed are aspects where FTO is replaced or used in addition with a different RNA demethylase. In some aspects, the different RNA demethylase is ALKBH5. The different RNA demthylase may be used in the same manner, including administered in the same manner, as FTO in certain aspects. Also disclosed are nucleic acid molecules encoding the different RNA demethylase.

The nucleic acid and/or protein may be provided in a carrier that is capable of introducing the nucleic acid and/or protein into the cell, such as a liposome, or virus. The nucleic acid and/or protein may be introduced into the cell by injecting the nucleic acid and/or protein into the cell, including by microinjection, which may be less than 1 μL. The nucleic acid may be introduced via viral vector and/or by transfection. The introducing can happen by contacting the cell with the protein and/or nucleic acid.

The amount of exogenous FTO protein, present in the fertilized cell from contacting the cell with the FTO protein and/or nucleic acid, may decrease in each cell as the fertilized cells expand and divide. In some aspects, the exogenous FTO is undetectable in the fertilized cells when the fertilized cells are in an embryonic stage of development.

The fertilized cells can be fertilized by any method, including by in vitro fertilization. The fertilized cells can also be produced from natural mating, artificial insemination, multiple ovulation and embryo transfer, somatic cell nuclear transfer, or other processes for fertilizing cells. The fertilized cells can be collected through non-surgical or surgical recovery techniques.

The animal may be any animal, including a human, non-human primate, livestock animal, companion animal, rodent, or endangered animal. The animal may be transgenic. The livestock animal may be a hooved animal, such as a cow, pig, sheep, bison, or deer. The livestock animal may be a bird, including any poultry, such as a chicken, duck, goose, pigeon, or turkey. The livestock animal may be an aquatic animal, such as a fish or a shellfish. A livestock animal may be an animal that is raised for food. The companion animal may be any domesticated animal such as a dog, cat, horse, or rabbit. The rodent may be any rodent, including those used in biomedical research such as a mouse or rat. The endangered animal may be any threatened or endangered wildlife animal, such as a panda, bison, polar bear, tiger, or lion. The endangered animal may be classified as an endangered species. It is specifically contemplated that any specific animal identified herein may be excluded in one or more aspects.

In certain aspects, the fertilized cells are xenotransplanted into the animal. For example, the fertilized cells can be from one species but implanted into an animal of another species. The animal may be used for medical xenotransplantation products such as a pig. Such animals may be an animal for xenotransplantation products.

In certain aspects, the nucleic acid molecule encoding FTO is a DNA molecule, an RNA molecule, or any nucleic acid analog molecule. The nucleic acid molecule encoding FTO may be an mRNA molecule. The DNA molecule may be an expression construct, including any expression construct capable of transiently overexpressing the FTO protein.

In certain aspects, the nucleic acid is provided to the cell or contacted with the cell with or without other solutes, carriers, activators, or other components, that one skilled in the art would employ to carry out the methods described herein. In certain aspects, the cells, including the fertilized cells, are implanted with or without other solutes, carriers, activators, or other components, that one skilled in the art would employ to carry out the methods described herein.

Also disclosed are the cells comprising the exogenous FTO protein and/or nucleic acid encoding the exogenous FTO protein. The cells may be generated using any method, including any method described herein to introduce the FTO protein and/or nucleic acid into the cell. The cells may be fertilized cells, germ cells, or gametes.

Also disclosed are compositions comprising any of the cells disclosed herein. The compositions may also comprise one or more reagents used for in vitro fertilization.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.” Any term used in singular form also comprise plural form and vice versa.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an aspect or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that aspects and aspects described in the context of the term “comprising” may also be implemented in the context of the term “consisting of” or “consisting essentially of.”

It is contemplated that any aspect discussed in this specification can be implemented with respect to any method or composition of the invention, and vice versa. Furthermore, compositions of the invention can be used to achieve methods of the invention.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other aspects and aspects are discussed throughout this application. Any aspect or aspect discussed with respect to one aspect of the disclosure applies to other aspects of the disclosure as well and vice versa.

It is specifically contemplated that any limitation discussed with respect to one aspect or aspect of the invention may apply to any other aspect or aspect of the invention. Furthermore, any composition of the invention may be used in any method of the invention, and any method of the invention may be used to produce or to utilize any composition of the invention. Aspects of an aspect set forth in the Examples are also aspects that may be implemented in the context of aspects discussed elsewhere in a different Example or elsewhere in the application, such as in the Summary of Invention, Detailed Description of the Aspects, Claims, and description of Figure Legends.

It is also specifically contemplated that certain limitations not specifically disclosed as being absent from the expressly defined systems, compositions and/or methods, but understood by a person of ordinary skill in the art as being necessarily absent, can also be excluded when practicing the aspects disclosed herein. For example, limitations not specifically disclosed as being absent but are required to be absent to achieve the aspects making the disclosed systems, compositions, and/or methods inventive over current art may be excluded. Such limitations include aspects that are currently standard aspects when performing similar methods and/or making or using similar systems or compositions.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific aspects presented herein.

FIGS. 1A-1E. m⁶A on LINE1 RNA is a major substrate of FTO in mESCs. (1A) Violin plots showing m⁶A level fold changes of hypermethylated m⁶A peaks on carRNAs upon Fto KO. P-values were determined using Wilcoxon's rank-sum tests. (1B) Boxplots showing expression fold changes of hypermethylated carRNAs versus other m⁶A-marked carRNAs upon Fto KO. P-values were determined using Wilcoxon signed-rank tests. (1C) Summary of repeat RNAs on chromatin upon Fto KO. Top: number of hypermethylated peaks, average m⁶A level, and expression. Middle: m⁶A level and expression fold changes (p-values were determined using Wilcoxon signed-rank tests). Bottom: expression fold changes of hypermethylated versus non-hypermethylated repeat RNAs (p-values were determined using Wilcoxon's rank-sum tests). (1D) Scatter plot showing the negative correlation of fold changes between m⁶A level and expression of LINE1 RNA upon Fto KO. LINE1 RNAs were categorized into 100 bins based on their ranked m⁶A level fold changes upon Fto KO. r refers to Pearson's correlation coefficient. P-value was calculated based on t-distribution. (1E) GSEA showing upregulated 2C genes (top) and downregulated ESC-high genes (bottom) from mRNA-seq upon Fto KO. NES, normalized enrichment score.

FIGS. 2A-2F. FTO regulates LINE1 RNA level through m⁶A demethylation. (2A) m⁶A peak profiles on m⁶A-marked LINE1 RNA in WT and Fto^{-/-} mESCs. (2B) Volcano plot showing differentially expressed subfamilies of m⁶A-marked repeat RNAs upon Fto KO. (2C) Cumulative distribution and boxplots (inset) showing nuclear LINE1 RNA lifetime in WT and Fto^{-/-} mESCs. (2D) Relative enrichment of LINE1 RNA by YTHDC1 from CLIP-qPCR in WT and Fto^{-/-} mESCs. P-values were determined using unpaired two-tailed t-tests; error bars and means±SD are shown for n=3 experiments. (2E) Cumulative distribution and boxplots (inset) showing LINE1 RNA transcription rate in WT and Fto^{-/-} mESCs. (2F) Cumulative distribution and boxplots (inset) showing the difference in transcription rate changes between m⁶A-marked and unmarked LINE1 RNA upon Fto KO. P-value was determined using Wilcoxon's rank-sum test. For (C) and (E), p-values were determined using Wilcoxon signed-rank tests.

FIGS. 3A-3D. FTO affects chromatin state through LINE1 RNA m⁶A demethylation. (3A) Left: DNase I-TUNEL assay showing more closed chromatin upon Fto KO. Scale bars, 20 μm. The nucleus was counterstained by DAPI. Representative images were selected from three independent experiments. Right: relative TUNEL intensity in WT and Fto^{-/-} mESCs. P-value was determined using an unpaired two-tailed t-test. TUNEL intensity was quantified by ImageJ. (3B) TUNEL signals of WT (grey) and Fto^{-/-} mESCs rescued by dCas13b-wtFTO with gRNA targeting LINE1 RNA (red) or control gRNA (blue). (3C) Profiles of H3K4Me3 and H3K9Me3 levels on LINE1 RNA loci from 3.0 kb upstream of the transcription start site (TSS) to 3.0 kb downstream of the transcription end site (TES) in WT and Fto^{-/-} mESCs. (3D) A schematic model showing how Fto KO affects LINE1 RNA abundance and local chromatin state.

FIGS. 4A-4D. Fto KO deactivates LINE1-containing genes by repressing intragenic LINE1 RNA. (4A) Boxplots showing gene expression fold changes upon Fto KO from caRNA-seq. Genes were categorized according to their genomic distance to the nearest LINE1 RNA with at least 10 reads. (4B) Scatter plot showing the negative correlation of fold changes between expression of LINE1-containing genes and m⁶A level of corresponding intragenic LINE1 RNA upon Fto KO. Intragenic LINE1 RNAs were categorized into 100 bins based on their ranked expression fold changes upon Fto KO. r refers to Pearson's correlation coefficient. P-value was calculated based on t-distribution. (4C) Boxplots showing fold changes of gene expression from caRNA-seq (left) and gene transcription rate (right) upon Fto KO. Genes were categorized into three groups: genes containing downregulated LINE1 RNA, genes near (<1 Mb) downregulated LINE1 RNA, and genes containing other LINE1 RNA. (4D) Profiles of H3K4Me3, H3K9Me3, H3K27Ac, and Pol II levels on loci of genes that contain downregulated LINE RNA from 3.0 kb upstream of the TSS and 3.0 kb downstream of the TES in WT and Fto^{-/-} mESCs. For (A) and (C), p-values were determined using Wilcoxon's rank-sum tests.

FIGS. 5A-5F. The FTO-LINE1 RNA axis plays critical roles during early development. (5A) Number of GV oocytes from 4-week-old WT and Fto^{-/-} mice. Error bars and means±SEM are shown for n=4 (WT) and n=6 (Fto−/− mice). (5B) Ratio of SN/NSN oocytes from 4-week-old WT (n=5) and Fto^{-/-} mice (n=4). (5C) Relative LINE1 RNA expression measured by RT-qPCR in WT and Fto^{-/-} oocytes. (5D) Left: DNase I-TUNEL assay showing more closed chromatin in oocytes upon Fto KO. Scale bars, 50 μm. The nucleus was counterstained by DAPI. Representative images were selected from three independent experiments. Right: relative TUNEL intensity in WT and Fto^{-/-} oocytes (n=12 each). pSN: partly surrounded nucleolus; NSN: non-surrounded nucleolus. (5E) Implantation rate (left) and E7.5 embryo rate (right) of Fto^{P+/M+}, Fto^{P+/M-}, Fto^{P-/M+}, and Fto^{P-/M-} zygotes. (5F) Relative LINE1 RNA expression measured by RT-qPCR in Fto^{P+/M+} and Fto^{P-/M-} morulae. For (A), (C), (D), and (F), p-values were determined using unpaired two-tailed t-tests; error bars and means±SD are shown for n=3 experiments in (C) and (F). For (B) and (E), p-values were determined using two-tailed z-tests.

FIGS. 6A-6N. FTO affects caRNA m⁶A methylation in mESCs. (6A) Representative images of WT, Fto^{-/-}, and Fto^{-/-} mice. (6B) Schematic diagram and an electrophoresis gel image of PCR products showing the validation of WT and Fto^{-/-} mESCs. (6C) Top: immunoblot assays showing the validation of Fto KO in mESCs. Bottom: FTO protein level in WT and Fto^{-/-} mESCs relative to GAPDH, quantified by ImageJ. (6D) Immunoblot assays showing the cell fractionation efficiency of WT and Fto^{-/-} mESCs. (6E-6G) m⁶A/A percentages quantified by UHPLC-MS/MS from WT and Fto^{-/-} mESCs for whole-cell polyadenylated RNA (E), chromatin-associated RNA after rRNA depletion (F), and non-ribosomal RNA from soluble nuclear extract (neRNA) (G). The m⁶A level of polyadenylated RNA only exhibited a minor increase upon Fto KO. (6H) Fluorescence images of the nucleus (counterstained with DAPI) and FTO protein (green) showing the predominant nuclear localization of the FTO protein in mESCs. Scale bars, 20 μm. Representative images were selected from three independent experiments. (6I-6L) Quality analyses of caRNA MeRIP-seq in WT and Fto^{-/-} mESCs. (I) Top consensus motifs identified from m⁶A peaks. (J) The overlaps of m⁶A peaks acquired from n=3 biological replicates showed high reproducibility. (K) The correlations of m⁶A levels among n=3 biological replicates showed high reproducibility. r refers to Pearson's correlation coefficient. (L) Distribution of m⁶A peaks in distinct genomic regions including promoter, exonic, intronic, transcription termination site (TTS), and intergenic regions annotated by HOMER. (6M) Volcano plot showing the differentially methylated caRNA m⁶A peaks upon Fto KO in mESCs. Hypermethylated peaks: 12,480; hypomethylated peaks: 8,848. (6N) Relative overall caRNA m⁶A level from caRNA MeRIP-seq in WT and Fto^{-/-} mESCs. caRNA. For (C), (E) to (G), and (N), p-values were determined using unpaired two-tailed t-tests; error bars and means±SD are shown for n=3 biological replicates in (C) and (N), for n=4 biological replicates in (E) to (G).

FIGS. 7A-7F. FTO regulates m⁶A level and expression of carRNAs in mESCs. (7A) Number of Fto KO-induced differential m⁶A peaks on carRNAs in WT and Fto^{-/-} mESCs. paRNA (+) and paRNA (−) denote sense paRNA and antisense paRNA, respectively. (7B) Boxplots showing m⁶A levels of carRNAs in WT and Fto^{-/-} mESCs. P-values were determined using Wilcoxon signed-rank tests. (7C) Fraction of m⁶A-marked carRNAs with differential expression in WT and Fto^{-/-} mESCs. (7D) Scatter plots showing the negative correlations between fold changes in transcript abundance and m⁶A levels of carRNAs upon Fto KO in mESCs. carRNAs within each group were categorized into 100 bins based on their ranked fold changes of m⁶A levels upon Fto KO. (7E) Scatter plots showing the negative correlations of fold changes between m⁶A level and expression of carRNAs with both hypermethylated and downregulated expression upon Fto KO in mESCs. These plots revealed the direct effect on carRNAs caused by Fto KO. (7F) A schematic model showing the elevated m⁶A level and repressed transcript level of chromatin-associated repeat RNAs (the most responsive group to Fto KO among carRNAs) upon Fto depletion. For (D) and (E), r refers to Pearson's correlation coefficient. P-values were calculated based on t-distribution.

FIGS. 8A-8I. FTO binds to LINE1 RNA and regulates its m⁶A level and expression in mESCs. (8A) The transcript abundance of overall LINE1 RNA on chromatin quantified in CPM values from caRNA-seq in WT and Fto^{-/-} mESCs. (8B) Relative m⁶A fold enrichment of LINE1 RNA on chromatin measured by MeRIP-qPCR (left), and relative expression of LINE1 RNA on chromatin measured by RT-qPCR (right), both in WT and Fto^{-/-} mESCs. (8C) Relative expression of LINE1 RNA and Gapdh (as control) measured by RT-qPCR in different subcellular fractions. (8D) Fluorescence images, intensity profile, and 2D intensity histogram of LINE1 RNA (magenta) and FTO protein (green) showing their colocalization. Scale bars, 10 μm. r refers to Pearson's correlation coefficient determined by Coloc2 in ImageJ. P-value was determined using a one-sample t-test. (8E) CLIP-qPCR showing LINE1 RNA bound to FTO (using an antibody against endogenous FTO) and IgG (as negative control) in WT mESCs. (8F) Left: FISH showing reduced LINE1 RNA level upon Fto KO. Scale bars, 20 μm. Right: boxplots showing the relative FISH intensity quantified by ImageJ in WT and Fto^{-/-} mESCs. (8G) Relative m⁶A fold enrichment of total LINE1 RNA measured by MeRIP-qPCR (left), and relative expression of total LINE1 RNA measured by RT-qPCR (right), both in WT and Fto^{-/-} mESCs. (8H) Top: immunoblotting assays showing L1ORF1p in WT and Fto^{-/-} mESCs. Bottom: L1ORF1p level relative to Tubulin quantified by ImageJ. (8I) WT mESCs were treated with DMSO (as control) or an FTO inhibitor (FB23-2) for 72 hours. Left: relative m⁶A fold enrichment of total LINE1 RNA measured by MeRIP-qPCR. Right: relative expression of total LINE1 RNA measured by RT-qPCR. For (D) and (F), the nucleus was counterstained with DAPI; representative images were selected from three independent experiments. For (A) to (C) and (E) to (I), p-values were determined using unpaired two-tailed t-tests; error bars and means±SD are shown for n=3 biological replicates in (A), (C), (E), and (H), and for n=4 biological replicates in (B), (G), and (I).

FIGS. 9A-9I. LINE1 RNA m⁶A is a physiological substrate of FTO across mammalian tissues. (9A) Negative correlations between the m⁶A level and LINE1 RNA expression in mouse (left) and human (right) tissues, respectively. (9B) The negative correlation between the m⁶A level of repeat RNAs and Fto expression in mouse tissues. (9C) LINE1 RNA showed both high m⁶A level and expression among various repeat RNAs. (9D) The negative correlation between m⁶A fold enrichment and relative LINE1 RNA expression in mouse tissues. (9E) The negative correlation between LINE1 RNA m⁶A fold enrichment and relative Fto expression (left), and the positive correlation between the relative expression of LINE1 RNA and Fto (right) in mouse tissues. (9F) The negative correlation between the LINE1 RNA m⁶A level and FTO expression (left), and the positive correlation between the expression of LINE1 RNA and FTO (right) in human tissues. (9G) No clear correlation exists between the m⁶A level of repeat RNAs and Alkbh5 expression in mouse tissues. (9H) No clear correlation exists between LINE1 RNA m⁶A fold enrichment and relative Alkbh5 expression (left) or between the relative expression of LINE1 RNA and Alkbh5 (right) in mouse tissues. (9I) No clear correlation exists between LINE1 RNA m⁶A level and ALKBH5 expression (left) or between the expression of LINE1 RNA and ALKBH5 (right) in human tissues. Relatedly, ALKBH5 was shown to mediate mRNA m⁶A demethylation in these tissues (13). In Hela cells, ALKBH5 can remove 5′ UTR m⁶A on LINE1 RNA to suppress LINE1 retrotransposition, while FTO was shown not to affect LINE1 mobility (67). For (A) to (I), the correlations are presented in the scatter plots; r refers to Pearson's correlation coefficient; p-values were calculated based on t-distribution. For (A), (B), (C), (F), (G), and (I), m⁶A level and expression were calculated from RNA-seq and m⁶A MeRIP-seq datasets, respectively, from mouse (CRA001315) or human (CRA001962) tissues (13). For (D), (E), and (H), m⁶A fold enrichment and relative expression were measured by MeRIP-qPCR and RT-qPCR, respectively.

FIGS. 10A-10N. Fto KO largely recapitulates the effects of LINE1 ASO treatment in mESCs. (10A) GSEA showing global upregulation upon Fto KO of LINE1 RNA-targeted genes revealed by ChIRP-seq (top) and LINE1 sequence-enriched genes (bottom). NES, normalized enrichment score. These findings resemble reported observations upon LINE1 ASO treatment (27, 28). Together with FIG. 1E, these transcriptomic data indicate FTO functions through similar pathways compared to LINE1 RNA in mESCs. (10B) MA plot showing transcriptomic changes analyzed from mRNA-seq upon Fto KO in mESCs. 2C genes were upregulated and several pluripotency genes were downregulated. (10C and 10D) RNA-seq datasets of LINE1 ASO treatment were analyzed from GSE100939 (27). (C) Scatter plot showing mRNA expression fold changes upon Fto KO or LINE1 ASO in mESCs. Fto KO and LINE1 ASO showed consistent effects on the transcriptome. Fto KO led to lower activation of 2C genes but greater downregulation of pluripotency genes compared to LINE1 ASO treatment. (D) Heatmap showing the consistent changes of most key ESC genes between Fto KO and LINE1 ASO in mESCs. (10E) Expression changes of the downregulated (top) or upregulated (bottom) genes caused by Fto KO across indicated embryonic developmental stages. Downregulated genes upon Fto KO were highly expressed at the ESC stage while upregulated genes were highly expressed in early 2C or 2C stages. P-values were determined using Wilcoxon signed-rank tests, **** p<0.0001 was labeled by comparing indicated stage to each of the other stages. RNA-seq datasets of early embryonic development were analyzed from GSE66390 (68). (10F) Cell-cycle analysis of WT and Fto^{-/-} mESCs. Consistent with LINE1 ASO (27), Fto KO led to a reduced proportion of cells in S phase. P-value was determined using a Chi-square test. (10G) AP staining of WT and Fto^{-/-} mESCs cultured for three days after initial plating. Scale bar: 200 μm. Fto KO led to only slightly reduced AP positive mESCs. (10H) The colony morphology of WT and Fto^{-/-} mESCs cultured for three days after passaging with the same cell number. Scale bar: 200 μm. Fto^{-/-} mESCs grew slower than WT mESCs after passaging. (10I) Representative images (left) and boxplots (right) showing the increased diameters of EBs upon Fto KO measured by ImageJ four days after EB induction in mESCs. Scale bar: 200 μm. (10J) Relative expression of total LINE1 RNA quantified by RT-qPCR in WT mESCs with reverse complement ASO (RC ASO, as control) or LINE1 ASO treatment. Error bars and means±SD are shown for n=3 biological replicates. (10K) Proliferation of Fto^{-/-} mESCs (blue solid line) and WT mESCs with the treatment of control ASO (grey solid line) or LINE1 ASO (grey dotted line). (10L) Differentiation of Fto^{-/-} mESCs (blue solid line) and WT mESCs with the treatment of control ASO (grey solid line) or LINE1 ASO (grey dotted line). Mouse embryoid body (mEB) formation is represented by proliferation upon differentiation induction. (10M) Left: Bright field and dark field epi-fluorescent images of chimeras generated with WT and Fto^{-/-} mESCs. Scale bar: 1000 μm. Right: percentage of red fluorescent protein (RFP) positive cells. Error bars and means±SD are shown for n=3 (WT) and n=6 (Fto^{-/-}) mice. (10N) The ratio of chimeric fetus generated with WT and Fto^{-/-} mESCs. Fto^{-/-} mESCs exhibited a weak ability to generate chimeric mice, indicating a reduced in vivo differentiation potency. For (I), (J), (M), and (N), p-values were determined using unpaired two-tailed t-tests. Representative images were selected from three independent experiments for (G) to (I). Notably, Fto KO leads to lower 2C gene activation but more downregulated ESC-high genes compared to LINE1 ASO treatment; LINE1 ASO also severely impairs colony formation (27), while Fto KO showed minor effects. The inventors also suspect FTO and ASO have different target specificities; Fto KO and ASO treatment may cause downregulation of different LINE1 RNA to very different extents.

FIGS. 11A-11N. FTO-mediated m⁶A demethylation regulates abundance of LINE1 RNA subfamilies on chromatin. (11A) The distribution of m⁶A peaks on LINE1 RNA with different evolutionary ages. m⁶A exhibited an enriched distribution on young LINE1 RNA (<3.5 Myr). (11B) Boxplots showing the m⁶A level of old and young LINE1 in WT mESCs (left) and m⁶A level fold changes upon Fto KO (right). Young LINE1 showed higher m⁶A levels in WT mESCs and greater changes in m⁶A levels upon Fto KO. (11C) The distribution of m⁶A peaks on the LINE1 RNA with different lengths. m⁶A exhibited an enriched distribution on full-length LINE1 (˜7000 bp). (11D) Scatter plot showing the negative correlation between the average m⁶A level of LINE1 subfamilies and their mean divergences in WT mESCs. Each dot represents the average m⁶A level and mean divergence within one LINE1 subfamily. r refers to Pearson's correlation coefficient. P-value was calculated based on t-distribution. (11E) Metagene profiles of the differential m⁶A peak along overall LINE1 RNA on chromatin upon Fto KO in mESCs. Hypermethylated peaks were enriched around the 5′-end while hypomethylated peaks were slightly enriched around both the 5′-end and middle region. (11F) Metagene profiles of hypomethylated m⁶A peaks along overall LINE1 RNA on chromatin upon Mettl3 KO in mESCs. Differential peaks caused by Fto KO showed similar distributions to hypomethylated peaks upon Mettl3 KO, supporting the model that FTO mainly reverses m⁶A deposited by METTL3 mainly to the 5′ region of LINE1. caRNA MeRIP-seq data was analyzed from GSE133600 (22). (11G) LINE1 RNA of the indicated young subfamilies on chromatin were analyzed. Left three columns: heatmaps showing the average m⁶A level and expression in WT mESCs, and m⁶A fold changes upon Fto KO, respectively. Right: heatmap showing the expression changes between WT and Fto^{-/-} mESCs. The histogram in the heatmap indicates the z-score scaling across the rows. Z-score was calculated as a measure of distance in standard deviations from the mean. Notably, the expression difference is larger than the m⁶A level difference for certain subfamilies, indicating a trans-regulation mechanism. (11H) Heatmaps showing consistent RNA expression downregulation for indicated LINE1 subfamilies when separating multiple-mapped reads (top) and unique-mapped reads (bottom) from caRNA-seq, suggesting the stability of the bioinformatic analysis on these subfamilies. Z-scores were presented as in (G). Significantly downregulated young LINE1 subfamilies upon Fto KO in FIG. 2B are shown. (11I) Metagene profile of the differential m⁶A peak along selected FTO-targeted LINE1 subfamilies upon Fto KO in mESCs. (11J) Left: relative RNA m⁶A fold enrichment of top FTO-targeted LINE1 subfamilies on chromatin measured by MeRIP-qPCR in WT and Fto^{-/-} mESCs. Right: relative RNA expression of selected FTO-targeted LINE1 subfamilies on chromatin measured by RT-qPCR in WT and Fto^{-/-} mESCs. (11K-11N) dCas13b-FTO (wild-type or inactive mutant) constructs with LINE1 gRNA or control gRNA were applied in Fto^{-/-} mESCs to modulate the m⁶A methylation level of LINE1 RNA. LINE1 gRNA was designed to target the young LINE1 RNA subfamilies most responsive to Fto KO. (K) A schematic model showing the dCas13b-FTO site-specific demethylation system. (L-N) Left: relative m⁶A fold enrichment of overall LINE1 RNA on chromatin (L), L1Md_Tf on chromatin (M), and L1Md_A on chromatin (N), measured by MeRIP-qPCR in WT and Fto^{-/-} mESCs. Right: relative expression of overall LINE1 RNA on chromatin (L), L1Md_Tf on chromatin (M), and L1Md_A (N) on chromatin measured by RT-qPCR in WT and Fto^{-/-} mESCs. Only targeting dCas13b-wfFTO to LINE1 RNA in Fto^{-/-} mESCs could rescue the m⁶A level and expression of overall LINE1 RNA and selected FTO-targeted LINE1 subfamilies on chromatin. For (J) and (L) to (N), p-values were determined using unpaired two-tailed t-tests; error bars and means±SD are shown for n=4 biological replicates in (J), and for n=3 biological replicates in (L) to (N).

FIGS. 12A-12K. FTO-mediated m⁶A demethylation regulates lifetime and transcription of FTO-targeted LINE1 subfamilies. (12A) Applying dCas13b-wtFTO with gRNA targeting LINE1 RNA (LINE1 gRNA) in Fto^{-/-} mESCs led to increased lifetime of overall LINE1 RNA compared to control gRNA. 112 was calculated using the one phase decay model in GraphPad Prism. (12B) Applying dCas13b-wtFTO with LINE1 gRNA in Fto^{-/-} mESCs led to reduced YTHDC1 binding compared to control gRNA. (12C) Venn diagram showing the overlap between LINE1 RNA with reduced transcription rate (pink) and decreased lifetime (blue). Almost half of the LINE1 with decreased lifetime also showed reduced transcription rate, indicating transcription is associated with RNA decay for a notable portion of LINE1 RNA. (12D) Percentage of young LINE1 subfamilies in LINE1 RNA with reduced transcription rate. Almost half of the LINE1 RNAs that show reduced transcription rate are young LINE1. (12E) Cumulative distribution and boxplots (inset) showing the RNA transcription rate of selected FTO-targeted LINE1 subfamilies in WT and Fto^{-/-} mESCs from nascent RNA-seq. P-values were determined using Wilcoxon signed-rank tests. LINE1 subfamilies showed reduced transcription rates upon Fto KO. (12F) Cumulative distribution and boxplots (inset) showing the transcription rate differences between m⁶A-marked and unmarked ERVK (left) or Alu (right) upon Fto KO in mESCs from nascent RNA-seq. Only minimal differences can be observed between m⁶A-marked and unmarked transcripts upon Fto KO, suggesting these repeat RNAs may be indirectly affected by FTO or LINE1 RNA through trans-regulation. P-values were determined using Wilcoxon's rank-sum tests. (12G) ChIRP-qPCR showing relative DNA binding by LINE1 RNA in WT and Fto^{-/-} mESCs. Fto KO led to reduced DNA binding of overall LINE1 RNA and selected FTO-targeted LINE1 subfamilies. Malat1 was used as the negative control. (12H) DRIP-qPCR showing the relative levels of LINE1 RNA: DNA hybrids in WT and Fto^{-/-} mESCs. Fto KO led to reduced R-loop formation of overall LINE1 RNA and selected FTO-targeted LINE1 subfamilies. Reduced DNA association and R-loop formation were consistent with the decreased transcription rate of LINE1 RNA. (12I-12K) For overall LINE1 RNA and selected FTO-targeted LINE1 subfamilies, applying dCas13b-wtFTO with LINE1 gRNA in Fto^{-/-} mESCs led to increased transcription rate (I), increased DNA binding (J), and recovered R-loop formation (K), compared to control gRNA. For (B), (G), (H), (J), and (K), p-values were determined using unpaired two-tailed t-tests; error bars and means±SD are shown for n=3 biological replicates.

FIGS. 13A-13J. FTO regulates global transcription rate and chromatin accessibility in mESCs. (13A) Left: 5-Ethynyl Uridine (EU) labeling followed by fluorescence imaging showing reduced global nascent RNA synthesis upon Fto KO. Scale bars, 20 μm. The nucleus was counterstained by DAPI. Right: boxplots showing relative EU intensity quantified by ImageJ in WT and Fto^{-/-} mESCs. (13B) Relative 4-thiouridine (4sU)/A ratio in total RNA quantified by UHPLC-MS/MS in WT and Fto^{-/-} mESCs. (13C) Left: EU labeling followed by fluorescence imaging showing reduced global nascent RNA synthesis upon FTO inhibitor treatment. Right: boxplots showing relative EU intensity. (13D) Left: DNase I-TUNEL assay followed by fluorescence imaging showing more closed chromatin upon FTO inhibitor treatment. Right: boxplots showing relative TUNEL intensity. For (C) and (D), WT mESCs were treated with FTO inhibitor (FB23-2) or DMSO (as control) for 72 hours. Scale bars, 20 μm. The nucleus was counterstained by DAPI. EU and TUNEL intensity were quantified by ImageJ. (13E) DNase I-TUNEL assay followed by flow cytometry showing more closed chromatin upon LINE1 RNA knockdown with LINE1 ASO (purple) compared to RC ASO (grey) in WT mESCs. LINE1 ASO caused less chromatin closure compared to Fto KO. (13F) TUNEL signal of Fto^{-/-} mESCs after applying dCas13b-mutFTO with LINE1 gRNA (red) or control gRNA (blue) were compared to WT mESCs (grey). No obvious change in TUNEL signal was observed. (13G-13I) WT mESCs were treated with an FTO inhibitor (FB23-2) for 48 hours. (G) Relative m⁶A fold enrichment of LINE1 RNA measured by MeRIP-qPCR. (H) Relative expression of LINE1 RNA measured by RT-qPCR. (I) Left: DNase I-TUNEL assay followed by fluorescence imaging showing gradual chromatin closure. Scale bars, 20 μm. Right: boxplots showing relative TUNEL intensity quantified by ImageJ. (13J) A schematic model showing a plausible mechanism by which FTO inhibition leads to repressed LINE RNA and closed chromatin. For (A), (C), (D), and (I), representative images were selected from three independent experiments. For (A), (C), (D), and (G) to (I), p-values were determined using unpaired two-tailed t-tests; for (G) and (H), error bars and means±SD are shown for n=3 biological replicates.

FIGS. 14A-14E. Overall changes in ATAC-seq and histone marks in WT and Fto^{-/-} mESCs. (14A) Density plots showing the overall ATAC-seq signal at peak center quantified with RPKM in WT and Fto^{-/-} mESCs. (14B) Circos plots showing ATAC peaks along the genome in WT and Fto^{-/-} mESCs. (14C) Volcano plot showing differential ATAC peaks upon Fto KO in mESCs. Significantly increased peaks are shown in red (p<0.01:889; FDR<0.05:164) and significantly decreased peaks are shown in blue (p<0.01:1397; FDR<0.05:251). Raw peaks were merged into united peak sets and more significantly decreased peaks were obtained. P-values of differential peaks were determined by FeatureCounts and DESeq2. (14D) Gene Ontology (GO) analysis of top enriched biological processes (blue) and molecular functions (red) of gained-closed regions. Gained-closed regions were defined by 1 kb sliding windows (57) from ATAC-seq in WT and Fto^{-/-} mESCs. 4,683 gained-closed regions were identified upon Fto KO. (14E) Left: immunoblotting assays showing histone modification levels in WT and Fto^{-/-} mESCs. Right: histone modification levels in WT and Fto^{-/-} mESCs relative to histone H3, quantified by ImageJ. Consistent with the finding of more closed chromatin, Fto KO led to slightly decreased active marks and increased repressive marks. P-values were determined using unpaired two-tailed t-tests; error bars and means±SD are shown for n=3 independent immunoblots.

FIGS. 15A-15C. Genomic features of gained-closed regions obtained from ATAC-seq upon Fto KO. (15A-15C) Cross-analysis of ATAC-seq and ChIP-seq datasets of histone marks and transcription factors. ChIP-seq binding sites sorted by gained-closed regions from ATAC-seq were plotted in heatmap views. (15A) Gained-closed regions upon Fto KO from ATAC-seq were enriched with H3K4Me1, H3K4Me3, H3K27Ac, Pol II, YY1, and P300, but not H3K9Me3, H3K27Me3, or H3K36Me3 regions identified from ChIP-seq in WT mESCs. YY1: Yin Yang 1, a transcription factor; EP300, E1A Binding Protein P300, a transcriptional co-activator. Previous studies have uncovered these two proteins can be recruited by caRNA to promote transcription (22, 29, 30). METTL3 binding sites were also enriched in gained-closed regions with Fto depletion, which may explain some of the hypomethylated m⁶A peaks upon Fto KO. (15B) Fto KO-induced gained-closed regions were also enriched with reduced levels of H3K4Me3, H3K27Ac, and decreased Pol II, YY1, and EP300 binding from ChIP-seq upon Fto KO. Consistently, regions with reduced METTL3 chromatin binding upon Fto depletion were enriched with Fto KO-induced gained-closed regions. Together, these findings are consistent with the observed role of FTO in transcriptional activation in mESCs. (15C) Fto KO-induced gained-closed regions were further enriched with elevated levels of H3K4Me3, H3K27Ac, and increased YY1 and EP300 binding upon Mettl3 KO, indicating these effects are m⁶A-dependent. For (A) and (C), datasets from ENCODE or GSE133600 (22) were used.

FIGS. 16A-16H. FTO-mediated m⁶A demethylation of LINE1 RNA regulates local chromatin state. (16A-16C) ChIP signal was profiled on LINE1 RNA loci from 3.0 kb upstream of the transcription start site (TSS) to 3.0 kb downstream of the transcription end site (TES). (A) Profiles of H3K27Ac, H3K4Me1, H4K20Me3, and RNA polymerase II (Pol II) levels at loci with m⁶A-marked LINE1 RNA in WT and Fto^{-/-} mESCs. H3K27Ac and Pol II levels were reduced upon Fto KO. (B-C) Profiles of H3K4Me3 (B) or H3K9Me3 (C) levels at loci with young and old LINE1 RNA in WT and Fto^{-/-} mESCs. H3K4Me3 level decreased mainly at loci with young LINE1 RNA while old LINE1 RNA loci exhibited a greater increase in H3K9Me3 level, suggesting young and old LINE1 loci are dominated by different histone marks. (16D) Boxplots showing fold changes of H3K4Me3, H3K27Ac, and H3K9Me3 levels at the TSS region of LINE1 subfamilies upon Fto KO in mESCs. Loci of selected FTO-targeted LINE1 subfamilies showed increased levels of H3K4Me3 and H2K27Ac, and decreased H3K9Me3 levels upon Fto KO. Loci of a non-m⁶A LINE1 subfamily LIM2b were used as negative control. LIM2b loci and loci of LAPez-int, an ERVK subfamily shown to be regulated by METTL3 (23-25), showed negligible or opposite changes in levels of H3K4Me3, H2K27Ac, and H3K9Me3 upon Fto KO. (16E) Applying dCas13b-wtFTO with LINE1 gRNA in Fto^{-/-} mESCs led to increased levels of H3K4Me3 and H3K27Ac, and decreased H3K9Me3 levels at overall LINE1 RNA loci and loci with selected FTO-targeted LINE1 subfamilies compared to control gRNA. (16F) Profiles of YY1 (top) and EP300 (bottom) chromatin binding at their peak center and flanking 0.5 kb regions in WT and Fto^{-/-} mESCs. Fto KO led to globally decreased YY1 and EP300 binding. (16G) Boxplots showing the decreased chromatin binding of YY1 and EP300 at m⁶A-marked LINE1 RNA loci upon Fto KO in mESCs, indicating FTO-mediated m⁶A demethylation of LINE1 RNA regulates local chromatin by affecting transcription factor binding. This supports the model that the transcription activation role of FTO-mediated LINE1 RNA m⁶A demethylation in mESCs involves the binding of EP300 and YY1 to facilitate the opening of local chromatin (22, 29, 30). (16H) CLIP-qPCR showing LINE1 RNA bound by YY1 (left) and EP300 (right) in WT and Fto^{-/-} mESCs. IgG was used as normalization control. Fto KO led to reduced YY1 and EP300 binding to overall LINE RNA and selected FTO-targeted LINE1 subfamilies. Consistent with previous reports (22, 29, 30), reduced LINE1 RNA binding by transcription factors may be responsible for their decreased local chromatin binding. For (D) and (G), p-values were determined using Wilcoxon signed-rank tests. For (E) and (H), p-values were determined using unpaired two-tailed t-tests; error bars and means±SD shown for n=3 biological replicates.

FIGS. 17A-17G. LINE1 RNA is a functionally relevant substrate of FTO in EB differentiation and self-renewal of mESCs. (17A) Relative LINE1 RNA expression measured by RT-qPCR (left), and relative Fto expression measured by RT-qPCR (right), measured at days 0 and 6 of EB differentiation in WT and Fto^{-/-} mESCs. Both LINE1 RNA and Fto showed increased expression after EB differentiation; LINE1 RNA showed greater decreases upon Fto KO in mESCs after six days of EB differentiation. P-values were determined using unpaired two-tailed t-tests; error bars and means±SD shown for n=4 biological replicates. (17B) Heatmap showing the expression of key differentiation markers measured by RT-qPCR in WT and Fto^{-/-} mESCs at day 0 and day 6 during EB differentiation. Decreased expression of pluripotency markers Oct4 and Sox2 were observed upon Fto KO, with most markers showing greater expression changes after six days of EB differentiation. (17C and 17D) Differentiation of WT mESCs (grey solid line), and Fto^{-/-} mESCs rescued by dCas13b-wtFTO (C) or dCas13b-mutFTO (D) with LINE1 gRNA (blue dotted line) or control gRNA (blue solid line). Only targeting dCas13b-wfFTO to LINE1 RNA in Fto^{-/-} mESCs could partially rescue the induced differentiation observed in Fto^{-/-} mESCs. mEB formation was represented by cell proliferation upon differentiation induction. (17E) Hierarchical Clustering of the expression changes of differentiation markers with LINE1 ASO treatment in WT mESCs or dCas13b rescue experiments in Fto^{-/-} mESCs. WT mESCs treated with RC ASO and Fto^{-/-} mESCs rescued by dCas13b-wtFTO with LINE1 gRNA were clustered together by Euclidean distance and average linkage. (17F and 17G) Proliferation of WT mESCs (grey solid line), and Fto^{-/-} mESCs rescued by dCas13b-wtFTO (F) or dCas13b-mutFTO (G) with LINE1 gRNA (blue dotted line) or control gRNA (blue solid line). Only targeting dCas13b-wtFTO to LINE1 RNA in Fto^{-/-} mESCs could partially rescue the reduced proliferation.

FIGS. 18A-18H. FTO regulates transcription and local chromatin state of LINE1-containing genes. (18A) Genes were categorized into two groups: genes that contained indicated LINE1 subfamilies and genes near (<1 Mb) indicated LINE1 subfamilies. Boxplots show gene expression fold changes upon Fto KO from caRNA-seq in mESCs. Upon Fto KO, LINE1-containing genes of selected FTO-targeted LINE1 subfamilies showed decreased expression compared to genes that do not contain these LINE1 subfamilies. (18B) Scatter plot showing the positive correlation between expression fold changes of LINE1-containing genes and the corresponding intragenic LINE1 RNA from caRNA-seq upon Fto KO in mESCs. Intragenic LINE1 RNA was categorized into 100 bins based on its ranked fold changes of transcript abundance upon Fto KO. r refers to Pearson's correlation coefficient. P-value was calculated based on t-distribution. (18C) Cumulative distribution and boxplots (inset) showing the transcription rate of LINE1-containing genes from nascent RNA-seq in WT and Fto^{-/-} mESCs. LINE1-containing genes showed significantly reduced transcription rates upon Fto KO. (18D) Boxplots showing gene transcription rate fold changes upon Fto KO from nascent RNA-seq in mESCs. Genes were grouped based on their distances to the nearest expressed LINE1 RNA with at least 10 reads. “0 kb” represents LINE1 RNA located within the gene bodies along the genome (genes are termed as LINE1-containing gene). Fto KO led to decreased transcription rates of LINE1-containing genes compared to genes in other groups. (18E) Genes were categorized as in (A). Boxplots show the gene transcription rate difference upon Fto KO from nascent RNA-seq in mESCs. Upon Fto KO, LINE1-containing genes of selected FTO-targeted LINE1 subfamilies showed significantly decreased transcription rates compared to genes that do not contain these LINE1 subfamilies. (18F) Boxplots showing level fold changes of H3K4Me3, H3K27Ac, and H3K9Me3 at the TSS region of intragenic and intergenic LINE1 upon Fto KO from ChIP-seq in mESCs. Intragenic LINE1 RNA loci showed more significantly decreased H3K27Ac levels and more significantly increased H3K9Me3 levels upon Fto KO, while intergenic LINE1 RNA loci showed more significantly decreased H3K4Me3 levels. P-values were determined using Wilcoxon signed-rank tests. (18G) Cumulative distributions and boxplots (inset) showing the lifetime fold changes (left) from nuclear RNA lifetime-seq and the transcription rate differences (right) from nascent RNA-seq between m⁶A-marked intragenic and intergenic LINE1 RNA changes upon Fto KO in mESCs, respectively. Intragenic LINE1 RNA showed a greater decrease in lifetime while both intragenic and intergenic LINE1 RNAs showed comparable transcription rate decreases upon Fto KO. (18H) ChIP signal of indicated histone marks was profiled at loci of genes that contain FTO-targeted LINE1 RNA subfamilies from 3.0 kb upstream of the TSS to 3.0 kb downstream of the TES. Fto KO led to decreased levels of H3K4Me3 and H3K27Ac, and increased H3K9Me3 levels at loci of genes that contain selected FTO-targeted LINE1 RNA subfamilies.

FIGS. 19A-19G. IGV profiles showing methylation, abundance, and local chromatin state of selected down-LINE1 loci and down-LINE1-containing genes. (19A) Venn diagram showing the overlap between genes that contain downregulated LINE1 RNA and downregulated genes upon Fto KO from caRNA-seq in mESCs. (19B) GO analysis of downregulated genes that contain downregulated LINE1 RNA (1,434 genes were identified). GO terms were enriched with transcription, cell differentiation, and neuronal/cardiac development, which indicates these downregulated genes that contain downregulated LINE1 RNA are responsible for ESC and developmental phenotypes caused by Fto KO. (19C-19F) IGV tracks showing ChIP-seq, caRNA-seq, and caRNA m⁶A-seq for (C) Esrrb, (D) Nek5, (E) Phf3, and (F) Zfp982, as selected downregulated genes that contain downregulated LINE1 RNA. RNA and m⁶A profiles were separated into forward (f) and reverse (r) strands according to the strand they were mapped to, respectively. Increased m⁶A level and decreased expression on chromatin were observed for intragenic LINE1 RNAs. Decreased H3K4me3, H3K27ac, and Pol II levels were observed at both intragenic LINE1 loci and TSS regions of LINE1-containing genes. All genes except for Phf3 also showed increased H3K9Me3 levels at LINE loci. Esrrb is known to play essential roles in the pluripotency network of mESCs (69). All three other genes have important functions in mESCs or during early development. (19G) Decreased transcription rates were observed for all four genes from nascent RNA-seq.

FIGS. 20A-20J. Applying dCas13b-wtFTO reverses lifetime changes of selected intragenic LINE1 RNA and transcription changes of LINE1-containing genes. (20A) CLIP-qPCR showing LIMD3 (in Essrb) bound by YTHDC1 in WT and Fto^{-/-} mESCs (left) and Fto^{-/-} mESCs rescued by dCas13b-wfFTO with LINE1 gRNA or control gRNA (right). IgG was used as a normalization control. Fto KO led to increased YTHDC1 binding to LIMD3 (in Essrb), which was reversed by targeting dCas13b-wtFTO to LINE1 RNA. (20B) Lifetime of LIMD3 (in Essrb) in WT mESCs (grey) and Fto^{-/-} mESCs rescued by dCas13b-wtFTO with LINE1 gRNA (red) or control gRNA (blue). Fto KO led to reduced lifetime of LIMD3 (in Essrb), which was reversed by targeting dCas13b-wtFTO to LINE1 RNA. (20C) Relative changes in the transcription rate, H3K4Me3, H3K9Me3, H3K27Ac, and Pol II levels of Esrrb after applying a dCas13b-wtFTO with LINE1 gRNA or control gRNA. Targeting dCas13b-wtFTO to LINE1 RNA led to increased gene transcription rate, elevated levels of local H3K4me3, H3K27ac, and pol II binding, together with reduced local H3K9Me3 levels. (20D) CLIP-qPCR showing L1MdTf_I (in Phf3) bound by YTHDC1 in WT and Fto^{-/-} mESCs (left) and Fto^{-/-} mESCs rescued by dCas13b-wfFTO with LINE1 gRNA or control gRNA (right). IgG was used as a normalization control. Fto KO led to increased YTHDC1 binding to L1MdTf_I, which can be reversed by targeting dCas13b-wfFTO to LINE1 RNA. (20E) Lifetime of L1MdTf_I (in Phf3) in Fto^{-/-} mESCs rescued by dCas13b-wtFTO with LINE1 gRNA (red) or control gRNA (blue) and WT mESCs (grey). Fto KO led to reduced lifetime of L1MdTf_I (in Phf3), which was reversed by targeting dCas13b-wfFTO to LINE1 RNA. (20F) Only minimal changes in lifetime were observed for Esrrb or Phf3 transcripts from nuclear RNA lifetime-seq. (20G) Reduced lifetime was observed for L1Md.A_III (in Nek5) and L1MdF_I (in Zfp982) from nuclear RNA lifetime-seq. The inventors could design primers for these two subfamilies of LINE1 RNA but could not design primers to specifically target L1Md.A_III locus contained by Nek5 or L1MdF_I locus contained by Zfp982 because of multiple targeting. (20H) Applying dCas13b-wtFTO with LINE1 gRNA in Fto^{-/-} mESCs led to increased transcription rates of Nek5, Phf3, and Zfp982 compared to control gRNA. (20I) Applying dCas13b-wfFTO with LINE1 gRNA in Fto^{-/-} mESCs led to increased local levels H3K4Me3 and H3K27Ac, and decreased local H3K9Me3 levels on Nek5, Phf3, and Zfp982 loci compared to control gRNA. (20J) Relative expression of Dub1, Zscan4, and MERVL RNA after applying dCas13b-wtFTO with LINE1 gRNA or control gRNA in Fto^{-/-} mESCs. All three targets were reported to be repressed by the scaffolding trans-regulation of LINE1 RNA (27). Targeting dCas13b-wtFTO to LINE1 RNA in Fto^{-/-} mESCs reduced their expression. Note that 2C genes Dub1 and Zscan4 do not contain LINE1 RNA. MERVL is a 2C-specific transposon that is also repressed by LINE1 RNA. This result supports the notion that FTO-mediated m⁶A demethylation regulates the scaffolding trans-regulation of LINE1 RNA. For (A), (C), (D), (I), and (J), p-values were determined using unpaired two-tailed t-tests; error bars and means±SD are shown for n=3 biological replicates. For (B) and (E), t_{1/2} was calculated using the one phase decay model in GraphPad Prism.

FIGS. 21A-21L. FTO regulates LINE1 m⁶A and RNA level, as well as chromatin state in brain tissues. (21A) Left: relative LINE1 RNA m⁶A fold enrichment in total RNA isolated from the cerebellum of WT and Fto^{-/-} mice. Right: relative LINE1 RNA expression in the same samples. (21B and 21C) Left: DNase I-TUNEL assay followed by fluorescence imaging showing more closed chromatin under 10× (B) or 40× (C) scope in cerebellum slice from WT and Fto^{-/-} mice. Scale bars, 120 μm for (B) and 30 μm for (C). Right: bar plots (B) or boxplots (C) showing relative TUNEL intensity. Relative TUNEL intensities were quantified as an average of all the cells in one image for (B), or signals in each cell nucleus for (C) by ImageJ. (21D) Left: relative LINE1 RNA m⁶A fold enrichment in total RNA isolated from the hippocampus of WT and Fto^{-/-} mice. Right: relative expression of LINE1 RNA in the same samples using RT-qPCR. (21E and 21F) DNase I-TUNEL assay followed by fluorescence imaging showing more closed chromatin under 10× (E) or 40× (F) scope in hippocampus slices from WT and Fto^{-/-} mice. Scale bars, 120 μm for (E) and 30 μm for (F). Right: bar plots (E) or boxplots (F) showing relative TUNEL intensity. Relative TUNEL intensities were quantified as an average of all the cells in one image (E), or signals in each cell nucleus for (F) by ImageJ. (21G) Left: relative LINE1 RNA m⁶A fold enrichment in total RNA isolated from WT and Fto^{-/-} aNSCs. Right: the relative LINE1 RNA expression in the same samples. (21H) Left: DNase I-TUNEL assay followed by fluorescence imaging showing more closed chromatin measured upon Fto KO in aNSCs. Right: boxplots showing the relative TUNEL intensity. (21I) Left: EU labeling followed by fluorescence imaging showing reduced global nascent RNA synthesis upon Fto KO in aNSCs. Right: boxplots showing the relative EU intensity. For (H) and (I), measurements are from WT and Fto^{-/-} aNSCs. Scale bars, 20 μm. Relative EU or TUNEL intensity to DAPI were quantified in each nucleus by ImageJ. (21J) Top: immunoblot assays showing the H3K4Me3 level of the cerebellums and hippocampi from WT and Fto^{-/-} mice; bottom: H3K4Me3 protein levels relative to histone H3, quantified by ImageJ. (21K) Relative LINE1 RNA expression in total RNA from WT and Fto^{-/-} aNSCs before (Proli) and after (Diff) differentiation, respectively. Consistent with observations in EB differentiation of mESCs, LINE1 RNA showed increased expression after differentiation and more significantly decrease upon Fto KO in aNSCs after differentiation. (21L) Top: immunoblot assay showing FTO level in WT aNSCs before (Proli) and after (Diff) differentiation. Bottom: FTO protein levels relative to GAPDH quantified by ImageJ. P-values were determined using unpaired two-tailed t-tests; error bars and means±SD are shown for n=4 biological replicates in (A), (D), (G), and (K), for n=3 biological replicates in (B), (E), (J), and (L). For (A), (D), and (J), m⁶A fold enrichment and relative expression were measured by MeRIP-qPCR and RT-qPCR, respectively. For (B), (C), (E), (F), (H), and (I), the nucleus was counterstained by DAPI; representative images were selected from three independent experiments.

FIGS. 22A-22K. The FTO-LINE1 RNA axis plays important roles in oocyte development. (22A) Percentages of embryo genotypes from heterozygote intercrosses. The Fto^{-/-} embryo ratio is significantly lower than the theoretical value. P-value was determined using a Chi-squared test for 221 embryos collected in n=5 independent experiments. (22B) The pups per plug (left) and the surviving offspring after 24 hours (right) from crosses of WT males with WT and Fto^{-/-} females, respectively. Maternal Fto KO showed a greater fatality with reduced pup numbers after mating with WT male mice, with all surviving Fto^{-/-} pups dying shortly after birth. Error bars and means±SEM are shown for n=6 pairings of mice. (22C) Representative images of the ovaries from 4-week-old WT and Fto^{-/-} mice (left); HE staining of the ovaries from 4-week-old (middle) or 7-week-old (right) WT and Fto^{-/-} mice. Ovaries from Fto^{-/-} mice displayed defects with a slightly smaller size and reduced number of primordial follicles. (22D) Fto (line) and LINE1 RNA (bar) expression quantified in CPM values was analyzed throughout oocyte development from total RNA-seq datasets (GSE75738) (70). (22E) Boxplots showing significantly reduced LINE1 RNA levels (RPKM) from RNA-seq in GV and MII oocytes from Fto^{-/-} mice compared to WT control, respectively. P-values were determined using Wilcoxon signed-rank tests. (22F) Scatter plot showing the expression fold changes of LINE1 subfamilies in GV or MII oocytes upon Fto KO. Significantly downregulated LINE1 subfamilies upon Fto KO in mESCs were labeled in blue. RNA expression changes of LINE1 subfamilies in oocytes from Fto^{-/-} mice compared to WT control showed a consistent pattern with those due to Fto KO in mESCs. (22G) Numbers of MII oocytes collected from 4-week-old Fto^{-/-} mice and WT controls. MII numbers showed a large variation in Fto^{-/-} mice after superovulation. Error bars and means±SEM are shown for n=7 Fto^{-/-} mice and n=6 WT controls. (22H) Left: fluorescence imaging showing meiosis defects of MII oocytes from Fto^{-/-} mice compared to WT control. Scale bars, 50 μm. Right: increased percentages of chromosome misalignment and spindle collapse of MII oocytes from Fto^{-/-} mice compared to WT controls. Error bars and means±SD are shown for three independent experiments. (22I) Scatter plots showing the positive correlation between transcript abundance fold changes of LINE1-containing genes and the corresponding intragenic LINE1 RNA in GV (left) and MII (right) oocytes from Fto^{-/-} mice compared to WT controls. Intragenic LINE1 RNA was categorized into 100 bins based on its ranked fold changes in transcript abundance upon Fto KO. r refers to Pearson's correlation coefficient. P-values were calculated based on t-distribution. (22J) Boxplots showing the gene expression fold changes from RNA-seq in GV (left) and MII (right) oocytes of Fto^{-/-} mice compared to WT controls. Genes were categorized into three groups: genes that contain downregulated LINE1 RNA (denoted down-LINE1-containing genes, “Down” and “Containing”), genes near (<1 Mb) downregulated LINE1 RNA (“Down”), and genes that contain LINE1 RNA not downregulated (“Containing”). Fto KO led to significantly decreased expression of genes that contain downregulated LINE1 RNA compared to other genes. P-values were determined using Wilcoxon's rank-sum tests. (22K) GO analysis of downregulated genes that contain downregulated LINE1 RNA in GV or MII oocytes from Fto^{-/-} mice compared to WT controls. For (B), (G), and (H), p-values were determined using unpaired two-tailed t-tests.

FIGS. 23A-23R. FTO-mediated LINE1 RNA m⁶A demethylation plays important roles during embryonic development. (23A) A schematic model showing the intercross workflow. (23B) In vitro fertilization (IVF) rate (in percent) from intercrosses between WT MII oocytes and Fto^{-/-} sperm. Fto^{-/-} sperm shows decreased activity but most fertilized zygotes can still develop to the blastocyst stage. Because the loss of Fto in sperm led to decreased in vitro fertilization rates, the inventors employed the intracytoplasmic sperm injection (ICSI) technique to fertilize oocytes. (23C) Percentage of blastocyst progression (E3.5, left; E4.5, right) of Fto^{P+/M+}, Fto^{P+/M-}, Fto^{P-/M+}, and Fto^{P-/M-} zygotes. Paternal, maternal, or double depletion of Fto all led to slightly hindered blastocyst progression. (23D) Representative images showing implantation defects upon loss of Fto. Either paternal or maternal loss of Fto led to reduced implantation rate. Scale bar: 1000 μm for decidua, 500 μm for embryos. Representative images were selected from two independent experiments. No E7.5 embryo was obtained from embryos with double depletion of both Fto alleles. (23E) Boxplots showing reduced LINE1 RNA level (RPKM) from RNA-seq in Fto^{P+/M+} morulae compared to Fto^{P-/M-} morulae. (23F) Scatter plot showing the RNA expression fold changes of LINE1 subfamilies in mESCs or morulae upon Fto KO. Significantly downregulated LINE1 subfamilies upon Fto KO in mESCs are labeled in blue. RNA expression changes of LINE1 subfamilies between Fto^{P-/M-} morulae and Fto^{P+/M+} morulae are consistent with those between Fto^{-/-} mESCs and WT mESCs. (23G) RT-qPCR showing elevated Zscan4 and MERVL RNA levels from Fto^{P+/M+} and Fto^{P-/M-} morulae. Error bars and means±SD are shown for n=3 experiments. (23H) Boxplots showing increased MERVL RNA level (RPKM) in Fto^{P-/M-} morulae compared to Fto^{P+/M+} morulae. P-value was determined using a Wilcoxon signed-rank test. (23I) Boxplot showing the gene expression fold changes from RNA-morulae seq in Fto^{P-/M-} compared to Fto^{P+/M+} morulae. Genes were categorized into three groups: genes that contain downregulated LINE1 RNA (denoted down-LINE1-containing genes, “Down” and “Containing”), genes near (<1 Mb) downregulated LINE1 RNA (“Down”), and genes that contain LINE1 RNA not downregulated (“Containing”). Fto KO led to significantly decreased expression of genes that contain downregulated LINE1 RNA compared to other genes. P-values were determined using Wilcoxon's rank-sum tests. (23J) Scatter plot showing the positive correlation between expression fold changes of LINE1-containing genes and corresponding intragenic LINE1 RNA in Fto^{P-/M-} morulae compared to Fto^{P+/M+} morulae. Intragenic LINE1 RNA was categorized into 100 bins based on its ranked fold changes of transcript abundance upon Fto KO. r refers to Pearson's correlation coefficient. P-values were calculated based on t-distribution. (23K) Expression of selected downregulated genes that contain downregulated LINE1 RNA from RNA-seq in Fto^{P+/M+} and Fto^{-/-} morulae. Lin28b, Tet2, and Gsk3b were identified as downregulated genes that contain downregulated LINE1 RNA upon Fto KO in both mESCs and morulae, supporting the model that Fto depletion causes delayed 2C-exit and developmental defects. P-values were determined using DESeq2. (23L) GO analysis of downregulated genes that contain downregulated LINE1 RNA in Fto^{P-/M-} morulae compared to Fto^{P+/M+} morulae. (23M) A schematic model (created with BioRender) showing the dcas13b-wfFTO rescue experiments during early development: dcas13b-wtFTO with control gRNA or LINE1 gRNA was microinjected into Fto^{-/-} MII oocytes. After the ICSI with Fto^{-/-} sperm and in vitro development, Fto^{P-/M-} morulae (labeled as LINE1 gRNA, red, or control gRNA, blue; based on the microinjection procedure at MII stage) were collected for subsequent experiments. (23N) Representative images showing Fto^{P+/M+}, Fto^{P-/M-}, and Fto^{P-/M-} embryos treated with dCas13b-wtFTO and LINE1 gRNA or control gRNA at different stages. Representative images were selected from two independent experiments. (23O-23R) Fto^{P-M-} morulae were derived from Fto^{-/-} MII oocytes injected with dCas13b-wtFTO and control gRNA (blue) or LINE1 gRNA (red). (O) Relative expression of LINE1 in the total RNA isolated from morulae measured by RT-qPCR. Targeting dCas13b-wtFTO to LINE1 RNA in Fto^{-/-} MII oocyte can elevate the expression of LINE1 and MERVL RNA in Fto^{P-/M-} embryos after developing to morula stage. (P) Left: DNase I-TUNEL assay followed by fluorescence imaging showing more open chromatin in an Fto^{P-/M-} morula with LINE1 gRNA compared to one with control gRNA. Scale bars, 20 μm. The nucleus was counterstained by DAPI. Representative images were selected from n=8 morulae (control gRNA) and n=10 morulae (LINE1 gRNA). Right: boxplots showing the relative TUNEL intensity in morulae. Relative TUNEL intensity was quantified by ImageJ. Targeting LINE1 RNA with dCas13b-wtFTO in Fto^{-/-} MII led to more open chromatin in Fto^{P-/M-} morulae, which is associated with increased LINE1 RNA expression. (Q) RT-qPCR showing reduced Zscan4 and MERVL RNA levels in the total RNA isolated from Fto^{P-/M-} morulae with LINE1 gRNA compared to those with control gRNA. Targeting dCas13b-wtFTO to LINE1 RNA in Fto^{-/-} MII oocytes reduced the expression of Zscan4 and MERVL RNA after developing to the morula stage. (R) Relative expression of selected downregulated genes that contain downregulated LINE1 RNA in the total RNA isolated from morulae measured by RT-qPCR. Means for n=2 biological replicates. These three targets were downregulated genes that contain downregulated LINE1 RNA shared between Fto^{P-/M-} morulae compared to Fto^{P+/M+} morulae and Fto^{-/-} mESCs compared to WT mESCs. Targeting dCas13b-wtFTO to LINE1 RNA in Fto^{-/-} MII oocytes elevated the expression of Lin28b, Tet2, and Gsk3b in Fto^{P-/M-} morulae. For (E) and (H), p-values were determined using Wilcoxon signed-rank tests. For (G) and (O) to (R), p-values were determined using unpaired two-tailed t-tests; means are shown for n=2 experiments in (O), (Q), and (R).

FIGS. 24A-24C. Fto KO leads to accelerated decay and repressed transcription of carRNAs. (24A) Cumulative distribution and boxplots (inset) showing carRNA lifetime changes in WT and Fto^{-/-} mESCs. carRNAs within each group showed reduced lifetimes upon Fto KO. (24B) Cumulative distribution and boxplots (inset) showing carRNA transcription rate in WT and Fto^{-/-} mESCs. carRNAs within each group showed reduced transcription rate upon Fto KO. (24C) Cumulative distribution and box plots (inset) showing the difference in transcription rate between m⁶A-marked and unmarked carRNAs. P-values were determined using Wilcoxon's rank-sum tests. Only m⁶A-marked repeat RNAs showed a distinct left-shift (indicating a significantly decreased transcription rate compared to unmarked repeat RNAs) in the cumulative curve upon Fto KO. For (A) and (B), p-values were determined using Wilcoxon signed-rank tests.

FIGS. 25A-25C. FTO-mediated changes in m⁶A level and expression of carRNAs associated with METTL3 and YTHDC1. (25A) Negative correlations are observed in m⁶A level fold changes of carRNAs between hypermethylation upon Fto KO and hypomethylation upon Mettl3 KO, indicating m⁶A peaks directly demethylated by FTO were likely deposited by METTL3. (25B) The positive correlations in m⁶A level fold changes of carRNAs between hypomethylation upon Fto KO and upon Mettl3 KO, indicating hypomethylated m⁶A peaks upon Fto KO were likely caused by the hindered accessibility of METTL3 to its targets. For (A) and (B), carRNAs within each group and LINE1 RNA were categorized into 50 bins based on their ranked fold changes of m⁶A levels upon Fto KO. Correlations for Repeat RNA and LINE1 RNA are also shown in scatter plots. (25C) Negative correlations are observed in transcript abundance fold changes between carRNAs downregulated upon Fto KO and carRNAs upregulated upon Ythdc1 cKO. carRNAs within each group and LINE1 RNA were categorized into 50 bins based on their ranked fold changes in transcript abundance upon Fto KO. For (A) to (C), r refers to Pearson's correlation coefficient. P-values were calculated based on t-distribution.

FIGS. 26A-26L. Polyadenylated mRNA and snRNA are unlikely to be primary targets of FTO in mESCs. (26A) Boxplots showing ca-mRNA m⁶A level in WT and Fto^{-/-} mESCs. (26B) Boxplots showing m⁶A levels of exons and introns in ca-mRNA in WT and Fto^{-/-} mESCs. (26C) Volcano plot showing the differentially methylated m⁶A peaks on ca-mRNA upon Fto KO in mESCs. (26D) Boxplots showing the expression fold changes of hypermethylated and other m⁶A-marked ca-mRNA upon Fto KO in mESCs. P-value was determined using a Wilcoxon's rank-sum test. For (A) to (D), m⁶A and expression levels of chromatin-associated mRNA (ca-mRNA), most of which are pre-mRNA, were evaluated. (26E and 26F) Quality analyses of mRNA m⁶A MeRIP-seq data in WT and Fto^{-/-} mESCs. (E) Top consensus motifs identified from m⁶A peaks. (F) The overlaps of m⁶A peaks acquired from n=2 replicates showed high reproducibility. (26G) Volcano plot showing the differential m⁶A methylation upon Fto KO in mESCs. (26H) mEB formation represented by cell proliferation upon differentiation induction for WT and Mettl4 mESCs. (26I) Proliferation of WT and Mettl4 mESCs. (26J) HE staining of the ovaries from 3-week-old WT and Mettl4 “mice. Representative images were selected from three independent experiments. No obvious defects were observed upon Mettl4 KO. (26K) Comparable numbers of GV oocytes were collected from 9-week-old Mettl4” mice and WT controls. P-value was determined using an unpaired two-tailed t-test; error bars and means±SEMs are shown for n=6 mice. (26L) Comparable numbers of SN and NSN GV oocytes were collected from 9-week-old Mettl4^{-/-} mice. Error bars and means±SEMs are shown for n=3 mice. The SN/NSN ratio is similar to normal WT mice at this age. For (A) and (B), p-values were determined using Wilcoxon signed-rank tests.

FIGS. 27A-27P. FTO knockdown leads to more open chromatin and increased transcription in Mel624 cells through demethylation of mRNAs encoding histone modifiers. (27A-27C) The m⁶A MeRIP-seq datasets from human cell lines that include HEK293T cells, GOS cells, HT29 cells, Jurkat cells, K562 cells, U2OS cells, U251 cells, and WPMY cells were analyzed from CRA001315 (13). Most of these cell lines are human cancer cell lines. (A) Scatter plot showing the negative correlation between LINE1 RNA m⁶A level and FTO expression. (B) Scatter plot showing no clear correlation between expression of LINE1 RNA and FTO. (C) Scatter plot showing no clear correlation between m⁶A level and expression of LINE1 RNA. (27D) The m⁶A/A percentage measured by UHPLC-MS/MS of caRNA after depletion of rRNA (left) and polyadenylated RNA (right) in control and Mel624 cells with FTO knockdown. (27E) Left: DNase I-TUNEL assay followed by fluorescence imaging showing more open chromatin in Mel624 cells with FTO knockdown compared to control. Right: boxplots showing relative TUNEL intensity. (27F) Left: EU labeling followed by fluorescence imaging showing elevated global nascent RNA synthesis in Mel624 cells with FTO knockdown compared to control. Right: boxplots showing relative EU intensity. For (E) and (F): Scale bars, 30 μm. The nucleus was counterstained by DAPI. Representative images were selected from three independent experiments. EU and TUNEL intensity were quantified in each nucleus by ImageJ. (27G) Relative LINE1 RNA expression quantified by RT-qPCR in control and Mel624 cells with FTO knockdown. (27H) Relative expression of total LINE1 RNA quantified by RT-qPCR in in control and Mel624 cells with LINE1 ASO knockdown. (27I) LINE1 RNA knockdown with LINE1 ASO (red) in Mel624 cells led to minimal changes in chromatin openness measured by DNase I-TUNEL assay followed by flow cytometry compared to control ASO (grey). (27J) Density plots showing the overall ATAC-seq signal at peak center quantified by RPKM in control and Mel624 cells with FTO knockdown. (27K) Volcano plot showing the differential ATAC peaks in Mel624 cells with FTO knockdown compared to control. Significantly increased peaks are shown in red (p<0.01:8097; FDR<0.05:261) and significantly decreased peaks are shown in blue (p<0.01:278; FDR<0.05:5). p and FDR values of differential peaks were determined using FeatureCounts and DESeq2. Raw peaks were merged into united peak sets and more significantly increased peaks than decreased peaks were obtained. (27L) Gained-open regions upon FTO knockdown from ATAC-seq were enriched with H3K27Ac regions identified from ChIP-seq. ChIP-seq binding sites sorted by gained-open regions from ATAC-seq were plotted in heatmap views. A higher H3K27Ac level was observed for these regions when comparing FTO knockdown to control. Gained-open regions were defined by 1 kb sliding windows along the genome. (27M) Immunoblot assays showing dysregulated protein levels of H3K27Ac and H3K27Me3 in Mel624 cells with FTO knockdown compared to control. (27N) Heatmap showing dysregulated expression of EP300, KDM6B, and EZH2 from mRNA-seq in Mel624 cells with FTO knockdown compared to control. (27O) IGV tracks showing increased levels of m⁶A peaks on EP300, KDM6B, and EZH2 transcripts in Mel624 cells with FTO knockdown compared to control. (27P) Knockdown of YTHDF2 in shFTO Mel624 cells can rescue the decreased lifetime of EZH2, while knockdown of IGF2BP1-3 in shFTO Mel624 cells can rescue the increased lifetime of EP300 and KDM6B. For (D) to (H), p-values were determined using unpaired two-tailed t-tests. For (D), (G), and (H), error bars and means±SD are shown for n=4 experiments.

FIGS. 28A-28H. FTO regulates LINE1 RNA m⁶A, LINE1 RNA abundance, and LINE1 RNA association to DNA. (28A) Left: FISH showing reduced LINE1 RNA level upon Fto KO. Scale bars, 20 μm. Right: boxplots showing the relative FISH intensity quantified by ImageJ in WT and Fto^{-/-} mESCs. (28B) Relative m⁶A fold enrichment of total LINE1 RNA measured by MeRIP-qPCR (left), and relative expression of total LINE1 RNA measured by RT-qPCR (right), both in WT and Fto^{-/-} mESCs. (28C) Top: immunoblotting assays showing L1ORF1p in WT and Fto^{-/-} mESCs. Bottom: L1ORF1p level relative to Tubulin quantified by ImageJ. (28D) WT mESCs were treated with DMSO (as control) or an FTO inhibitor (FB23-2) for 72 hours. Left: relative m⁶A fold enrichment of total LINE1 RNA measured by MeRIP-qPCR. Right: relative expression of total LINE1 RNA measured by RT-qPCR. (28E) ChIRP-qPCR showing relative DNA binding by LINE1 RNA in WT and Fto^{-/-} mESCs. Fto KO led to reduced DNA binding of overall LINE1 RNA and selected FTO-targeted LINE1 subfamilies. Malat1 was used as the negative control. (28F) DRIP-qPCR showing the relative levels of LINE1 RNA: DNA hybrids in WT and Fto^{-/-} mESCs. Fto KO led to reduced R-loop formation of overall LINE1 RNA and selected FTO-targeted LINE1 subfamilies. Reduced DNA association and R-loop formation were consistent with the decreased transcription rate of LINE1 RNA. (28G and 28H) For overall LINE1 RNA and selected FTO-targeted LINE1 subfamilies, applying dCas13b-wtFTO with LINE1 gRNA in Fto^{-/-} mESCs led to increased DNA binding (G) and recovered R-loop formation (H), compared to control gRNA.

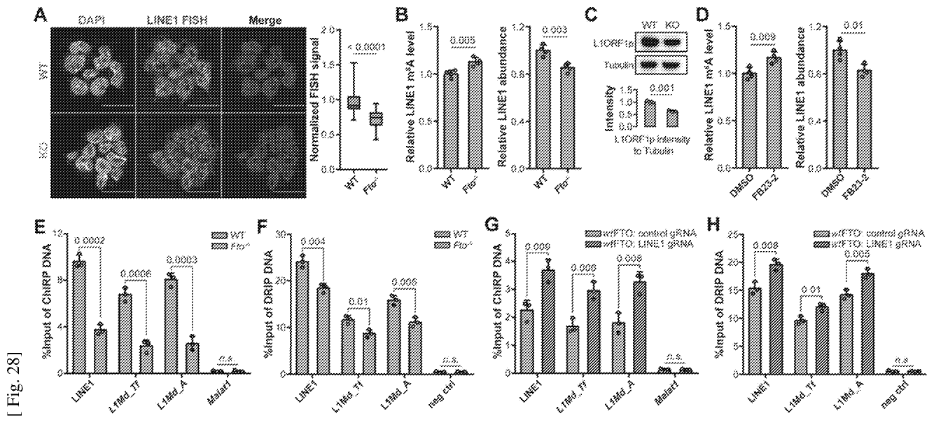

FIGS. 29A-29C. FTO transient OE in zygotes increases the body weights of female but not male F1 pups. (29A) Schematic illustration of the experimental procedure. (B and C) Body weights of female (29B) and male (29C) F1 pups born from Fto mRNA injected or uninjected zygotes.

FIGS. 30A-30B. Overexpression of FTO-GFP fusion protein in early embryos. (30A) Schematic illustration of the experimental procedure. (30B) Detection of GFP signal under fluorescence microscope in embryos with or without successful injection or translation of Fto-Gfp fusion mRNA. The GFP positive embryos can be selected for downstream analysis. Scale bar: 20 μm.

FIG. 31. FTO OE increases MERVL expression and promotes its entry into the nucleus.

FIGS. 32A-32H. FTO transient OE in zygotes increased the implantation ratio at E7.5 and increased the body weights of embryos at E12.5. (32A) The development of FTO-Ctrl, FTO-KO and FTO-OE embryos at E4.5. (32B) The average numbers of ICM and TE cells in FTO-Ctrl, FTO-KO and FTO-OE embryos at E4.5. (32C) Images of deciduas and embryos of FTO-Ctrl and FTO-OE embryos at E7.5, related with the left panel of D. (32D) The ratio of embryo/decidua was calculated at E7.5 BDF1 cross BDF1 for left panel and C57 cross DBA2 for right panel. (32E) Weights of FTO-Ctrl and FTO-OE embryos and placentas at E12.5. (32F) Placental development of the FTO-Ctrl and FTO-OE groups at E12.5. The arrowhead points to the labyrinth layer. (32G) Body weight tracing of FTO-Ctrl and FTO-OE pups after birth to mature adult. (32H) Glucose Tolerance Test for normal diet and high-fat diet mice (male only) at week 8-9. The high-fat diet were served for 2 weeks before the test (n=6-10). (32I) Representative photographs of FTO-Ctrl and FTO-OE mice at week 18 after high-fat diet for 12 weeks. (32J) Body weight of FTO-Ctrl and FTO-OE mice at week 18 after high-fat diet for 12 weeks.

### DETAILED DESCRIPTION OF THE INVENTION

Disclosed herein are functionally relevant substrates and mechanisms of RNA m⁶A demethylation through FTO useful in mammalian development. The disclosure supports LINE1 RNA as a major substrate of FTO in mESCs. Aspects show FTO additionally mediates m⁶A demethylation of other carRNAs to affect gene expression. In contrast to certain cancer cells where FTO can be hijacked to mediate mRNA m⁶A demethylation (7-12), which may dominate chromatin state regulation (34) (supplementary text and FIG. S22), aspects herein show that FTO-mediated m⁶A demethylation maintains LINE1 RNA abundance in mESCs. This can contribute to promoting local chromatin openness and activating LINE1-containing genes. Aspects herein show that the FTO-LINE1 RNA axis is functionally relevant in mouse oocyte and embryonic development. In addition to m⁶A, RNA 5-methylcytosine oxidation is also known to affect transcription of ERVL and ERVL-associated genes in mESCs (35), suggesting a potential widespread presence of regulation through retrotransposon RNA modifications (36).

Also disclosed is FTO overexpression (transient or stable) applied to in vitro fertilization (IVF), artificial insemination, cloning, or to improving the natural fertilities and birth outcomes of mammals. Also included are IVF, artificial insemination, or improving the natural fertilities of humans. Aspects directed to artificial insemination can also apply to sex-sorted semen (also known as sexed semen), a commonly used method in livestock reproduction.

In addition to humans, the non-human mammals in which FTO overexpression methods can be used include but are not limited to livestock (domesticated agricultural animals), companion animals, endangered wildlife animals, and animals raised for research products or biomedical products.

### A. Proteins

Disclosed herein are FTO proteins, and nucleic acids encoding such proteins. The FTO proteins may be exogenous proteins that are introduced to a cell to effect RNA methylation patterns and development of the cell, as described herein.

As used herein, a “protein” or “polypeptide” refers to a molecule comprising at least five amino acid residues. In some aspects, wild-type versions of a protein or polypeptide are employed. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically disclosed that a modified/variant protein or polypeptide may be altered with respect to one activity or function yet retain a wild-type activity or function in other respects, such as enzymatic activity.

The protein, including an FTO protein, may be isolated directly from the organism of which it is native, produced by recombinant DNA/exogenous expression methods, or produced by solid-phase peptide synthesis (SPPS) or other in vitro methods. In particular aspects, there are isolated nucleic acid segments and recombinant vectors incorporating nucleic acid sequences that encode a polypeptide (e.g., an FTO polypeptide or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of an FTO protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is disclosed that polypeptides, including the FTO polypeptide used herein, may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of any of the gene products identified in Table 1.

In some aspects, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of any of the gene products identified in Table 1.

In some aspects, the protein, polypeptide, or nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of any of the gene products identified in Table 1.

In some aspects, the polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of any of the gene products identified in Table 1 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of any of the gene products identified in Table 1.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of the gene products identified in Table 1 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of the gene products identified in Table 1.

In some aspects there is a polypeptide, or nucleic acid encoding the polypeptide, from the gene products identified at Table 1 with a substitution, deletion, or insertion at one or more positions including at positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, (or any range derivable therein) so long as the polypeptide has FTO activity corresponding to at least or at most 10, 20, 30, 40, 50, 60, 70, 80, 90, 100, 110, 120, 130 percent of an unsubstituted protein or wild-type FTO protein.

Disclosed herein are uses of an FTO protein. The FTO protein used may be any FTO protein homolog capable of eliciting the desired outcomes, including eliciting the improvements in in vitro fertilization (such as increasing implantation rates). The FTO protein used may be from any organism. In certain aspects, the FTO protein sequence is from an animal. In certain aspects, the FTO protein sequence is from a mammal. In certain aspects, the FTO protein sequence is from a rodent. In certain aspects, the FTO protein sequence is from a livestock animal. In certain aspects, the FTO protein sequence is from a companion animal. In certain aspects, the FTO sequence is from a wildlife animal. In certain aspects, the FTO protein sequence is from a primate. In certain aspects, the FTO protein sequence is from a human. In certain aspects, the FTO protein sequence is from a starfish. In certain aspects, the FTO protein sequence is from a species listed in Table 1.

TABLE 1
Identifiers of FTO Sequences, all of which
are incorporated herein by reference
Accession Number (species)

NM_001080432.3 (Homo sapiens - Human)
XM_015126148.2 (Macaca mulatta - Rhesus monkey)
NM_011936.2 (Mus musculus - Mouse)
NM_001039713.1 (Rattus norvegicus - Rat)
NM_001098142.1 (Bos taurus - Cattle)
XM_019979409.1 (Bos taurus - Cattle)
XM_005890937.1 (Bos mutus - Cattle)
NM_001112692.1 (Sus scrofa - Pig)
XM_038659338.1 (Canis Lupis familiaris - Dog)
NM_001104931.1 (Ovis aries - sheep)
XP_022097537.1 (Acanthaster planci - Starfish)
XP_038078604 (Patiria miniate - Starfish)
XP_033625687.1 (Asterias rubens - Starfish)
ALR88588.1 (Saccoglossus kowalevskii - Acorn worm)
XP_001420808.1 (Ostreococcus lucimarinus)
XM_009303351.3 (Danio rerio - zebrafish)
XP_022840077.1 (Ostreococcus tauri)
OUS43030.1 (Ostreococcus tauri)
CAD8812083.1 (Ostreococcus mediterraneus)
XP_002502764.1 (Micromonas commode)
XP_003060875.1 (Micromonas pusilla)
CAD8435953.1 (Micromonas pusilla)
CAE3374532.1 (Micromonas Polaris)

The FTO protein and/or nucleic acid encoding the FTO protein introduced or provided to the cells of the disclosure may be the corresponding sequence to the species of cell. For example, the FTO protein comprising the human sequence of FTO may be introduced into a human cell. Similarly, a nucleic acid encoding the human sequence of FTO may be introduced into human cell.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is disclosed that in compositions of the disclosure, there is between about 0.001 mg and about 10 mg of total polypeptide, peptide, and/or protein per mL. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 ng/ml or mg/mL or more (or any range derivable therein).

### 1. Variant Polypeptides

The following is a discussion of changing the amino acid subunits of a protein, including an FTO protein, to create an equivalent, or even improved, second-generation variant polypeptide or peptide. For example, certain amino acids may be substituted for other amino acids in a protein or polypeptide sequence with or without appreciable loss of enzymatic activity capacity. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus disclosed by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### 2. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further aspects, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within +2 is included. In some aspects of the present disclosure, those that are within #1 are included, and in other aspects of the present disclosure, those within +0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain aspects, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0=1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain aspects, the substitution of amino acids whose hydrophilicity values are within ±2 are included, in other aspects, those which are within #1 are included, and in still other aspects, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an enzyme with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some aspects of the disclosure, amino acid substitutions are made that: (1) reduce susceptibility to proteolysis, (2) reduce susceptibility to oxidation, (3) alter binding affinity for forming protein complexes or for enzymatic activity, (4) alter ligand binding affinities, and/or (5) confer or modify other physicochemical or functional properties on such polypeptides. For example, single or multiple amino acid substitutions (in certain aspects, conservative amino acid substitutions) may be made in the naturally occurring sequence. Substitutions can be made in that portion of the protein that lies outside the domain(s) forming intermolecular contacts. In such aspects, conservative amino acid substitutions can be used that do not substantially change the structural characteristics of the protein or polypeptide (e.g., one or more replacement amino acids that do not disrupt the secondary structure that characterizes the native antibody).

### B. Nucleic Acids

Certain aspects concern nucleic acids that encode an FTO protein. The nucleic acid can be delivered to a cell in a manner suitable for the cell to express an amount of the FTO protein sufficient to exert an effect on the cell. The nucleic acid can be a DNA, RNA, or any nucleic acid analog capable of expressing a protein. In some aspects, the nucleic acid comprises at least one sequence region, such as a promoter, allowing expression in a specific cell type, such as a fertilized cell.

In certain aspects, nucleic acid sequences can exist in a variety of instances such as: isolated segments and recombinant vectors of incorporated sequences or recombinant polynucleotides encoding an enzyme (such as FTO), or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain aspects, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### 1. Mutation

Changes can be introduced by mutation into a nucleic acid, thereby leading to changes in the amino acid sequence of a polypeptide (e.g., an enzyme or enzyme derivative) that it encodes. Mutations can be introduced using any technique known in the art. In one aspect, one or more particular amino acid residues are changed using, for example, a site-directed mutagenesis protocol. In another aspect, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### II. Obtaining Encoded Polynucleotide and Polypeptide Aspects

In some aspects, there are nucleic acid molecule encoding enzyme polypeptides, such as an FTO protein. These may be generated by methods known in the art, e.g., expressed in any suitable recombinant expression system and allowed to assemble to form the FTO molecules.

### A. Expression

The nucleic acid molecules may be used to express large quantities of other nucleic acids, such as mRNAs, and/or polypeptides. In some aspects, nucleic acids are employed to generate large quantities of mRNA encoding an FTO protein, which are then purified for use in methods described herein. In some aspects, nucleic acids are employed to generate large quantities of an FTO protein, which are then purified for use in methods described herein.

### 1. Vectors

In some aspects, contemplated are expression vectors comprising a nucleic acid molecule encoding a polypeptide of the desired sequence or a portion thereof. In some aspects, expression vectors comprising nucleic acid molecules may encode fusion proteins, modified proteins, and probes thereof. In addition to control sequences that govern transcription and translation, vectors and expression vectors may contain nucleic acid sequences that serve other functions as well.

To express the proteins or fragments thereof, DNAs encoding partial or full-length proteins, such as partial or full-length FTO, are inserted into expression vectors such that the gene area is operatively linked to transcriptional and translational control sequences. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### 2. Expression Systems

Numerous expression systems exist that comprise at least a part or all of the expression vectors discussed above. Prokaryote- and/or eukaryote-based systems can be employed for use with an aspect to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### 3. Methods of Gene Transfer

Suitable methods for nucleic acid delivery to effect expression of gene products, such as an FTO protein, are contemplated to include virtually any method by which a nucleic acid (e.g., DNA, including viral and nonviral vectors) can be introduced into a cell, a tissue, or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### 4. Host Cells

In another aspect, contemplated are the use of host cells into which a recombinant expression vector has been introduced. Proteins and mRNAs, such as an FTO protein and/or mRNA, can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts. The nucleic acid molecule encoding an FTO protein may be isolated from any source that produces the protein.

### III. Kits

Certain aspects of the present disclosure also concern kits containing compositions of the disclosure or compositions to implement methods disclosed herein. In some aspects, kits can be used to evaluate one or more biomarkers. In certain aspects, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein.

Kits may comprise components, which may be individually packaged or placed in a container, such as a tube, bottle, vial, syringe, or other suitable container means.

Individual components may also be provided in a kit in concentrated amounts; in some aspects, a component is provided individually in the same concentration as it would be in a solution with other components. Concentrations of components may be provided as 1×, 2×, 5×, 10×, or 20× or more.

It is contemplated that any method or composition described herein can be implemented with respect to any other method or composition described herein and that different aspects may be combined. The claims originally filed are contemplated to cover claims that are multiply dependent on any filed claim or combination of filed claims.

### IV. Formulations and Culture of the Cells

In particular aspects, the cells of the disclosure, such as fertilized cells, may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain aspects, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). In specific aspects, one or more of these may be explicitly excluded.

In some aspects, the medium further comprises vitamins. In some aspects, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some aspects, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some aspects, the vitamins include or consist essentially of biotin, DL alpha tocopherol acetate, DL alpha-tocopherol, vitamin A, or combinations or salts thereof. In some aspects, the medium further comprises proteins. In some aspects, the proteins comprise albumin or bovine serum albumin, a fraction of BSA, catalase, insulin, transferrin, superoxide dismutase, or combinations thereof. In some aspects, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some aspects, the medium comprises one or more of the following: a B-27® supplement, xeno-free B-27® supplement, GS21TM supplement, or combinations thereof. In some aspects, the medium comprises or further comprises amino acids, monosaccharides, inorganic ions. In some aspects, the amino acids comprise arginine, cystine, isoleucine, leucine, lysine, methionine, glutamine, phenylalanine, threonine, tryptophan, histidine, tyrosine, or valine, or combinations thereof. In some aspects, the inorganic ions comprise sodium, potassium, calcium, magnesium, nitrogen, or phosphorus, or combinations or salts thereof. In some aspects, the medium further comprises one or more of the following: molybdenum, vanadium, iron, zinc, selenium, copper, or manganese, or combinations thereof. In certain aspects, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21TM supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific aspects, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific aspects, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific aspects, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular aspects the cells are in a formulated cell suspension that is stable at room temperature for 1, 2, 3, or 4 hours or more from time of thawing.

### A. Cells

Certain aspects relate to cells comprising polypeptides or nucleic acids of the disclosure, including FTO. In some aspects, the cell is a stem cell and/or cancer cell. In some aspects the cell is a fertilized cell, such as a zygote, blastocyst, or embryo cell. In some aspects, the cell is a gamete. The cells may be introduced exogenous FTO or an FTO inhibitor.

Suitable mammalian cells include primary cells and immortalized cell lines. Suitable mammalian cell lines include human cell lines, non-human primate cell lines, rodent (e.g., mouse, rat) cell lines, bovine cell lines, pig cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual, including a livestock animal. For example, in some cases, the cell is an immune cell obtained from an individual. As an example, the cell is a T lymphocyte obtained from an individual. As another example, the cell is a cytotoxic cell obtained from an individual. As another example, the cell is a stem cell (e.g., peripheral blood stem cell) or progenitor cell obtained from an individual.

### EXAMPLES

The following examples are included to demonstrate preferred aspects of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the invention.

LINE1 RNA is A Major Substrate of FTO in mESCs

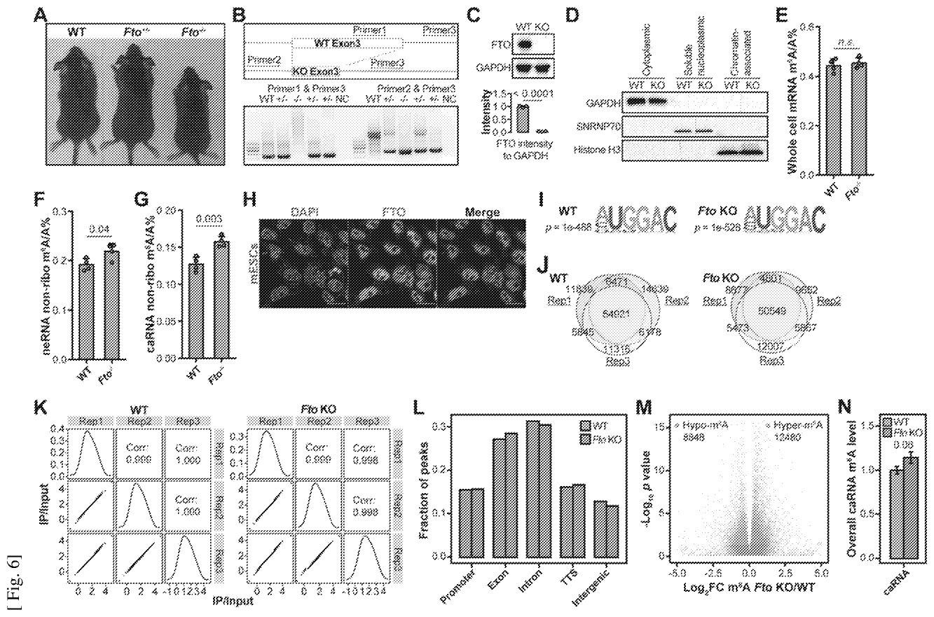

To uncover the major substrates of FTO in certain contexts, the inventors derived Fto^{-/-} and control wild-type (WT) mESCs (FIGS. 6A to 6C). The inventors quantified m⁶A level changes of RNAs isolated from different subcellular fractions between Fto^{-/-} and WT mESCs (FIG. 6D). The m⁶A level of RNA isolated from chromatin-associated and soluble nuclear fractions was increased (FIGS. 6E to 6G), consistent with the nuclear localization of FTO (FIG. 6H). The inventors performed m⁶A MeRIP-seq to examine the chromatin-associated RNA (caRNA) methylome of WT and Fto^{-/-} mESCs (FIGS. 61 to 6L) and detected more hypermethylated peaks with Fto depletion (FIG. 6M), accompanied by an increased overall caRNA m⁶A level (FIG. 6N).

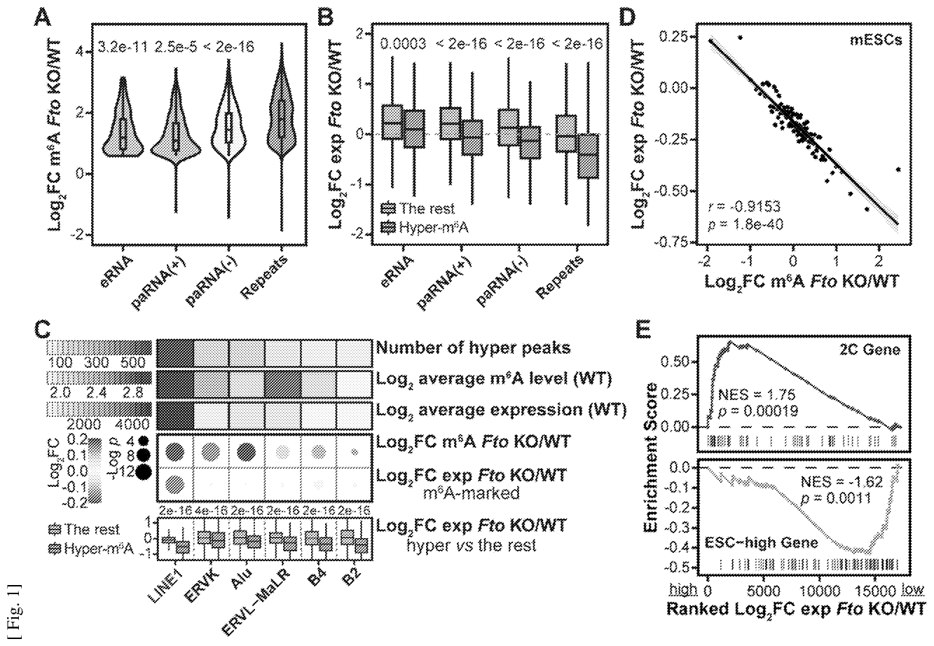

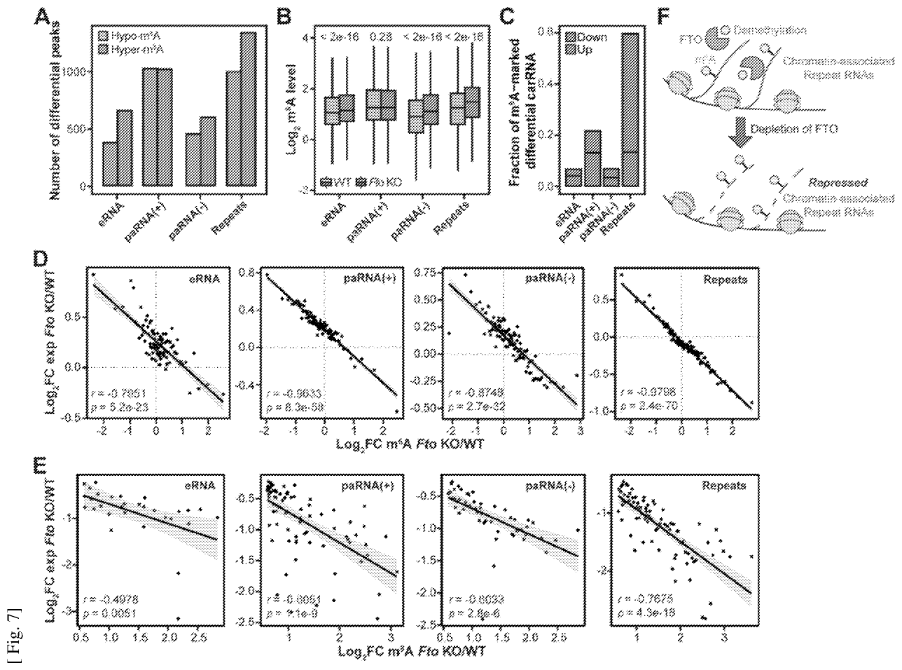

The inventors annotated carRNAs as promoter-associated RNA (paRNA), enhancer RNA (eRNA), and RNA transcribed from transposable elements (repeat RNA) (22). Most carRNAs exhibited more hypermethylated m⁶A peaks (FIG. 7A) and elevated m⁶A levels (FIG. 7B) upon Fto KO. Compared to other carRNAs, Fto depletion led to more pronounced hypermethylation of repeat RNAs (FIG. 1A), more downregulated m⁶A-marked repeat RNAs (FIG. 7C), and greater downregulation of hypermethylated repeat RNAs (FIG. 1B). Upon Fto KO, carRNA expression changes negatively correlated with their m⁶A level changes (FIG. 7D), with repeat RNAs showing the strongest correlation between transcript downregulation and m⁶A hypermethylation (FIGS. 7E and 7F).

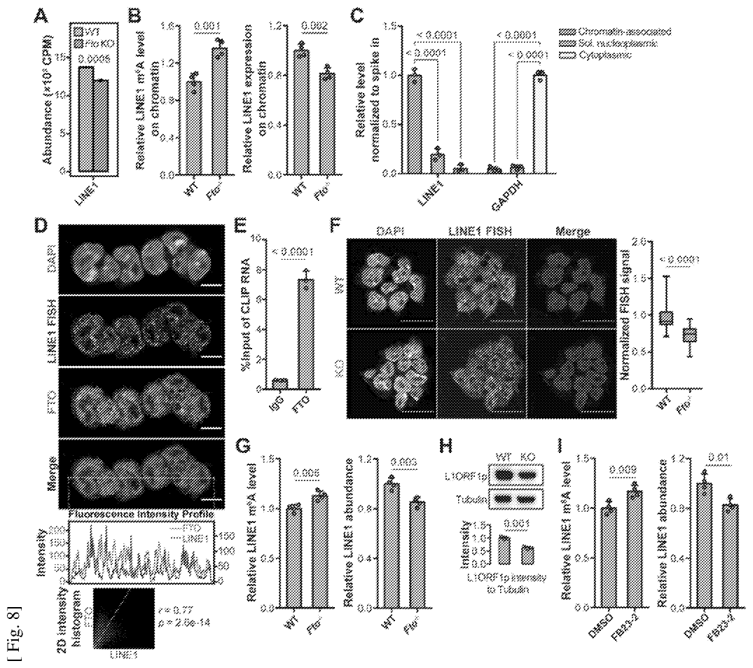

Among m⁶A-marked repeat RNAs, long-interspersed element-1 (LINE1) RNA emerged as a major substrate of FTO in mESCs; it showed the highest number of hypermethylated peaks, most increased m⁶A levels, most reduced abundance (FIG. 1C), and a reduced overall expression (FIGS. 8A and 8B) upon Fto KO. LINE1 RNA mainly associates with chromatin in mESCs (FIGS. 8C and 8D) (26, 27). The inventors observed colocalization of LINE1 RNA and FTO protein (FIG. 8D) and validated the binding of LINE1 RNA by FTO (FIG. 8E). The m⁶A levels and expressions of whole-cell LINE1 RNA exhibited similar changes to those on chromatin upon Fto KO (FIGS. 8F and 8G), accompanied by reduced L1ORF1p expression (FIG. 8H). Treating WT mESCs with an FTO inhibitor (9) recapitulated the effect of Fto KO on LINE1 RNA (FIG. 8I).

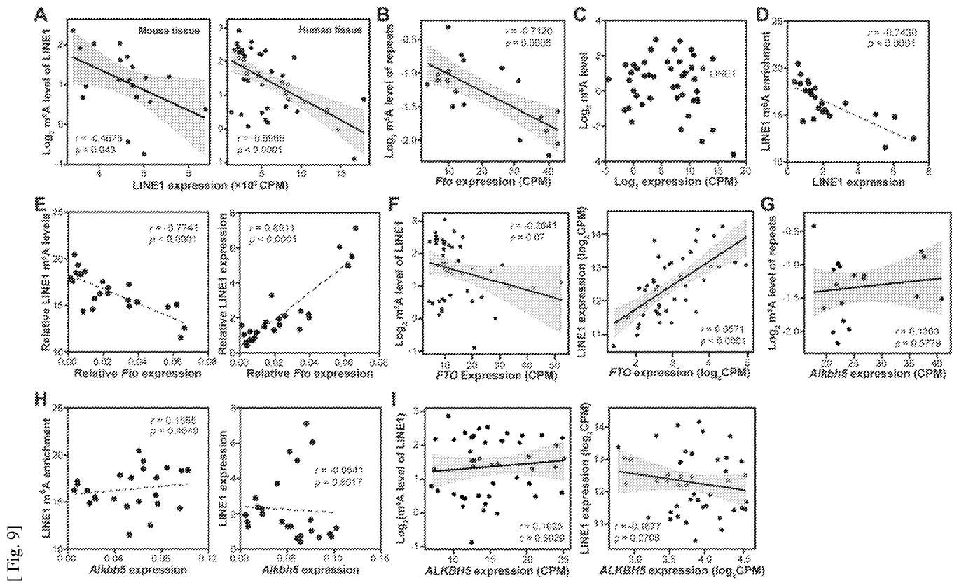

Upon Fto KO, the inventors observed that greater m⁶A level increases correlate with greater LINE1 RNA abundance reductions (FIG. 1D). Across published mouse and human tissue m⁶A methylomes (13), LINE1 RNA m⁶A level also negatively correlated with its expression, and high FTO expression was associated with low m⁶A level and high expression of LINE1 RNA (FIG. 9), supporting LINE1 RNA as a substrate of FTO in most tissues.

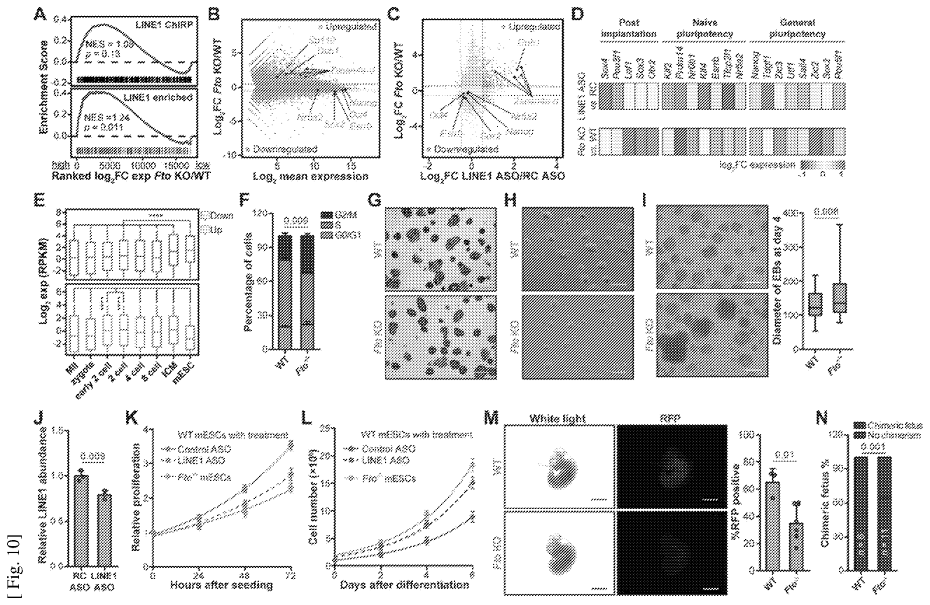

LINE1 elements are one of the most abundant retrotransposons in mammalian genomes, and LINE1 RNA is known to play critical roles during mammalian early development (26, 27). In mESCs, LINE1 RNA can function as a nuclear RNA scaffold for trans-regulation, with LINE1 RNA knockdown by morpholino antisense oligo (ASO) causing activated two-cell (2C) program and repressed ESC-high genes (27, 28). Fto KO largely recapitulated these transcriptomic changes (FIG. 1E), with lower 2C gene activation but greater downregulation of ESC-high genes (FIG. 10A to 10E). Fto KO also caused several similar phenotypic changes to LINE1 ASO treatment in mESCs, including cell cycle dysregulation, self-renewal impairment, and induction of capacity to form embryoid bodies (EBs) (FIGS. 10F to L). Moreover, Fto^{-/-} mESCs exhibited a reduced ability to integrate to chimeric mice compared to WT mESCs (FIGS. 10M and 10N).

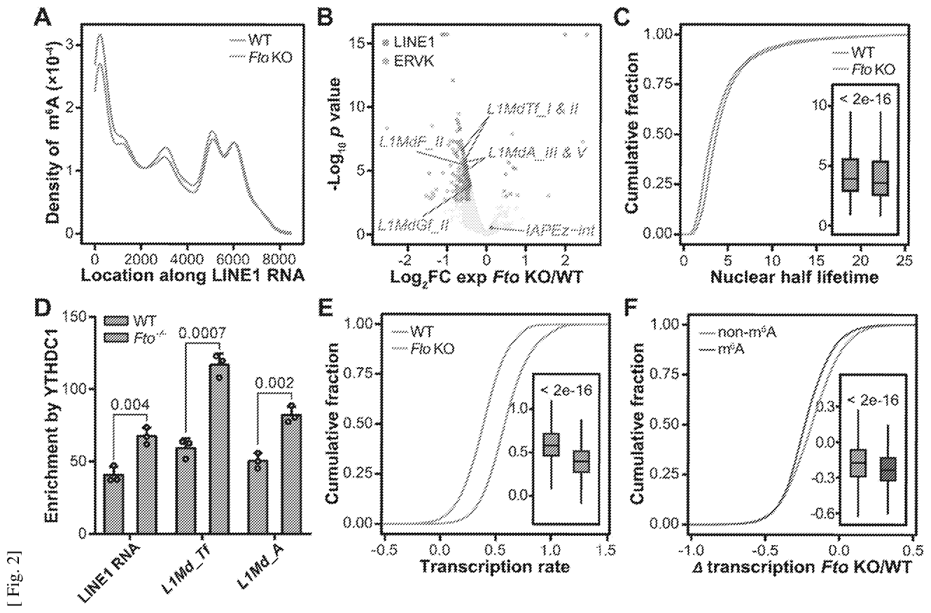

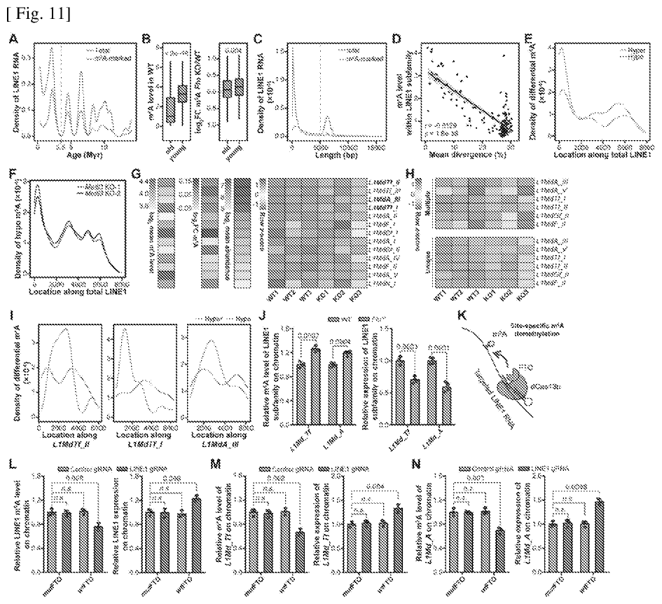

Evolutionarily young LINE1s display higher RNA m⁶A levels and greater m⁶A increases upon Fto KO (FIGS. 11A and 11B). Longer and less divergent LINE1 RNA also tends to exhibit a higher m⁶A level (FIGS. 11C and 11D). The inventors observed elevated m⁶A density near the 5′-end with Fto depletion (FIG. 2A, and FIG. 11E), which also responds to Mettl3 KO (FIG. 11F). the analysis suggested that several young LINE1 subfamilies were downregulated and hypermethylated upon Fto KO (FIG. 2B and FIGS. 11G to 11J). The inventors employed a CRISPR dCas13b system fused with WT FTO or a catalytically inactive mutant (FIG. 11K) (22) and observed that delivery of dCas13b-wtFTO by guide RNA (gRNA) targeting LINE1 RNA reversed its m⁶A level and expression changes (FIGS. 11L to 11N).

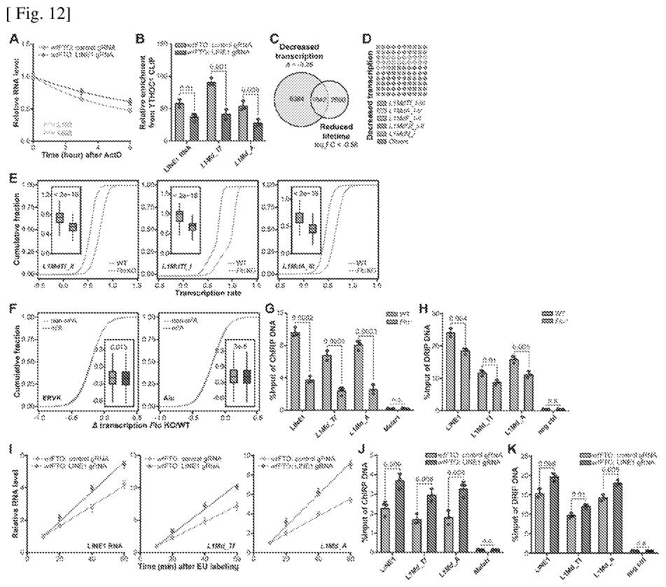

Consistent with the reported YTHDC1-mediated destabilization of m⁶A-marked carRNAs (22), the inventors detected accelerated decay of LINE1 RNA upon Fto KO (FIG. 2C), accompanied by elevated binding of LINE1 RNA by YTHDC1 (FIG. 2D). Both changes were recovered by targeting LINE1 RNA with dCas13b-wtFTO in Fto^{-/-} mESCs (FIGS. 12A and 12B).

LINE1 RNA transcription was markedly reduced with Fto depletion (FIG. 2E and FIGS. 12C to 12E). Moreover, Fto KO led to greater decreases in transcription rates of m⁶A-marked versus unmarked LINE1 RNAs (FIG. 2F) but not ERVK or Alu transcripts (FIG. 12F). The inventors also observed reduced DNA association of LINE1 RNA and decreased R-loop formation around LINE1 loci with Fto depletion (FIGS. 12G and 12H). These effects caused by Fto KO could all be reversed by targeting dCas13b-wfFTO to LINE1 RNA (FIGS. 12I to 12K). Therefore, FTO appears to mediate m⁶A demethylation of a subset of LINE1 RNA, maintaining their levels on chromatin.

Fto KO Leads to Closed Chromatin in mESCs

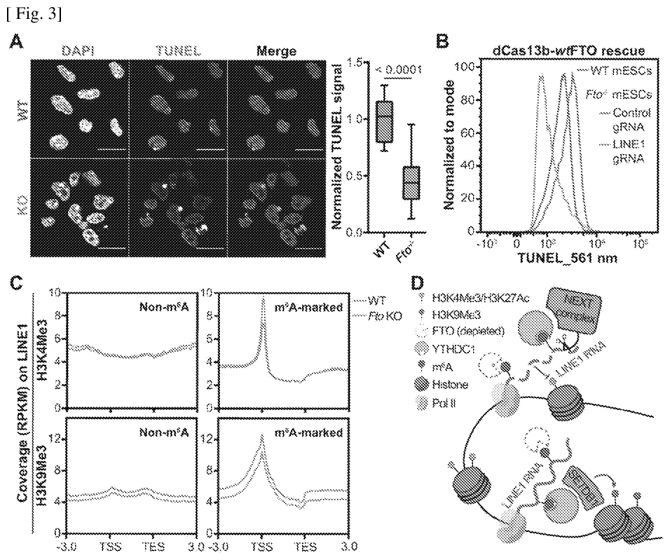

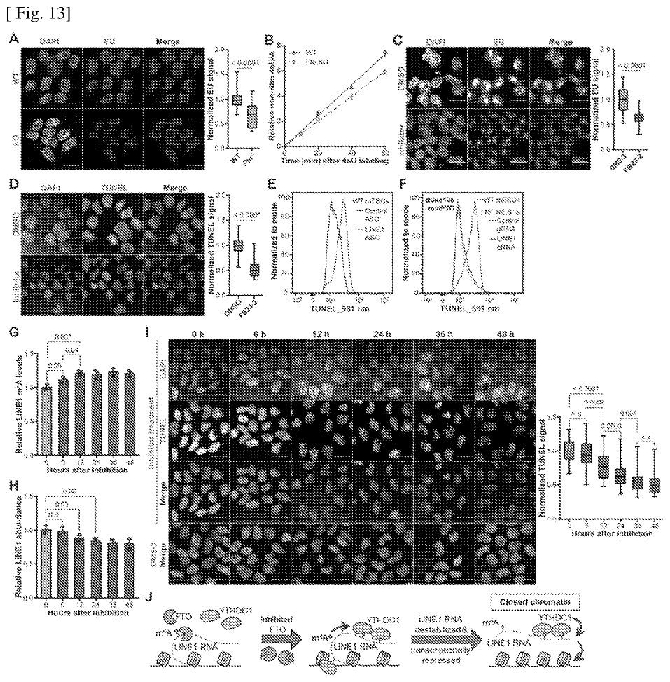

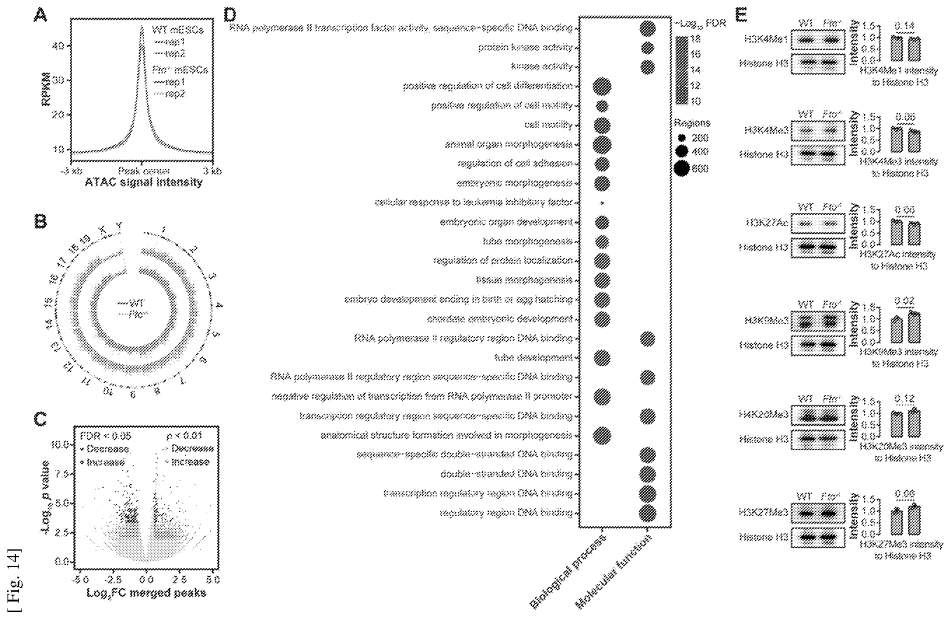

LINE1 RNA and m⁶A on carRNAs have been shown to regulate chromatin state and transcription (22-27). Indeed, the inventors observed decreased nascent RNA synthesis (FIGS. 13A and 13B) accompanied by more closed chromatin (FIG. 3A) upon Fto KO; similar effects were observed when treating WT mESCs with an FTO inhibitor (FIGS. 13C and 13D). Additionally, LINE1 ASO treatment in WT mESCs also led to more closed chromatin (FIG. 13E), while delivering dCas13b-wtFTO to LINE1 RNA largely rescued chromatin closure observed in Fto^{-/-} mESCs (FIG. 3B and FIG. 13F). The inventors next monitored LINE1 RNA m⁶A demethylation and chromatin accessibility in a time-course FTO inhibition in WT mESCs (FIGS. 13G to 13J). ATAC-seq results also validated reduced chromatin accessibility upon Fto KO (FIGS. 14A to 14C), with gained-closed regions enriching gene ontology (GO) terms relevant to development (FIG. 14D). Slightly decreased H3K4Me3 and H3K27Ac, and increased H3K9Me3 and H3K27Me3 were observed with Fto depletion (FIG. 14E).

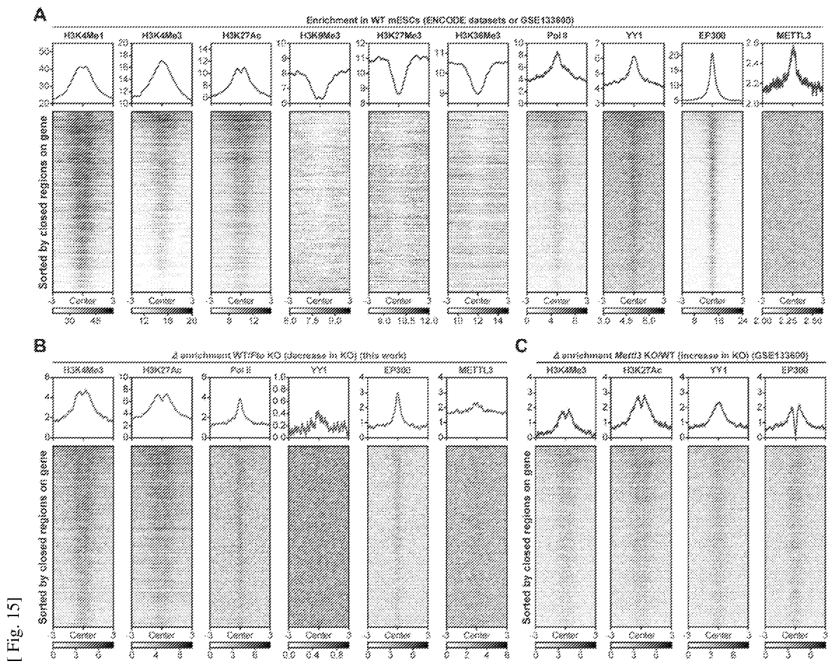

YY1 and EP300 can be recruited by caRNA to promote transcription (22, 29, 30). The inventors found notable enrichment at gained-closed regions caused by Fto KO for H3K4Me1, H3K4Me3, and H3K27Ac, as well as YY1, EP300, and Pol II binding, but not repressive histone marks (FIG. 15A); consistently, ChIP-seq experiments confirmed reduced chromatin accessibility of these regions upon Fto depletion (FIG. 15B). Fto KO-induced gained-closed regions are also affected by Mettl3 KO (FIG. 15C) (22). In turn, chromatin closure upon Fto KO could reduce access to METTL3, potentially explaining m⁶A hypomethylation at certain genomic regions.

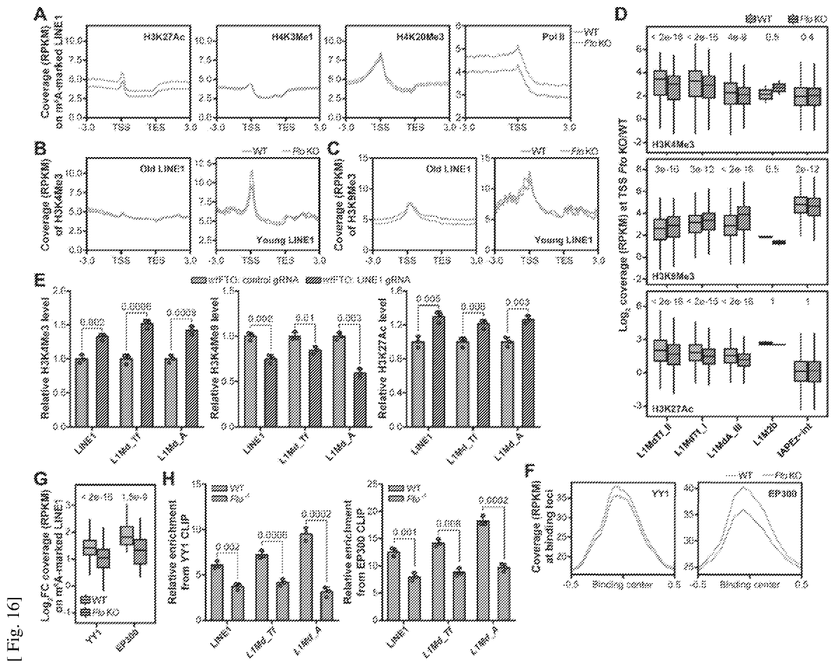

The inventors noticed distinct profiles of H3K4Me3 and H3K9Me3 between loci with m⁶A-marked and unmarked LINE1 RNA (FIG. 3C). At m⁶A-marked LINE1 RNA loci, the inventors observed decreased H3K4Me3 and H3K27Ac levels and Pol II binding upon Fto KO, accompanied by increased H3K9Me3 levels (FIG. 3C and FIGS. 16A to 16C). Similar patterns were observed for FTO-targeted LINE1 subfamilies but not L1M2b, an unmarked LINE1 subfamily, or IAPEz-int, an ERVK subfamily regulated by METTL3 (23-25) (FIG. 16D). Targeting LINE1 RNA with dCas13b-wtFTO reversed the dysregulated histone marks (FIG. 16E). Moreover, Fto KO led to decreased chromatin association and LINE1 RNA binding of YY1 and EP300 (FIGS. 16F to 16H).

Altogether, the data reveal an interplay between LINE1 RNA m⁶A demethylation by FTO and chromatin state. After Fto depletion, increased m⁶A on LINE1 RNA could promote YTHDC1 binding, which could recruit nuclear decay machinery (22) and histone modifiers that add repressive histone marks (23, 24), resulting in reduced LINE1 RNA abundance and more closed chromatin (FIG. 3D).

### Fto Regulates Gene Expression Mostly Through Intragenic LINE1 RNA

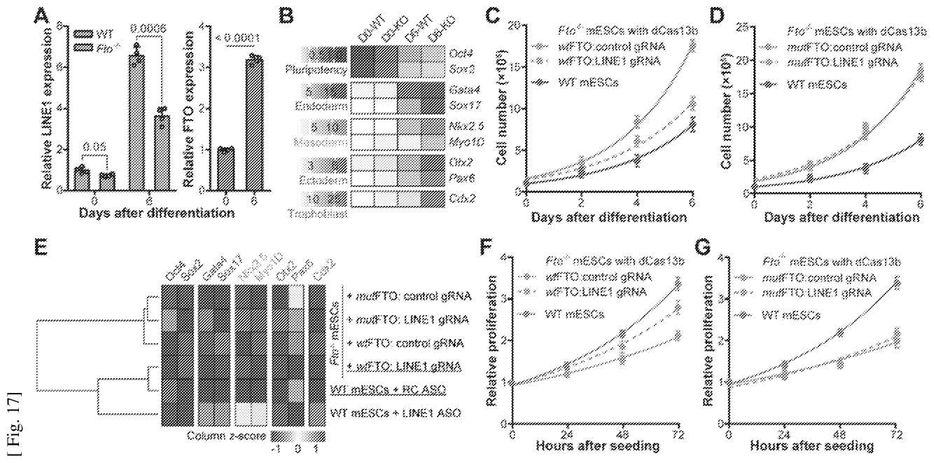

The inventors observed that Fto and LINE1 RNA abundance increased after EB differentiation induction, but LINE1 RNA increase was diminished upon Fto KO (FIG. 17A). Targeting dCas13b-wtFTO to LINE1 RNA in Fto^{-/-} mESCs partially rescued aberrantly expressed differentiation markers associated with induced EB differentiation and impaired self-renewal (FIGS. 17B to 17G).

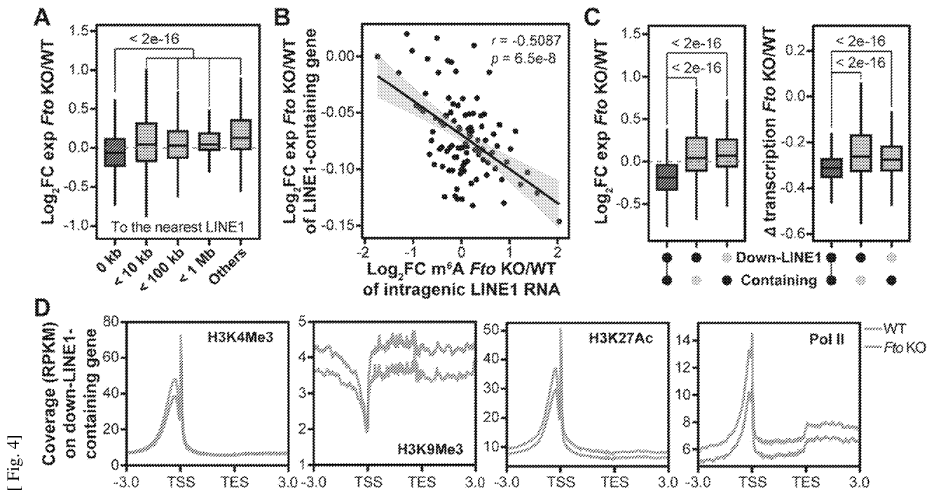

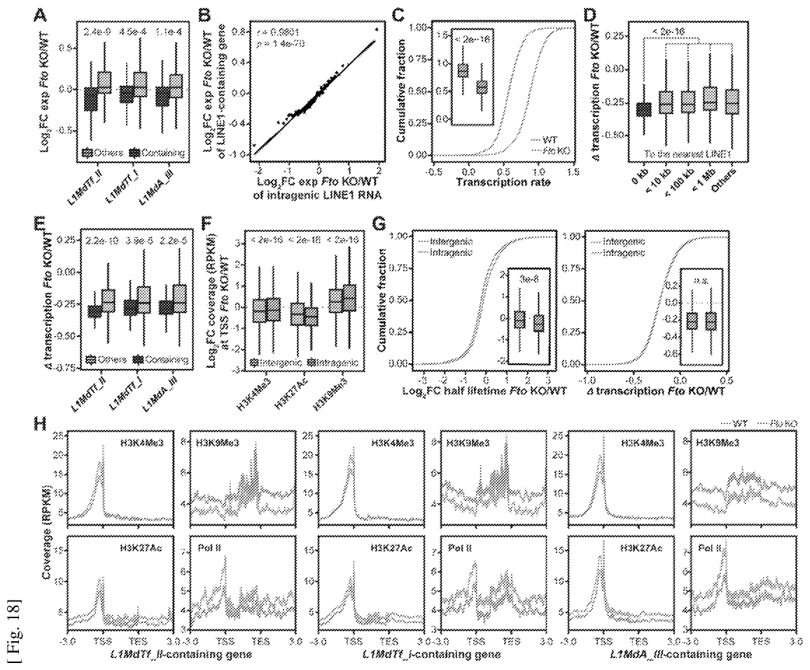

Thus, the inventors investigated the mechanism underlying the impact of Fto KO on mESC differentiation. The inventors found that genes that contain LINE1 RNA (termed as LINE1-containing genes) were downregulated upon Fto KO compared to genes beyond 10 kb from LINE1 RNA (FIG. 4A and FIG. 18A) (26). The expression of LINE1-containing genes and intragenic LINE1 RNA tended to decrease with Fto depletion, accompanied by increased intragenic LINE RNA m⁶A levels (FIG. 4B and FIG. 18B). LINE1-containing genes also displayed greater transcription rate reductions compared to other genes upon Fto KO (FIGS. 18C to 14E).

Both intragenic and intergenic LINE1 RNA showed suppressed expression upon Fto KO, but intragenic ones were generally more responsive (FIGS. F and G). The inventors further explored the cis-regulatory role of intragenic LINE1 RNAs suppressed by Fto KO and the inventors noticed significantly decreased expression and reduced transcription for genes that contain downregulated LINE1 RNA compared to other genes (FIG. 4C). Accordingly, reduced H3K4Me3 and H3K27Ac levels and Pol II binding, accompanied by elevated H3K9Me3 levels, were observed for these gene loci (FIG. 4D) and gene loci that contain FTO-targeted LINE1 subfamilies (FIG. 18H).

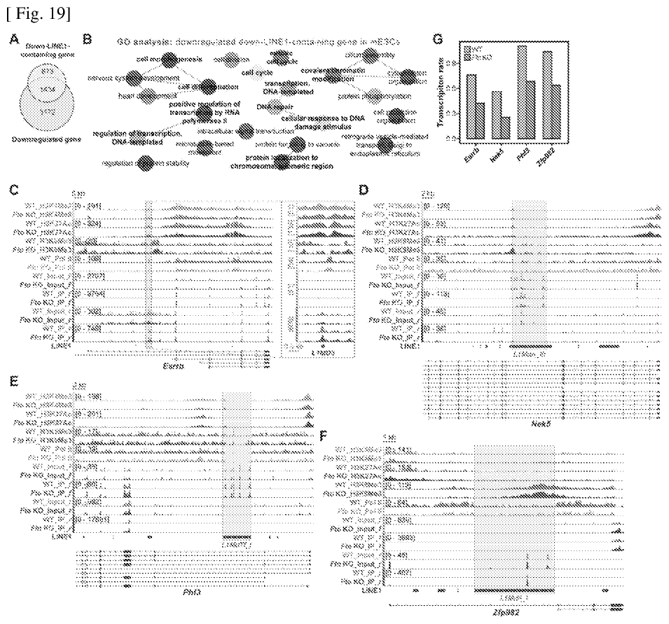

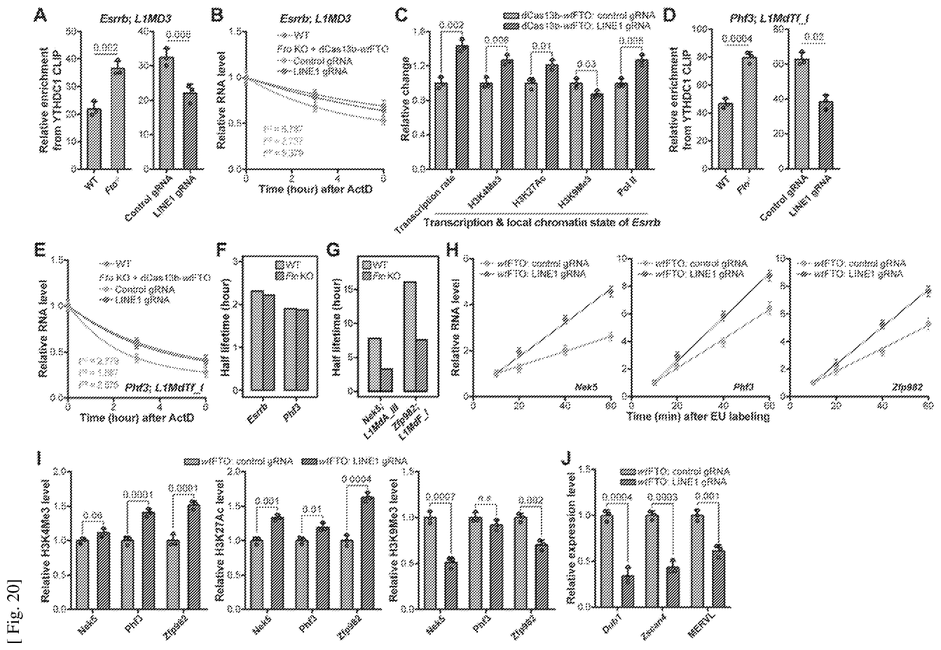

Most genes that contain downregulated LINE1 RNA were also downregulated upon Fto KO (FIG. 19A), and these genes enriched pathways involved in differentiation and development (FIG. 19B). The inventors examined several key genes in ESC pluripotency and early development (Esrrb, Nek5, Phf3, and Zfp982) and found that all of them contain intragenic LINE1 RNA with increased m⁶A level and decreased expression upon Fto KO, associated with more closed local chromatin and reduced gene transcription rates (FIGS. 19C to 19G), which could all be reversed by targeting LINE1 RNA with dCas13b-wtFTO (FIGS. 20A to 20I).

Notably, certain 2C genes such as Dub1 and Zscan4 do not contain LINE1 RNA. These genes and MERVL RNA, a 2C-specific transposon, could be repressed by LINE1 RNA through trans-regulation (27). For these transcripts, the inventors observed reduced expression when applying dCas13b-wtFTO to LINE1 RNA in Fto^{-/-} mESCs (FIG. 20J).

### The FTO-LINE1 RNA Axis is Functionally Relevant in Mouse Development

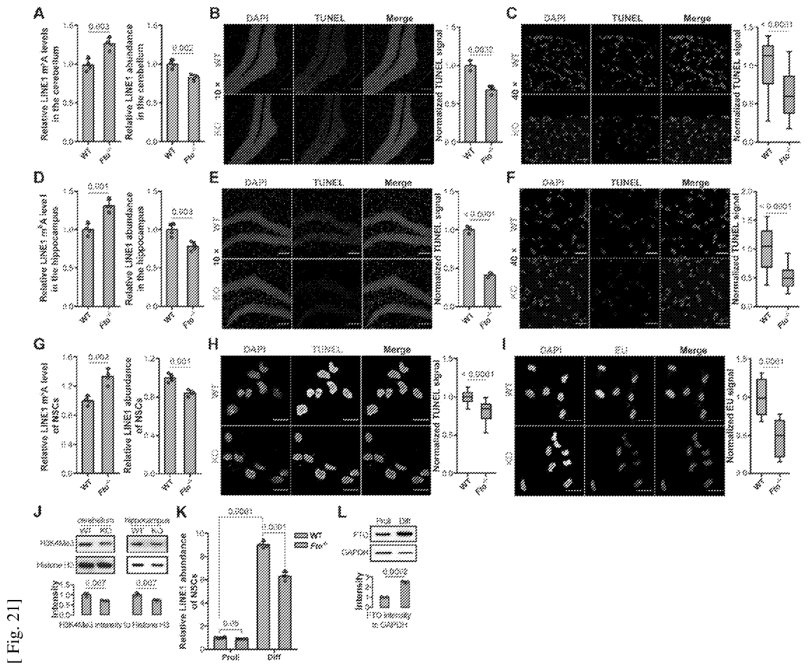

The inventors examined the mouse cerebellum, hippocampus, and adult neural stem cells (aNSCs) since Fto is highly expressed in mouse brain tissues (31). The inventors observed increased LINE1 RNA m⁶A levels, decreased LINE1 RNA expression, and more closed chromatin from samples derived from Fto^{-/-} mice compared to WT controls (FIGS. 21A to 21J). Similar trends of Fto and LINE1 RNA abundance during mESC differentiation were also observed for aNSCs (FIGS. 21K and 21L).

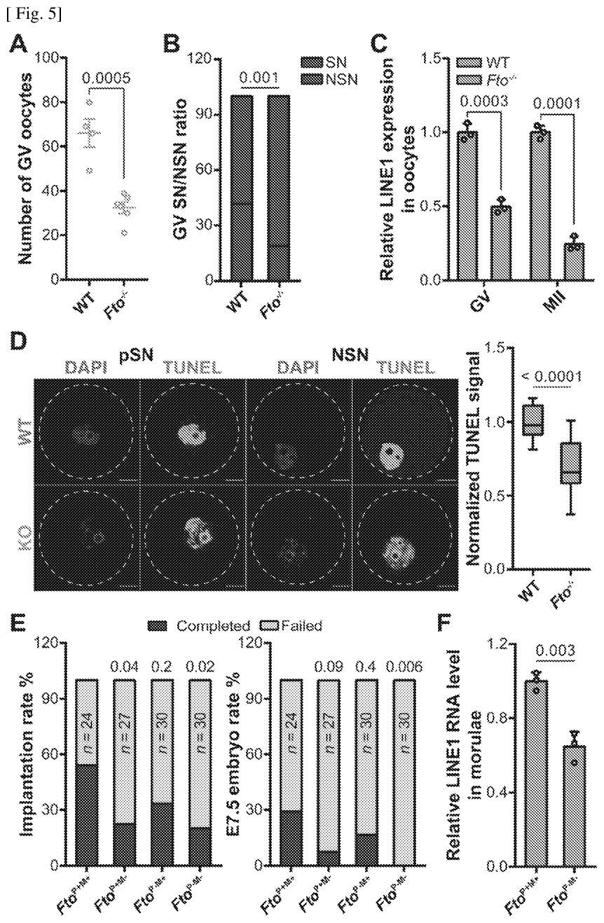

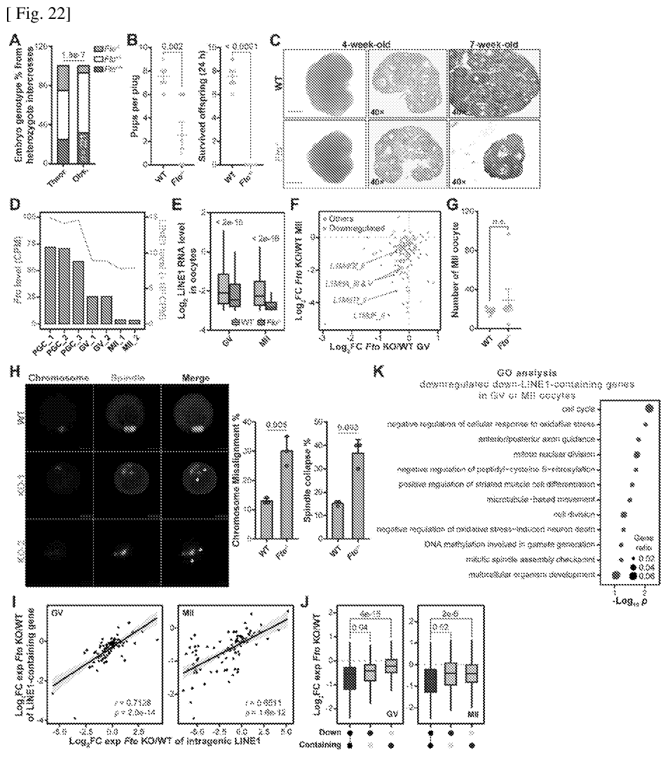

The inventors next investigated the effects of FTO during early development. Fto^{-/-} pups were born at a lower rate than the expected Mendelian ratio (FIG. 22A). Fto^{-/-} female mice showed ovarian defects and impaired fertility (FIGS. 22B and 22C). Previous studies uncovered that LINE1 is activated before the sex determination of primordial germ cells (PGCs) and the meiotic entry of oocytes (32, 33). Indeed, both Fto and LINE1 RNA expression decreased from PGC to the metaphase II (MII) stage (FIG. 22D). The number of germinal vesicle (GV) oocytes and ratio of surrounded nucleolus (SN) GV oocytes from Fto^{-/-} mice were lower compared to WT controls (FIGS. 5A and 5B). Fto depletion led to significantly reduced LINE1 RNA expression in GV and MII oocytes (FIG. 5C and FIG. 22E), with downregulated LINE1 subfamilies largely resembling those observed in mESCs (FIG. 22F). The inventors noticed more closed chromatin for GV oocytes from Fto^{-/-} mice compared to WT controls (FIG. 5D). Fto^{-/-} GV could mature to the MII stage (FIG. 22G), but Fto^{-/-} MII oocytes showed increased chromosome misalignment and spindle collapse (FIG. 22H). RNA-seq results revealed greater expression decreases of genes that contain downregulated LINE1 RNA compared to other genes in Fto^{-/-} GV and MII oocytes, respectively (FIGS. 221 and 22J). GO analysis suggests that Fto KO causes observed defects in oocyte development likely through LINE1 RNA which regulates LINE1-containing genes (FIG. 22K).

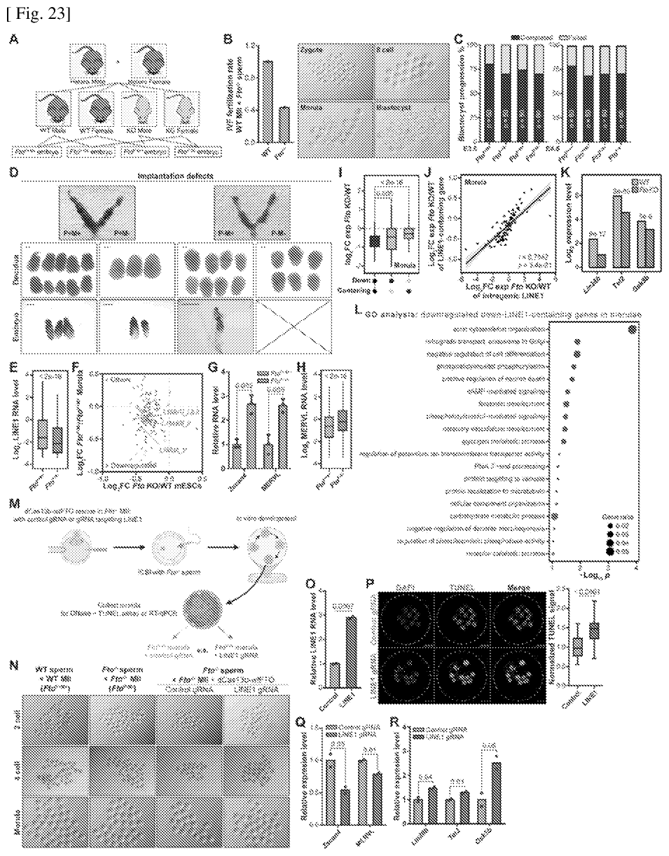

The inventors further studied WT (Fto^{P+/M+}), maternal Fto-deficient (Fto^{P+/M-}), paternal Fto-deficient (Fto^{P-/M+}), and homozygous KO (Fto^{P-/M-}) embryos (FIGS. 23A and 23B). Embryos from all four groups could reach the blastocyst stage (E3.5) and hatch out of the zona pellucida at E4.5, but Fto-deficient embryos showed a slightly weakened ability to do so (FIG. 23C). Moreover, maternal loss of Fto severely impeded decidua formation and the generation of E7.5 embryos, with no E7.5 embryos produced upon homozygous Fto depletion (FIG. 5E and FIG. 23D). The inventors examined the transcriptome differences between Fto^{P-/M-} and Fto^{P+/M+} morulae, where the inventors detected repressed LINE1 RNA and downregulated LINE1 subfamilies similar to those in mESCs (FIG. 5F, and FIGS. 23E and 23F). The inventors again observed induced expression of Zscan4 and MERVL RNA (FIGS. 23G and 23H) and greater expression decreases of genes that contain downregulated LINE1 RNA, including regulators essential during early embryonic development such as Lin28b, Tet2, and Gsk3b (FIGS. 23I to 23L).

The inventors last applied dCas13b-wtFTO in Fto^{-/-} MII oocytes and fertilized them with Fto^{-/-} sperm; embryos were developed in vitro to the morula stage for subsequent analyses (FIGS. 23M and 23N). Induced LINE1 RNA associated with more open chromatin was observed in Fto^{P-/M-} morulae by targeting dCas13b-wtFTO to LINE1 RNA in Fto^{-/-} MII oocytes (FIGS. 23O and 23P). The expression of Zscan4, MERVL RNA, and selected genes that contain downregulated LINE1 RNA were also reversed in the rescued embryos (FIGS. 23Q and 23R).

Correlations Between FTO, METTL3 and YTHDC1 in mESCs

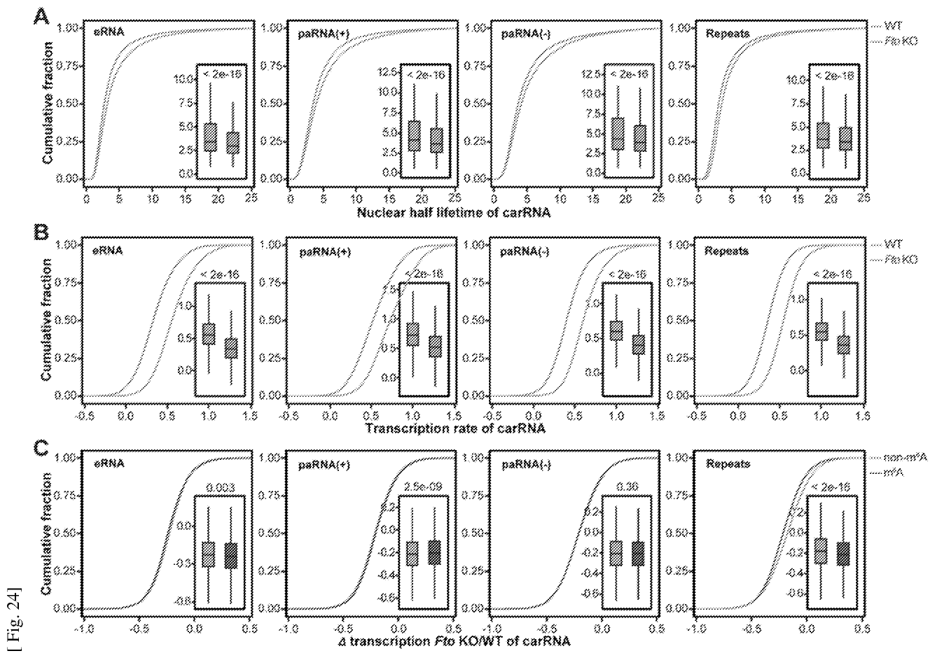

Similar to the observations for LINE1 RNA, the inventors detected both decreased lifetimes and transcription rates for all three types of carRNAs upon Fto KO in mESCs (FIGS. 24A and 24B). However, only repeat RNAs showed an obviously greater decrease in the change in transcription rates upon Fto KO for m⁶A-marked repeat RNAs compared to their unmethylated counterparts (FIG. 24C). This could largely be due to the prominent impacts of Fto KO on LINE1 RNA (FIG. 2F) but not on other repeat families with relatively high m⁶A levels like Alu or ERVK (FIG. 12F).

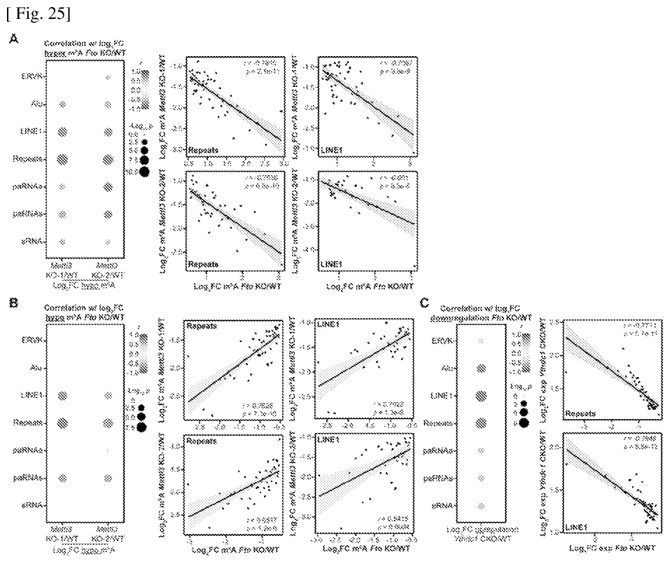

For most carRNAs and selected repeat RNAs, the inventors noticed distinct negative correlations between m⁶A level changes of Fto KO-induced hypermethylation and Mettl3 KO-induced hypomethylation, with LINE1 RNA showing strong correlations (FIG. 25A). Notably, positive correlations were also observed between m⁶A level changes of Fto KO- and Mettl3 KO-caused hypomethylated m⁶A peaks for LINE1 RNA (FIG. 25B). This relates to the observation that m⁶A hypomethylation upon Fto KO correlates with METTL3-sensitive peaks at the middle region of LINE1 RNA (FIGS. 25E and 25F). Together, these findings support the idea that decreased levels of some m⁶A sites upon Fto KO could be due to more closed chromatin and impaired access of METTL3 to its substrates (FIGS. 13G to 13J, and FIGS. 15A and 15B). Future experiments could test the kinetics of these hypomethylated peaks versus hypermethylated ones upon FTO inhibition in mESCs.

The inventors also found negative correlations between expression changes of Fto KO-induced downregulated transcripts and Ythdc1 cKO-induced upregulated transcripts (FIG. 25C), again with LINE1 RNA showing the strongest correlations. These results are consistent with a mechanistic connection between the two proteins: lower FTO leads to more m⁶A methylation on LINE1 RNA, recruiting more YTHDC1, and in turn repressing the transcript levels. Notably, m⁶A levels and expression changes of repeat RNAs and LINE1 RNA exhibited stronger correlations between Fto KO and Mettl3 KO or Ythdc1 cKO compared to other carRNAs and other repeat families, respectively, supporting LINE1 RNA as a major substrate of FTO-mediated m⁶A demethylation in mESCs.

Context-Dependent Chromatin State Regulation by FTO Targeting LINE1 RNA and mRNA

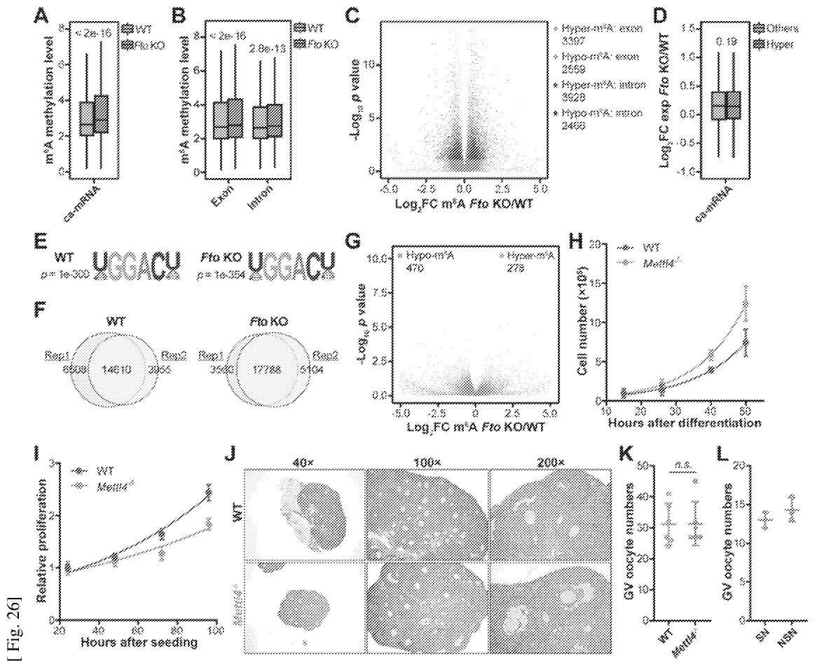

In mESCs, chromatin-associated mRNA (ca-mRNA) displayed increased m⁶A levels in both introns and exons upon Fto KO, accompanied by more hypermethylated m⁶A peaks (FIGS. 26A to 26C). However, the inventors only found minimal expression changes between hypermethylated and non-hypermethylated ca-mRNAs (FIG. 26D), indicating that FTO may not directly affect ca-mRNA expression through m⁶A demethylation. In contrast, Fto depletion led to only minor changes in the m⁶A methylome of whole-cell polyadenylated RNA (FIGS. 26E to 26G), supporting the dominant nuclear role of FTO when localized in the cell nucleus (16).

mRNA cap-m⁶Aₘ demethylation by FTO is mostly a cytoplasmic event (16, 60), and depletion of the corresponding methyltransferase does not affect mouse viability or fertility (20), excluding a critical role for this process in mouse early development. In the cell nucleus, FTO also demethylates m⁶Aₘ on certain snRNAs (15, 16), of which METTL4 has been identified as the methyltransferase (61). If FTO were to regulate pluripotency and self-renewal of mESCs through snRNA m⁶Aₘ demethylation, a reduced mEB formation capacity and induced proliferation, opposite to the effects of Fto KO, would be expected for Mettl4 depletion. On the contrary, the inventors observed induced differentiation (FIG. 26H) and reduced proliferation (FIG. 26I) upon Mettl4 KO. METTL4 was also shown not to affect mouse viability (62). The studies confirmed minimal effects of Mettl4 KO on mouse oogenesis (FIGS. 26J to 26L). These data in mESCs and mouse oocytes suggested that snRNA m⁶Aₘ is not a substrate of FTO that functionally affects mouse early development.

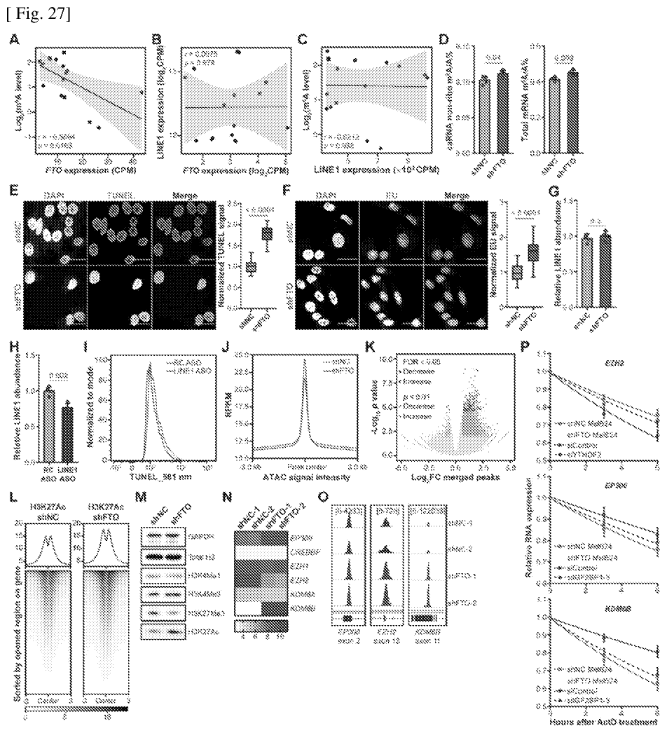

Previous work has shown that polyadenylated RNA m⁶A demethylation by FTO plays critical roles in several human cancer cell lines (7-12). The inventors speculated that FTO may play different roles depending on cellular context. Although LINE1 RNA m⁶A level negatively correlates with FTO expression in selected human cancer cells (13), LINE1 RNA expression does not (FIGS. 27A and 27B), and there is no correlation between LINE1 m⁶A level and expression (FIG. 27C).

The inventors performed further investigations in Mel624 melanoma cells, since the inventors found that FTO knockdown sensitizes their response to anti-PD-L1 blockade (63). Unlike the observations from mESCs, FTO knockdown in Mel624 cells led to increased m⁶A levels in both caRNA and mRNA (FIG. 27D). A numerically greater increase in m⁶A was detected for mRNA since the absolute m⁶A level (shown as the m⁶A/A ratio) in mRNA was ˜4-fold higher than that in caRNA with rRNA depletion. To the surprise, FTO knockdown in Mel624 cells led to more open chromatin (FIG. 27E) and induced nascent RNA synthesis (fig. S22F), accompanied by a minor change in LINE1 RNA expression (FIG. 27G). All of these findings were different from the changes observed in stem cells, primary tissues, and early development. Moreover, LINE1 RNA knockdown led to a trivial change in chromatin accessibility (FIGS. 27H and 27I), indicating a minor role of LINE1 RNA in chromatin regulation in Mel624 cells.

The inventors then performed ATAC-seq in Mel624 cells and observed an increased overall intensity associated with many more increased ATAC-seq peaks (FIGS. 27J and 27K) upon FTO knockdown. Gained-open regions were enriched in H3K27Ac binding regions in Mel624 cells, with an increased overlap after FTO knockdown (FIG. 27L). Accordingly, the inventors found increased H3K27Ac and reduced H3K27Me3 levels in shFTO Mel624 cells compared to control (FIG. 27M).

Since histone modifications could alternatively be modulated through m⁶A methylation of mRNAs encoding histone modifiers (34), the inventors analyzed the expression and m⁶A level of mRNA encoding modifiers for H3K27Ac and H3K27Me3. Upon FTO knockdown, the inventors found a decreased abundance of the mRNA encoding the H3K27Me3 methyltransferase EZH2, while the transcripts encoding the H3K27Ac acetyltransferase EP300 and the H3K27Me3 demethylase KDM6B were increased (FIG. 27N), accompanied by increased m⁶A levels (FIG. 27O). Previous studies have shown that m⁶A-marked mRNA can be stabilized by IGF2BP (64) or destabilized by YTHDF2 (65), with more targets of FTO belonging to IGF2BP pathways in acute myeloid leukemia (AML) (7-9). Indeed, in FTO-knockdown Mel624 cells, knockdown of YTHDF2 reversed the decreased lifetime of EZH2, whereas triple knockdown of IGF2BP1-3 partially rescued the increased lifetime of EP300 and KDM6B (FIG. 27P).

Lastly, the inventors asked whether m⁶A level and expression of mRNA encoding histone modifiers could also contribute to the altered histone modifications observed in Fto“ ” mESCs. In contrast to the results described above, the inventors did not detect hypermethylated m⁶A peaks on polyadenylated RNA of these transcripts in mESCs (FIG. 8), and only noticed changes in their expression that were either minimal or opposite to what would be expected given the observed chromatin state changes upon Fto KO (FIG. 8). The inventors also checked protein level changes since mRNA m⁶A can impact translation (66). Again, those changes were minor or opposing to the changes expected based on observed chromatin state changes with Fto depletion (FIG. 8). These findings suggest that the effects of FTO on chromatin state in mESCs are unlikely to be due to m⁶A demethylation of mRNAs encoding histone modifiers and support the role of FTO-mediated LINE1 RNA m⁶A demethylation in mESCs.

### FTO Overexpression Improves Reproduction In Mouse, Humans, and Livestock

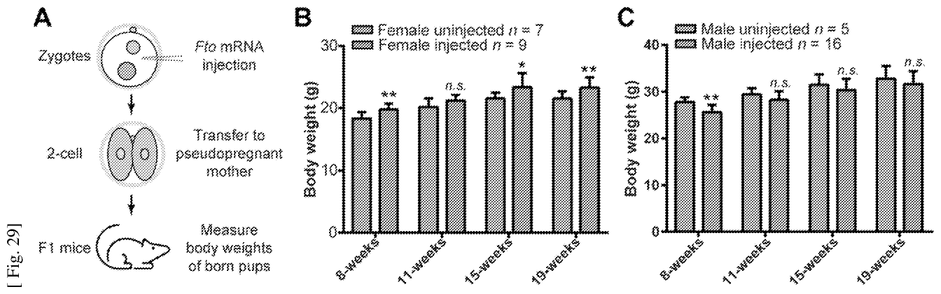

The inventors synthesized Fto mRNA via in vitro transcription, injected 150 ng/μL of Fto mRNA into WT mouse zygotes and transferred the 2-cell embryos into surrogate female mice. The body weights of the pups were measured after maturity. As shown in FIG. 29, the weights of female offspring born from Fto mRNA injected zygotes were significantly bigger than that from uninjected zygotes from 8 weeks to 19 weeks. This is not the case for the male mice born from injected and uninjected zygotes. This data indicates that transient FTO overexpression (OE) in early mammal embryos can partially promote bodyweight growth of FTO transgenic OE mammals. The inventors will perform analogous experiments in other mammals.

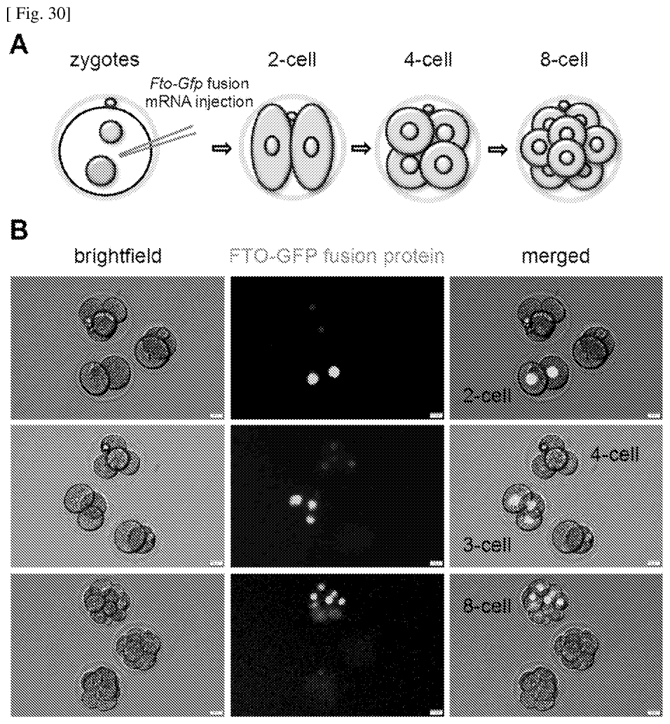

To directly visualize the overexpressed FTO proteins in early embryos, the inventors added a GFP tag to the FTO protein through synthesis of Fto-Gfp fusion mRNA via in vitro transcription. The inventors injected 150 ng/μL of Fto-Gfp fusion mRNA into WT mouse zygotes, cultured them in vitro and monitored the GFP signal under fluorescence microscope. As shown in FIG. 30, there was some variability in the efficiency of FTO OE as indicated by the level of GFP signal among the embryos with or without successful injection or translation of Fto-Gfp fusion mRNA. The injected embryos were stained with anti-FTO antibody, and the signal of anti-FTO overlaps with the GFP signal in the nucleus, indicating the FTO OE was successful (FIG. 30). The GFP positive embryos can be selected for downstream imaging and sequencing analysis and transplant experiments. The uninjected and injected GFP positive and negative embryos can be compared with each other to study the mechanisms of FTO OE. The inventors will use these FTO transient OE mouse embryos to study how overexpressed FTO remodels the m⁶A landscape in early embryos, and how this remodeling contributes to the FTO OE phenotype.

To study how FTO OE affects the transcriptome of early embryos, the inventors will perform single embryo RNA-seq analysis on control and FTO transient OE embryos at different stages. The inventors expect to find a number of differential expressed genes and repeats. To study how the chromatin accessibility and some histone marks are changed by Fto OE, the inventors will perform low-input ATAC-seq and CUT&Tag analysis using control and FTO transient OE embryos at the 8-cell and morula stages.

To find out how FTO OE improves embryo development and reproduction, the inventors focused on the MERVL (mouse ERV with a leucine tRNA primer-binding site) retroelements, which are a subfamily of endogenous retroviruses (ERVs) that particularly active in early embryogenesis. Activation of MERVL retroelements is a hallmark of zygotic genome activation (ZGA) and totipotence of 2-cell mouse embryos. There are 656 full-length copies of MERVL elements in the C57BL/6 mouse genome, and they are among the first sequences to be transcribed at the early 2-cell stage and account for nearly 4% of the mouse transcriptome in 2-cell embryo. Knock down MERVL causes developmental arrest at the 2-cell stage. The level of MERVL RNA is downregulated shortly after the late 2-cell stage, and they are cleared by the morula stage. The molecular basis of rapid MERVL clearance at late pre-implantation stages has not been addressed.

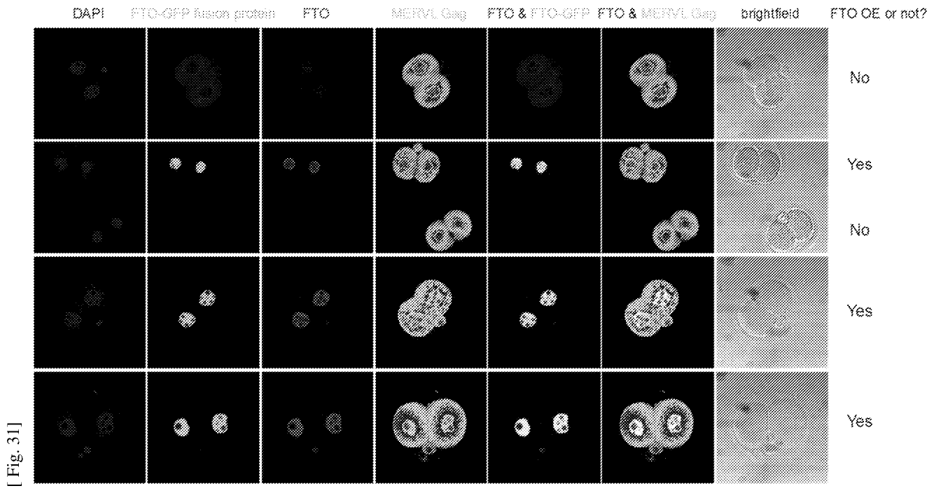

The inventors previously performed m⁶A MeRIP-seq mapping in early mouse embryos and found that MERVL RNAs are heavily marked with m⁶A. Knock down m⁶A writers Mettl3/14/16 or treatment with METTL3 inhibitor STM2457 increased MERVL RNA level at the morula stage due to failure of m⁶A-mediated RNA decay. FTO OE may erase the m⁶A on MERVL RNAs and prevent them from RNA decay. Increased level of and longer persist MERVL expression may promote embryo development and increase the weights of embryos and pups before and after birth. The inventors stained the Fto-Gfp fusion mRNA injected embryos at 2-cell stage with anti-FTO and anti-MERVL Gag antibodies. The 2-cell embryos with FTO OE have stronger MERVL Gag signal than the 2-cell embryos without FTO OE, especially in the nucleus (FIG. 31). The inventors will look at different stages including the morula. In addition to MERVL, there are other retrotransposon RNAs that could also serve as substrates of overexpressed FTO, including homologs in other species. Additionally, the inventors have previously shown that FTO overexpression could induce tRNA fragment formation, which may also affect embryo development. All mechanisms possibly affect embryo development, implantation, and post-birth body weight.

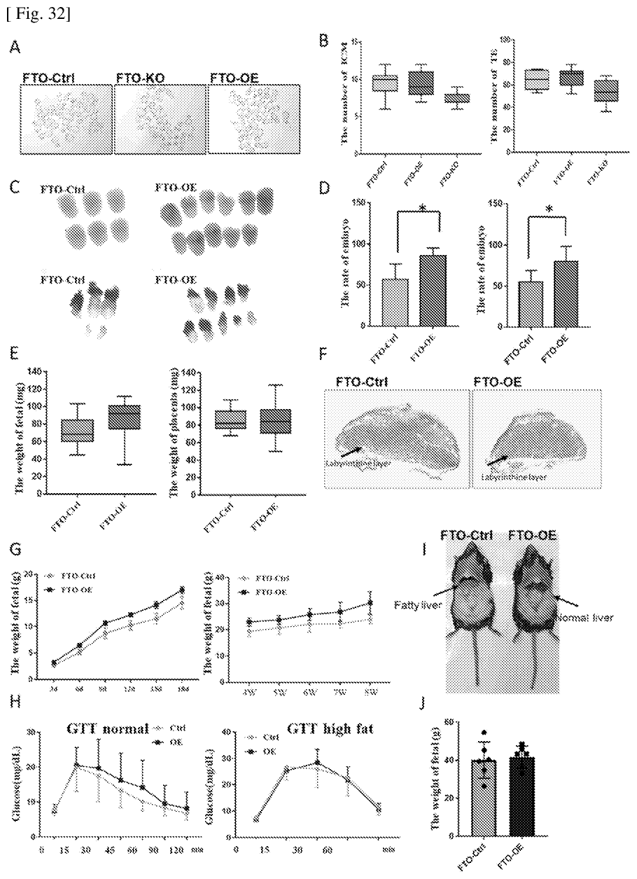

Assisted reproductive technology (ART) has been widely applied in the treatment of human infertility and for animal breeding. However in vitro fertilization (IVF), which is the commonly used in ART procedure, have been shown to cause short-term and long-term health problems, including pregnancy loss, placental abnormalities, low birth weight, and metabolic diseases in the offspring. In previous studies in mice, the inventors found IVF disrupted histone modification and expression of extraembryonic tissue-specific gene during implantation, which further resulted in reduced cell numbers of ICM and TE in E3.5 blastocyst, thus altering embryonic weight and placental development in E13.5-E18.5 (https://pubmed.ncbi.nlm.nih.gov/35508139/). The inventors speculated that FTO transient OE could promote development of IVF embryo and improve ART processes in mammals. The inventors injected 150 ng/μL of Fto mRNA (FTO-OE) into mouse zygotes which were fertilized in vitro, and injections of water (FTO-Ctrl) and sgRNA+Case9 mRNA (FTO-KO) were performed as controls. The inventors found that the loss of embryonic expression of FTO when using the CRISPR system (FTO-KO) inhibited dilatation and hatching from the zona pellucida at the late blastocyst stage (E4.5), which are required for implantation and intrauterine growth, and the cell numbers of ICM and TE were reduced significantly (FIGS. 32A and 32B).

In contrast, FTO-OE embryos showed higher quality than FTO-Ctrl embryos, albeit modestly (FIGS. 32A and 32B). The inventors then transplanted FTO-Ctrl and FTO-OE embryos into surrogate female mouse at 2-cell stage. Fifteen 2-cell embryos of FTO-Ctrl and FTO-OE embryos were transplanted into the tubal of one pseudopregnant female mice with more than three acceptor mice for each batch. The number of deciduas and embryos was counted and the ratio of embryo/decidua was calculated at E7.5. The inventors found that FTO transient OE in zygotes increased the implantation ratio from 55% in FTO-Ctrl embryos to 85% in FTO-OE embryos at E7.5 (FIGS. 32C and 32D), and similar increases were observed with different genetic background (FIG. 32D, BDF1 cross BDF1 for left panel and C57 cross DBA2 for right panel). The increases may be observed in other species, including livestock species such as pigs and cows. The inventors further measured the weights of FTO-Ctrl and FTO-OE embryos and placentas at E12.5. The inventors found that FTO transient OE in zygotes increased the body weights of embryos by ˜10-20% at E12.5 (FIG. 32E). FTO transient OE also facilitated the development of labyrinthine layer in the placenta (FIG. 32F). The inventors traced the weight of the FTO-Ctrl and FTO-OE pups from birth to maturation. The weight gain of FTO-OE pups were faster in the first 8 weeks. Moreover, the blood glucose of FTO-OE mice appeared normal at 8 week with normal or high fat diet at week 8-9 (FIG. 4H). Importantly, the inventors found that after 3 months of high-fat diet, the final body weight of FTO-OE mice was similar to FTO-Ctrl but they were less susceptible to severe fatty liver disease (FIGS. 41 and 4J). This shows that transient expression of FTO can help reduce the risk of metabolic diseases in IVF offspring.

In summary, shown herein are simple methods to increase the weight and number of offspring of animals, including by overexpressing FTO in fertilized cells, including injecting the Fto mRNA into zygotes. FTO OE may erase the m⁶A on MERVL RNAs and prevent them from RNA decay, thus promote embryo development and increase the weights of embryos and pups before and after birth.

These findings in FTO transient OE mouse embryos together with previous studies in FTO OE rice and potato increasing yield supports FTO OE in livestock can improve reproduction and yield in animal husbandry and also that FTO OE in human can improve IVF efficiency and health.

Livestock. As used herein “livestock” refer to animals, including mammals, raised for commercial purposes including consumption, labor, and/or products (including milk, fur, wool, skin, hides, or other commercial products). Livestock includes, but is not limited to cattle, swine, sheep, goats, horses, and mules.

Companion Animals. As used herein “companion animals” refer to domesticated animals that are commonly kept in a household, and may be used as service or support animals. Companion animals includes, but is not limited to, horses, dogs, cats, rabbits, mice, and rats.

Research and Biomedical Product Animals. As used herein “research animals,” which may be interchangeable with “biomedical product animals” refers to animals raised for research, including as model animals, or biomedical products. Research animals include, but are not limited to, mice, rats, pigs, dogs, and primates.

Endangered Animals. As used herein “endangered animals” refer to animals typically found in the wild that are at risk of becoming extinct. Endangered animals can be animals for which conservation efforts include in vitro fertilization to increase the population of the endangered animal. Endangered animals include, but are not limited to, pandas, bison, polar bear, lions, and tigers.

Livestock applications include the use of FTO overexpression in livestock reproduction for increased body weight, enhanced prolificacy, reproductive performance, growth rate, increased use of feed, improved milk production and/or composition, modification of hair or fiber, climate resistance, consumption product improvement, cloning efficacy, improved breeding of transgenic animals. Companion animal applications include the use of FTO overexpression to improve reproduction for increased body weight, enhanced prolificacy, reproductive performance, efficient feed use, disease resistance, and climate resistance. Additionally, the production of designer companion animals (e.g. cats without allergens), companion animal cloning, or the improved fitness of a specific breed are also applications. FTO overexpression may be used for improved reproduction for increased body weight, enhanced prolificacy, reproductive performance, efficient feed use, disease modeling, cloning, xenotransplantation products. Additionally, engineered farm species that express medically important proteins in their milk would be another use of mammals for biomedical products. Endangered wildlife applications include the use of FTO overexpression to improve reproduction for increased body weight, enhanced prolificacy, reproductive performance, growth rate, climate resistance and disease resistance, and for propagation of a threatened or endangered species.

### Materials and Methods

### General Methods

Fto^{-/-} and control WT mESCs were derived from the inner cell mass (ICM) of E3.5 blastocysts. m⁶A-IP was performed for non-ribosomal RNA isolated from the chromatin-associated fraction or from whole cell, as indicated, using the EpiMark N⁶-Methyladenosine Enrichment Kit (NEB). All the RNA sequencing libraries were prepared using SMARTer Stranded Total RNA-Seq Kit v2-Pico Input Mammalian (TaKaRa). For most samples, libraries were sequenced on an Illumina NovaSeq 6000 in a 100-bp paired-end mode. For RNA sequencing data, Trimmomatic trimmed reads were aligned to the mm10 reference genome using HISAT2; read counts were calculated by featureCounts and differential expression was analyzed using DESeq2. For the generation of pre-implantation embryos, MII oocytes were subjected to intracytoplasmic sperm injection (ICSI) and embryo culture. Detailed materials and methods are available in the supplementary materials.

### Animals and Tissues

FTO heterozygous mice were gifts from the laboratories of Dr. Pumin Zhang and Xuekun Li as described previously (37). Fto^{-/-} mice were generated by intercrossing FTO heterozygous mice. WT, paternal, maternal, and double depletion of Fto embryos (referred as Fto^{P+/M+}, Fto^{P+/M-}, Fto^{P-/M+}, and Fto^{P-/M-}, respectively) were obtained by performing Intracytoplasmic sperm injection (ICSI) using WT MII and Fto^{-/-} MII with WT sperm and Fto^{-/-} sperm, respectively.

For H&E staining, ovaries of female mice (4-week-old or 7-week-old) were harvested and immediately fixed in 4% paraformaldehyde overnight at 4° C. Then, the ovaries were washed with current water for 4 h followed by dehydration with an increasing concentration of ethanol, vitrification by dimethylbenzene, and embedding in paraffin wax. The paraffin-embedded tissues were then cut to a thickness of 5 μm, and then stained with H&E after deparaffinization and rehydration. The tissues were imaged using Olympus SLIDEVIEW VS200.

Germinal vesicle (GV) oocytes were obtained from ovaries of female mice (3-4-week-old), 48 h after intraperitoneal injection of pregnant mare serum gonadotropin (PMSG) (San-Sheng Pharmaceutical). To get MII oocytes and pre-implantation embryos, female mice (6-8-week-old) were superovulated by injection with 7 IU each of PMSG, followed by injection with 5 IU of human chorionic gonadotropin (hCG) (San-Sheng Pharmaceutical) 48 h later. MII oocytes were collected from the oviducts of unmated female mice.

For ICSI and in vitro embryo culture, sperm was isolated from the caudal epididymis of adult male mice with clippers. To inject fresh spermatozoa, approximately 1 μL of the incubated sperm suspension was mixed with a drop of HEPES-buffered Chatot-Ziomek-Bavister (HCZB) containing 10% (w/v) polyvinylpyrrolidone (PVP; Irvine Scientific, Santa Ana, CA, USA). The sperm head was separated from the tail by the application of several Piezo pulses, and the head was then injected into the oocyte. HCZB medium was used for gamete handling and ICSI in air. The fertilized embryos were cultured in G1 medium (Vitrolife) at 37° C. under 5% CO2 in air. The embryos at morula stage were then collected for RNA extraction and DNase I-TUNEL assay as described in the below sections. The embryos at E3.5 blastocyst stage were transferred into the uteri of pseudo-pregnant ICR female mice, and the development rate was recorded with each group at both E3.5 early blastocyst and E4.5 blastocyst stages.

To obtain post-implantation embryos, female mice at 7.5 days post-coitum (d.p.c.) were executed and the uteri were dissected and transferred to a petri dish with PBS. Next, each decidua was carefully freed from the uterine muscle layers using properly sharpened forceps. Decidua numbers were recorded and the implantation rate was calculated as decidua numbers divided by implanted embryo numbers in each group. Then 7.5 d.p.c. embryos were carefully separated from deciduas. Reichart's membrane and the ectoplacental cone were also removed from the embryos. E7.5 embryo rate was defined as the number of E7.5 embryos divided by the number of implanted embryos in each group.

Fto^{-/-} mouse brain tissues and the WT controls were obtained from the laboratories of Dr. Xuekun Li. For brain section preparation, mice were deeply anesthetized with chloral hydrate (50 mg/kg, i.p.) and transcardially perfused with cold phosphate-buffered saline (PBS) followed by perfusion of 4% paraformaldehyde (PFA). The mouse brain sample was carefully taken out and immediately soaked in 4% PFA overnight at 4° C. After 24 h, the brain samples were completely dehydrated with 30% sucrose solution at 4° C. The brain samples were embedded in Optimal Cutting Compound (O.C.T., Thermo Scientific) and sliced into 20 μm slices. For qPCR analysis, the brains of the mice were taken out and rinsed in PBS. Then the cerebellum tissue and hippocampus tissue were separated on ice, washed with PBS three times, and the tissues were placed in TRIzol reagent (Thermo Scientific) for total RNA isolation.

WT mice (6-8 weeks old) used for RT-qPCR in tissue correlation analysis and related experiments were approved by the University of Chicago Institutional Animal Care and Use Committee.

Construction of Chimeric Mice with Fluorescent Protein-Labeled ESC Lines

HEK293T cells were infected by lentivirus with pSicoR-Wf1a-mCherry plasmid and lentiviral packaging plasmids psPAX2 and Pmd2.G using VigoFect reagent (Vigorous Biotechnology). After 48 h, supernatant medium was collected with released virus and concentrated with 10% PEG 8000 (Sigma) for approximately 12 h at 4° C. The mixture was centrifuged for 20 min at 4° C. to pellet viral and resuspended with ESC medium. Then, ESCs were infected with the concentrate for 10 h and transferred to a petri dish with feeder cells. After 72 h, the RFP+ ESC clones were picked using a glass needle to obtain RFP+ monoclonal cells.

To construct chimeric mice, after treated with hormone, ICR female mice were mated with male mice to obtain E3.5 blastocysts. Approximately 15 RFP+ ESCs were microinjected into each E3.5 blastocyst and then implanted into surrogate mice. After 12.5 days, surrogate mice were dissected to obtain fetuses for further investigations. The extent of chimerism in each fetus was determined by the percentage of RFP+ cells using FACS analysis on the skin tissues.

### Meiotic Spindle Staining of Mouse Oocytes

MII oocytes were collected and fixed in 4% paraformaldehyde in PBS for 30 min at room temperature. They were washed three times with 1% FBS in PBS, and were transferred to a membrane permeabilization solution with 0.5% Triton X-100 in PBS for 30 min at room temperature and then blocked with 5% BSA in PBS for 30 min. After three rinses, oocytes were stained with anti-α-tubulin overnight at 4° C. Next, oocytes were washed three times with 1% FBS in PBS and then incubated with secondary antibody for 2 h at room temperature. After washing with 1% FBS in PBS three times, chromosomes were labeled using Hoechst 33342 (KeyGen BioTECH, KGA212-10) for 3 min and mounted in quenching agent drops on a glass slide for confocal imaging with a ZEISS LSM 800 microscope.

### Cell Culture

shNC and shFTO Mel624 cell lines were obtained from the laboratory of Dr. Yu-Ying He. Mel624 cells were maintained in DMEM (Gibco) with 10% fetal bovine serum (FBS, Gibco), 0.5 μg/mL puromycin (Gibco) and 1× Pen/Strep (Gibco) at 37° C. with 5% CO₂. WT and Fto^{-/-} mouse embryonic stem cells (mESCs) were derived from the inner cell mass (ICM) of E3.5 blastocyst. mESCs were maintained in DMEM (Invitrogen) supplemented with 15% stem cell-qualified FBS (GeminiBio), 1% nucleosides (100×) (Millipore), 1 mM L-glutamine (Gibco), 1% nonessential amino acid (Gibco), 0.1 mM 2-mercaptoethanol (Sigma), 1,000 U/ml LIF (Millipore), 3 μM CHIR99021 (Stemcell) and 1 μM PD0325901 (Stemcell) at 37° C. and 5% CO2. Half of the medium was replaced every day. WT and Fto^{-/-} mouse adult neural stem cells (aNSCs) were derived as described previously (38), cultured in DMEM/F12 medium supplemented with 1% N2 (Invitrogen) and 2% B27 (Invitrogen), 20 ng/ml basic fibroblast growth factor (bFGF; R&D Systems), and 10 ng/ml epidermal growth factor (EGF; Peprotech) at 37° C. and 5% CO₂. Half of the medium was replaced every other day.

### Alkaline Phosphatase (AP) Staining, Cell Cycle Analysis, and Proliferation and Differentiation Assays

AP staining was performed using the BCIP/NBT Alkaline Phosphatase Color Development Kit (Beyotime) following the manufacturer's protocol. In brief, mESCs were washed twice by DPBS, fixed with 4% paraformaldehyde for 10 min at room temperature, and stained with the BCIP/NBT Solution.

For cell cycle analysis, cells at 80% confluency were harvested by trypsin and fixed by 70% ethanol at 4° C. for 30 min. Then samples were washed with PBS twice and stained in PBS solution with 20 μg/ml RNase A and 40 μg/ml Propidium Iodide (PI) at room temperature for at least 30 min. The stained samples were directly sent for flow cytometry on the BD Fortessa (BD), and data were analyzed by Flowjo.

The cell proliferation was measured by assaying cells at various time points using the CellTiter 96® Aqueous One Solution Cell Proliferation Assay (Promega) following the manufacturer's protocols. 5000 cells were seeded per well in a 96-well plate at day 0.

For EB differentiation, mESCs were trypsinized and a total of 10⁵ cells per well were cultured in ultra-low cluster plates (Costar) in DMEM (Gibco) supplemented with 15% FBS (Gibco), 1 mM L-glutamine (Gibco), 0.1 mM mercaptoethanol (Sigma), 1% nonessential amino acid (Gibco). EBs were collected and counted every 48 h after seeding until six days. Total RNA of EBs was extracted and analyzed for LINE1 RNA and the marker genes using RT-qPCR.

For aNSC differentiation, aNSCs were passaged 1:1 to a well-coated cell culture plate and grown overnight. The composition of the differentiation medium was as follows: 1 μM retinoic acid and 5 μM forskolin are used to replace EGF and FGF-2 in the growth medium, and the rest of the composition remaining unchanged.

### DNase I-TUNEL Assay

For cell line samples, mESCs, aNSCs, or Mel624 cells were resuspended and transferred to a Nunc Lab-Tek II Chamber Slide (8-well, Thermo Scientific) 16 h prior to treatment. The DNase I-TUNEL assay was performed using the DeadEnd Fluorometric TUNEL System (Promega) following the manufacturer's protocols. Three independent experiments were performed. Cells were treated with 1 U/mL of DNase I (Thermo Scientific) for 5 min at 37° C. before rTdT labeling. Cell nucleus was counterstained with DAPI (Sigma). ProLong Diamond Antifade Mountant (Invitrogen) was added before fluorescence imaging capture. More than five images were captured in each independent experiment. Images were captured with Olympus FV1000 microscope. Flow cytometry was performed on a BD Fortessa (BD), and data were analyzed using Flowjo (Treestar).

GV oocytes and morula samples were collected and washed twice with PBS. In vivo permeabilization was performed with 0.5% Triton X-100 in extraction buffer (50 mM NaCl, 3 mM MgCl2, 0.5% Triton X-100 and 300 mM sucrose in 25 mM HEPES, pH 7.4) for 5 min on ice. Samples were washed twice in extraction buffer without Triton X-100 and then moved into 1 U/ml DNase I in the same buffer for 5 min at 37° C. Then, 2% PFA was applied for 10 min at room temperature. The DNase I-TUNEL assay was performed using DeadEnd Fluorometric TUNEL System (Promega) following the manufacturer's protocols. All samples were stained with DAPI (1 μg/ml) for 20 min. Lastly, samples were manipulated using a mouse pipette and were reacted in a 96-well U-type plate and finally transferred to PBS drops covered by paraffin oil on a glass-bottom cell culture dish (NEST) for imaging with a ZEISS LSM 880 microscope.

For brain tissues, TUNEL assays were performed using In Situ Cell Death Detection Kit (TMR red; Roche 12156792910) following the manufacturer's instructions. Slides were placed in a plastic jar containing 200 ml 0.1 M Citrate buffer, pH 6.0. 750 W (high) microwave irradiation was applied for 1 min. The slides were cooled rapidly by immediately adding 80 ml double distilled water (20 to 25° C.). Then the slides were transferred into PBS (20 to 25° C.). The slides were immersed for 30 min at 15 to 25° C. in Tris-HCl, 0.1 M pH 7.5, containing 3% BSA and 20% normal bovine serum. They were then treated with a TUNEL reaction mixture containing 90% terminal deoxynucleotidyl transferase (TdT) buffer, 5% dUTP-biotin and in 5% TdT in a humid dark box at 3° C. for 1 h. After being rinsed three times in PBS, sections were counterstained with DAPI. Images were captured with an Olympus FV3000 microscope.

For all the image samples mentioned above, the TUNEL signal intensity in each cell nucleus was quantified and normalized to DAPI using Fiji (ImageJ) software based on >10 images in three independent experiments except for oocyte and embryo samples (n is indicated in the figure legend). The inventors considered each nucleus as one object and did unpaired two-tailed t-test for comparison between WT and Fto KO samples.

### Nascent RNA Labeling Assay

mESCs, aNSCs, or Mel624 cells were resuspended and transferred to Nunc Lab-Tek II Chamber Slide (8-well, Thermo Scientific) 16 h prior to treatment. The nascent RNA synthesis assay was performed using Click-It RNA Imaging Kits (Invitrogen) following the manufacturer's protocols. Cell nucleus was stained with DAPI (Sigma). ProLong Diamond Antifade Mountant (Invitrogen) was added before fluorescence imaging capture. Images were captured with Olympus FV1000 microscope and intensity of EU signal was quantified using ImageJ (Fiji) software based on >10 images in three independent experiments.

LINE1 FISH and Co-Staining with FTO Immunofluorescence

LINE1 RNA FISH was performed following the manufacturer's standard protocol (Biosearch Technologies). In brief, cells were cultured on 10 mm #1.5 cover glass. Fixation was performed in 4% paraformaldehyede (PFA) for 10 min, followed by permeabilization for 20 min in 0.5% Triton dissolved in PBS. Samples were washed by Stellaris® RNA FISH Wash Buffer A with 10% Formamide, and blocked by 1 μg/μl yeast tRNA (Thermo Scientific) at 37° C. for 2 h. Probes targeting LINE1 RNA were diluted in Stellaris® RNA FISH Hybridization Buffer with 10% Formamide at 1:100 dilution to final concentration of 12.5 nM. Hybridization was performed in a humid environment at 37° C. for 16 h. Samples were washed by RNA FISH Wash Buffer A and stained with DAPI. The intensity of fluorescence signals was quantified and normalized to DAPI using Fiji (ImageJ), where the inventors considered each nucleus as one object and did an unpaired two-tailed t-test for comparison between WT and Fto KO samples.

FISH co-staining with FTO immunofluorescence was performed following the manufacturer's standard protocol (Biosearch Technologies). The inventors used 70% ethanol to precipitate the sample at 4° C. for 20 min before probe hybridization as an optimized step for co-staining. After the hybridization of FISH probes, samples were equilibrated by PBS and blocked by blocking buffer (2% BSA, 0.05% Triton, 0.2 U/μl SUPERase·In™ RNase Inhibitor in PBS) for 1 h. FTO antibody (Abcam) at 1:100 dilution in blocking buffer was incubated for 1 h at room temperature. Samples were washed by blocking buffer and stained by Alexa 488 conjugated 2^{nd} antibody (Invitrogen) for 1 h. Then samples were stained by DAPI and fixed again by 4% PFA for 10 min. The intensity of FISH signals was quantified and normalized to DAPI using Fiji (ImageJ), where the inventors considered each nucleus as one object and did unpaired two-tailed t-test for comparison between WT and Fto KO samples. The colocalization of two signals were measured by the Coloc2 plugin in ImageJ, and Pearson correlation coefficient of 12 images was statistically tested by one-sample t-test. For all the experiments mentioned in this section, Images were captured by Leica SP8 Laser Scanning confocal microscope. Three independent experiments were performed and more than five images were captured in each experiment.

### LINE1 Antisense Oligo (ASO) Knockdown

A published morpholino ASO targeting mouse LINE1 was used for LINE1 knockdown, with its reverse complement (RC) as a control (Data S2) (27). Morpholino ASO targeting ORF2 of human LINE1 was designed with software available at GENE Tools LLC. Morpholino ASO was labeled with FITC for the detection of nucleofection. Nucleofection of mESCs and Mel624 cells was performed on an Amaxa™ 4D-Nucleofector (Lonza) using a P3 Primary Cell 4D-Nucleofector™ X Kit (Lonza) and a SF Cell Line 4D-Nucleofector X Kit (Lonza), respectively, following the manufacturer's standard protocol. 5×10⁶ cells were used for nucleofection with 5 nmol of ASO in a 100 μl system.

dCas 13b Plasmid Transfection

mESCs at 80% confluency were split at a 1:6 ratio 16 h prior to plasmid transfection. Transfection was achieved using Lipofectamine 3000 Transfection Reagent (Invitrogen) for dCas13b plasmids following the manufacturer's protocols and a published protocol (39). The mass ratio of dCas13b plasmids and gRNA plasmids was maintained at 3:1. The transfected cells were cultured for 2 days before further analysis. LINE1 gRNA was designed to target the top LINE1 RNA subfamilies responding to Fto KO (Data S2).

For the application in early embryos, dCas13b-FTO mRNA was in vitro transcribed from the DNA template with a T7 promoter using the mMESSAGE mMACHINE T7 Ultra Transcription Kit (ThermoFisher Scientific) following the manufacturer's protocol. dCas13b-FTO mRNA (200 ng/μl) and LINE1 sgRNA (50 ng/μl) were mixed and injected into the MII cytoplasm by a Piezo impact-driven micromanipulator, and ICSI was performed using FTO KO sperm in HCZB medium as described above. The fertilized embryos were cultured in G1 plus medium at 37° C. under 5% CO2 in air and embryos at morula stage were collected for RNA extraction and DNase I-TUNEL assay.

### Cell Fractionation

Fractionation of mESCs or Mel624 cells was performed following a published protocol (40) with the concentration of NP40 (Sigma) optimized for each cell line. In brief, 5×10⁶ to 10⁷ cells were harvested and washed with 1 mL cold PBS/1 mM EDTA buffer, then centrifuged at 4° C. at 500 g to collect the cell pellet. 200 μL ice-cold lysis buffer (10 mM Tris-HCl, pH=7.5, 0.05% NP40, 150 mM NaCl) was added to the cell pellet and incubated on ice for 5 min, then the cell lysate was gently pipetted up over 2.5 volumes of chilled sucrose cushion (24% RNase-free sucrose in lysis buffer) and centrifuged at 4° C. at 15,000 g for 10 min. All of the supernatant was collected as the cytoplasmic fraction and the nuclei pellet was washed once by gently adding 200 μL ice-cold PBS/1 mM EDTA to the nuclei pellet without dislodging the pellet. The nuclei pellet was resuspended in 200 μL prechilled glycerol buffer (20 mM Tris-HCl, pH=7.9, 75 mM NaCl, 0.5 mM EDTA, 0.85 mM DTT, 0.125 mM PMSF, 50% glycerol) with gentle flicking of the tube. Then an equal volume of cold nuclei lysis buffer (10 mM HEPES, pH=7.6, 1 mM DTT, 7.5 mM MgCl2, 0.2 mM EDTA, 0.3 M NaCl, 1 M UREA, 1% NP-40) was added, followed by vigorously vortexing for 5 seconds twice. The nuclei pellet mixtures were incubated for 2 min on ice, then centrifuged at 4° C. at 15,000 g for 2 min. The supernatant was collected as the soluble nuclear fraction (nucleoplasm). The pellet was gently rinsed with cold PBS/1 mM EDTA without dislodging and then collected as the chromosome-associated fraction.

### Western Blot

Protein samples were prepared from respective cells by lysis in RIPA buffer (Invitrogen) containing 1× protease inhibitor cocktail (Roche). Protein concentration was measured by NanoDrop 8000 Spectrophotometer (Thermo Scientific). Lysates were boiled at 95° C. with 4× loading buffer (Biorad) for 5 min. Denatured protein was loaded into 4-12% NuPAGE Bis-Tris gel and transferred to PVDF membranes (Life Technologies). Membranes were blocked in PBST with 5% BSA for 30 min at RT, incubated in a diluted primary antibody solution at 4° C. overnight, washed, and incubated in a dilution of secondary antibody conjugated to HRP for 1 h at room temperature. Protein bands were detected using SuperSignal West Dura Extended Duration Substrate kit (Thermo) on FluroChem R (Proteinsimple). The intensity of each band was measured by ImageJ, and quantified based on the area of the intensity plot (target proteins are normalized to loading control indicated in the figure legends).

RNA Purification from Cell Samples

Total RNA from whole-cell or chromatin-associated fractions was purified with TRIzol reagents (Thermo Scientific) and the rRNA was removed using a RiboMinus Eukaryote kit (Thermo Scientific). Total RNA samples used for RT-qPCR were treated with an additional on-column DNase digestion step. Polyadenylated RNA was purified from total RNA with two rounds of polyA-tail purification using the Dynabeads® mRNA DIRECT™ kit (Thermo Scientific). RNA concentration was measured by NanoDrop 8000 Spectrophotometer (Thermo Scientific) or a Qubit Fluorometer (Thermo Scientific).

### Quantitative Analysis of m⁶A Levels Via UHPLC-MS/MS

50 ng non-ribosomal RNA from cell fractionation or polyadenylated RNA was digested by nuclease P1 (Sigma) in 20 μl of buffer containing 25 mM NaCl and 2.5 mM ZnCl₂ for 1 h at 42° C. Subsequently, 1 unit of FastAP (Thermo Scientific) in 10× FastAP buffer was added and the sample was incubated for 4 h at 37° C. The samples were then filtered (0.22 μm, Millipore) and injected into a C18 reverse-phase column coupled online to Agilent 6460 LC-MS/MS spectrometer in positive electrospray ionization mode. The nucleosides were quantified using retention time and the nucleoside to base ion mass transitions (268 to 136 for A; 282 to 150 for m⁶A). Quantification was performed by comparing with the standard curve obtained from pure nucleoside standards running with the same batch of samples. The m⁶A level was calculated as the ratio of m⁶A to A.

### MeRIP-qPCR and RT-qPCR for RNA Samples

Total RNA from whole-cell or the chromatin-associated fraction was reverse transcribed using Maxima™ H Minus cDNA Synthesis Master Mix (Thermo Scientific). RT-qPCR was performed using FastStart Essential DNA Green Master (Roche). Relative expression level of each gene was normalized to the reference gene Actb or Hprt for cell samples or H2afz for tissue and embryo samples. m⁶A-IP without fragmentation of total RNA after ribosomal RNA depletion from whole-cell or the chromatin-associated fraction was performed using the EpiMark N⁶-Methyladenosine Enrichment Kit (NEB) following the manufacturer's protocols. m⁶A and non-m⁶A spike-ins from this kit were used as the normalization controls for m⁶A level analysis in MeRIP-RT-qPCR. Primers used for qPCR are listed in Data S2. Relative changes in expression were calculated using the ΔΔCt method.

Cross-Linking Immunoprecipitation and qPCR (CLIP-qPCR)

CLIP experiments were performed following a published paper (16) with slight modifications. In brief, antibodies targeting the protein of interest were pre-conjugated to 1:1 Protein A/G Magnetic Beads (Thermo Scientific). Cells in a 10 cm dish at 80% confluency were cross-linked twice with UV irradiation (254 nm, 150 mJ/cm²) using Stratalinker on ice. Cells were then harvested and lysed and subjected to immunoprecipitation. For region-specific CLIP, RNase T1 was added before immunoprecipitation to 0.1 U/μl and incubated at 22° C. for 10 min. Immunoprecipitated DNA was then purified with TRIzol reagents (Thermo Scientific) and analyzed by qPCR.

Chromatin Isolation by RNA Purification and qPCR (ChIRP-qPCR)

ChIRP was performed as previously described (27) with some modifications. DNA probes were biotinylated through terminal transferase (NEB) with Bio-N6-ddATP (ENZO) as a substrate. mESCs were crosslinked with 3% formaldehyde for 30 min at room temperature, then quenched with 250 mM glycine at room temperature for 5 min. Cells were centrifuged and the pellet was washed with PBS three times. Crosslinked cells were resuspended with cell lysis buffer (CLB, 50 mM Tris-HCl, pH=7.0, 10 mM EDTA, 1% SDS, 0.5 mM DTT, and RNase inhibitors) and sonicated by Bioruptor pico. Chromatin was diluted in two times the volume of hybridization buffer (750 mM NaCl, 1% SDS, 50 mM Tris-HCl, pH=7.0, 1 mM EDTA, 15% formamide, 0.5 mM DTT, and RNase inhibitors). Biotinylated probes (10 pmol per 3 million cells) were added to diluted chromatin and incubated at 37° C. for 6 h with rotation. Streptavidin-magnetic Cl beads were washed three times in nuclear lysis buffer, blocked with 500 ng/μl yeast total RNA, and 1 mg/ml BSA for 1 h at room temperature. Precoated beads were then added and incubated for another 2 h at 37° C. The bound fractions were washed five times with wash buffer (2×SSC, 0.5% SDS, add DTT and PMSF fresh). DNA was eluted twice with a cocktail of 100 μg/ml RNase A and 0.1 U/mL RNase H at 37° C. with end-to-end rotation and eluent from both steps was combined. Reverse crosslinking was carried out at 65° C. overnight and DNA was then extracted with an equal volume of phenol: chloroform: isoamyl alcohol and precipitated with ethanol at −80° C. overnight. Eluted DNA was subjected to qPCR analysis.

DNA-RNA Immunoprecipitation and qPCR (DRIP-qPCR)

DRIP experiments were performed following a published protocol (41). In brief, cells were cultured to ˜80% confluency before harvest and DNA extraction. Enzyme-digested genomic DNA was immunoprecipitated with S9.6 antibody (MilliporeSigma, MABE1095). Immunoprecipitated DNA was then purified with proteinase K treatment and recovered by ethanol precipitation with glycogen (ThermoFisher, 10814010) and subjected to qPCR analysis.

Nuclear RNA Lifetime Measured by qPCR

Six 6-cm plates of WT and Fto^{-/-} mESCs were seeded and controlled to afford the same number of cells, respectively. After 48 h, actinomycin D was added to 5 μg/mL at 6 h, 3 h, and 0 h before trypsinization collection. The entire nuclear fraction was separated as mentioned in the cell fractionation section and total RNA was purified by TRIzol reagents (Thermo Scientific). 1:1000 diluted m⁶A and non-m⁶A spike-ins from EpiMark N⁶-Methyladenosine Enrichment Kit (NEB #E1610S) were added proportional to total nuclear RNA to each sample, and RNA quantities were then determined by RT-qPCR. Half lifetime (t_{1/2}) was calculated using one phase decay model in GraphPad Prism.

Nascent RNA Synthesis Measured by qPCR

Eight 6-cm plates of WT and Fto^{-/-} mESCs were seeded and controlled to afford the same number of cells, respectively. After 48 h, 5-Ethynyl Uridine (EU) was added to 0.5 mM at 60 min, 40 min, 20 min, and 10 min before trypsinization collection. Total RNA was purified by TRIzol reagents (Thermo Scientific) and nascent RNA was captured using the Click-iT Nascent RNA Capture Kit (Invitrogen) following the manufacturer's protocols. 1:1000 diluted m⁶A and non-meA spike-in from the EpiMark N⁶-Methyladenosine Enrichment Kit (NEB #E1610S) were added proportional to total RNA to each sample, and RNA quantities were then determined by RT-qPCR. RNA amount and EU labeling time were fitted to a linear equation in GraphPad Prism, and the slope was used to estimate the transcription rate of RNA.

### Qpcr Primer Design and Validation for Specific LINE1 Subfamilies and LINE1 RNA Loci

qPCR primers targeting L1Md_Tf and L1Md_A were obtained based on previously published sequences (27). The inventors further validated the specificity of these primers to indicated LINE1 subfamilies through NCBI Primer-BLAST (https://www.ncbi.nlm.nih.gov/tools/primer-blast/). In brief, the inventors submitted the defined primers to Primer-BLAST using consensus sequences of all LINE subfamily obtained from the Dfam database (https://dfam.org/home) as the custom reference database, with the repeat filter set to “none”. The inventors confirmed that no PCR product on other LINE1 subfamilies (with size 70-300 and no primer mismatch) besides the targeted LINE1 subfamily of interest could be obtained from the published primer pairs. The primer pairs were also validated to have no nonspecific targets using Refseq as a reference database.

To design qPCR primers targeting a certain specific LINE1 subfamily, the consensus sequence of the targeted LINE1 RNA subfamily was considered as a PCR template, with a PCR product size range from 70 to 300 bp. The primers were designed using consensus sequences of all LINE subfamilies obtained from the Dfam database (https://dfam.org/home) as the custom reference database, with the repeat filter set to “none”. All other parameters were the default parameters of Primer-BLAST. The designed primers were validated as described above. To design qPCR primers targeting a specific intragenic LINE1 locus, the sequence of the specific targeted LINE1 locus was considered as a PCR template, with a PCR product size range from 70 to 300 bp. The primers were designed using the mouse genomic sequence as a reference database, with the repeat filter set to “none”. All other parameters were default parameters of Primer-BLAST. Thus, the inventors confirmed that each primer pair generates no PCR product on other targets with a size between 70 and 300 bp and no primer mismatch.

The inventors recognize that for certain LINE1 subfamilies, the criteria of nonspecific targeting for typical primer design (more than 5 mismatches on each qPCR primer) may not be fully satisfied and may generate PCR products on LINE1 subfamilies other than targeted one. The inventors validated the specificity of the primers targeting specific intragenic LINE1 RNA loci used herein. However, for certain specific intragenic LINE1 RNA loci, the primer designed by Primer-BLAST may have multiple targets along the genome due to the repetitive nature of LINE1 RNA.

### RNA-Seq for Nuclear RNA Lifetime

Four 6-cm plates of WT and Fto^{-/-} mESCs were seeded and controlled to afford the same number of cells, respectively. After 48 h, actinomycin D was added to 5 μg/mL at 6 h and 0 h before trypsinization and collection. The entire nuclear fraction was separated as mentioned in the cell fractionation section and total RNA was purified by TRIzol reagent (Thermo Scientific). External RNA Control Consortium (ERCC) RNA spike-in control (Ambion) was added proportional to total nuclear RNA to each sample before rRNA depletion using the RiboMinus Eukaryote kit (Thermo Scientific). Library preparation was performed using the SMARTer Stranded Total RNA-Seq Kit (Takara) v2 following the manufacturer's protocols. Sequencing was carried out at the University of Chicago Genomics Facility on an Illumina NovaSeq 6000 machine in paired-end mode with 100 base pairs (bp) per read (Data SI).

### RNA-Seq for Nascent RNA Synthesis

Six 6-cm plates of WT and Fto^{-/-} mESCs were seeded and controlled to afford the same number of cells, respectively. After 48 h, 5-Ethynyl Uridine (EU) was added to 0.5 mM at 60 min, 30 min, and 10 min before trypsinization and collection. Total RNA was purified by TRIzol reagents (Thermo Scientific) and nascent RNA was captured using the Click-iT Nascent RNA Capture Kit (Invitrogen) following the manufacturer's protocols. ERCC RNA spike-in control (Ambion) was added proportional to total RNA to each sample before rRNA depletion using the RiboMinus Eukaryote kit (Thermo Scientific). Library preparation was performed using the SMARTer Stranded Total RNA-Seq Kit (Takara) v2 following the manufacturer's protocols. Sequencing was carried out at the University of Chicago Genomics Facility on an Illumina NovaSeq 6000 machine in paired-end mode with 100 base pairs (bp) per read (Data S1).

Chromatin-Associated RNA (caRNA) m⁶a-Seq

1 μL 1:1000 diluted m⁶A spike-in from the EpiMark N⁶-Methyladenosine Enrichment Kit (NEB) was added as spike-in to 1 μg non-ribosomal RNA isolated from the chromatin-associated fraction. RNA was adjusted to 10 ng/μl in 100 μl and fragmented using a BioRuptor ultrasonicator (Diagenode) with 30 s on/off for 30 cycles. 5% of the fragmented RNA was saved as input. m⁶A-IP was performed using the EpiMark N⁶-Methyladenosine Enrichment Kit (NEB) following the manufacturer's protocols. Library preparation was performed using the SMARTer Stranded Total RNA-Seq Kit v2-Pico Input Mammalian (TaKaRa) following the manufacturer's protocols. Sequencing was carried out at the University of Chicago Genomics Facility on an Illumina NovaSeq 6000 machine in paired-end mode with 100 base pairs (bp) per read (Data S1).

Polyadenylated RNA m⁶a-Seq

1 μg polyadenylated RNA was adjusted to 10 ng/μl in 100 μl and fragmented using a BioRuptor ultrasonicator (Diagenode) with 30 s on/off for 30 cycles. 5% of the fragmented RNA was saved as input. m⁶A-IP was performed using the EpiMark N⁶-Methyladenosine Enrichment Kit (NEB) following the manufacturer's protocols. Library preparation was performed using the TruSeq Stranded mRNA Library Prep Kit (Illumina) following the manufacturer's protocols. Sequencing was carried out at the University of Chicago Genomics Facility on an Illumina HiSeq 2000 machine in single-end read mode with 50 bp per read (Data S1).

Low-Input Total RNA-Seq and qPCR for Oocyte and Embryo Samples

Embryos at GV, MII, and morula stages were transferred to 500 μl Trizol Reagent and then mixed well with 100 μl chloroform added. The mixture was transferred to a Qiagen MaXtract High Density tube (129046). After centrifugation, the upper and aqueous phase was removed. RNA was precipitated by adding an equal volume of isopropanol and washing with 75% ethanol. Then purified RNA was analyzed by RT-qPCR or subjected to library generation using the SMARTer Stranded Total RNA-Seq Kit (Takara Bio) following the manufacturer's protocol. Libraries were sequenced at Berry Genomics Corporation, Beijing, China or Nanjing Jiangbei New Area Biopharmaceutical Public Service Platform Co., Ltd on an Illumina NovaSeq 6000 platform in paired-end read mode with 150 bp per read (Data S1).

### ChIP-qPCR and ChIP-Seq

mESCs or Mel624 cells were cross-linked by adding 1% formaldehyde directly to the media for 10 min at RT. Cross-linking was stopped by adding glycine to a final concentration of 0.125 M and incubating for 5 min at RT. The media was removed and the cells were washed twice with ice-cold PBS. Chromatin immunoprecipitation was performed using the iDeal ChIP-seq kit for Histone marks (Diagenode) and the iDeal ChIP-seq kit for Transcription Factors (Diagenode), respectively, following the manufacturer's protocols. Spike-in antibody and chromatin (Active Motif) were added before immunoprecipitation. The precipitated DNA samples were either analyzed by qPCR or prepared for sequencing. Library preparation was performed using the NEBNext® Ultra™ II DNA Library Prep Kit for Illumina (New England Biolabs) following the manufacturer's protocols. Sequencing was carried out at the University of Chicago Genomics Facility on an Illumina NovaSeq 6000 machine in paired-end mode with 100 bp per read for mESCs and Illumina HiSeq 2000 machine in single-end read mode with 50 bp per read for Mel624 cells, respectively (Data S1).

### ATAC-Seq

ATAC-seq for mESCs or Mel624 cells was performed following a published protocol (42). In brief, 5×10⁶ cells were harvested and washed once with PBS buffer. The cell pellet was resuspended in 50 μL of cold lysis buffer and centrifuged at 4° C. at 500 g. Then the transposition reaction and purification were conducted. The transposed DNA fragments were amplified through PCR. Library preparation was performed using Nextera DNA Flex Library Prep Kit following the manufacturer's protocols. Sequencing was carried out at the University of Chicago Genomics Facility on an Illumina HiSeq 2000 machine in single-end read mode with 50 bp per read (Data S1).

m⁶A-Seq Data Analysis

Raw reads were trimmed by Trimmomatic (43) to remove low-quality bases and adapters, then aligned to the mouse genome (mm10), together with spike-in genomes including m⁶A and non-m⁶A spike-in from the NEB EpiMark N⁶-Methyladenosine Enrichment Kit using HISAT2 (version 2.1.0) (44) with ‘--rna-strandness RF-k 30’ parameters. Mapped reads were separated by strands with SAMtools (version 1.9) (45), using “samtools view-f 83 (and 163)” for forward strand and “samtools view-f 99 (and 147)” for reverse strand. m⁶A peaks on each strand were called using MACS2 (46) with the parameter ‘--nomodel’ separately. Significant peaks with q<0.01 identified by MACS2 (46) were considered. Peaks overlapped in at least two IP samples were merged using mergePeaks in Homer (http://homer.ucsd.edu/homer/ngs/peaks.html) and used for the subsequent analysis. The candidate peaks were assigned to the nearest genes by annotatePeaks in Homer (http://homer.ucsd.edu/homer/ngs/peaks.html) using the annotation file of version M20 (gtf format from GENCODE database, https://www.gencodegenes.org/). The numerical count of reads in m⁶A peaks was calculated by featureCounts with parameters “--minOverlap 30--fraction” (47) and then normalized by the number of reads mapped to m⁶A modified spike-in to represent the overall m⁶A level. Differential peaks were identified using QNB requiring p value<0.05 (48).

Quantification of m⁶A Levels of Chromatin-Associated RNA (caRNA)

Chromatin-associated regulatory RNAs (carRNAs), including enhancer RNA (eRNA), promoter-associated RNA (paRNA), and repeats RNA were defined in previous work (22) with slight modifications as below. Annotation of repeat RNAs (including divergence information of LINE1 subfamily) was downloaded from RepeatMasker (http://www.repeatmasker.org/) and the perl tool “one code to find them all” (49) was used to reconstruct full-length copies as described in a previous study (50). Read counts were quantified by featureCounts with parameters “--minOverlap 30--fraction” (47) and then normalized to per million total aligned reads to calculate counts per million (CPM). carRNAs with at least 10 reads in at least two input samples were kept for further analysis. The m⁶A ratio of the m⁶A modified spike-in from the NEB EpiMark N⁶-Methyladenosine Enrichment Kit (rₛₚᵢₖₑ₋ᵢₙ) was calculated as (CPM_{IP}+0.01)/(CPM_{Input}+0.01). The m⁶A normalization factor (nf) for each sample was defined as rₛₚᵢₖₑ₋ᵢₙ divided by average rₛₚᵢₖₑ₋ᵢₙ of all WT samples and was used to represent the overall m⁶A level of each sample. Then the m⁶A level for every carRNA and peak was calculated as (CPM_{IP}+0.01)/(CPM_{Input}+0.01)*nf. The m⁶A-labeled region of carRNAs and chromatin-associated mRNA (ca-mRNA, including both exonic and intronic regions) was defined as the region with an average m⁶A level larger than 2 in either WT or Fto KO samples. To identify the differentially m⁶A methylated carRNAs and ca-mRNAs, the inventors applied QNB (48) with nf as a size factor. The m⁶A-labeled regions with p<0.01 determined by QNB were identified as differential m⁶A regions, and the inventors further required the m⁶A changes (Fto KO/WT)>1.5-fold (|log₂FC|>0.58) in the subsequent analysis. The Pearson correlation coefficient of m⁶A level on m⁶A labeled peaks was used to assess the reproducibility between the biological replicates. IntersectBed from BEDTools (51) was used to assign the m⁶A peaks (i.e. peak.bed) to carRNAs or genes (i.e. regions.bed) with the parameters “-a peak.bed-b regions.bed-wao-s-f 0.5”.

caRNA RNA-Seq Data Analysis

Raw reads were trimmed by Trimmomatic (43) to remove low-quality bases and adapters, then aligned to the mouse genome (mm10) using HISAT2 (version 2.1.0) (44) with ‘--rna-strandness RF-k 30--seed 0’ parameters. Annotation files, version M20 in gtf format for mouse, were downloaded from GENCODE database (https://www.gencodegenes.org/). For carRNAs and ca-mRNAs, read counts were calculated by featureCounts (47). The differentially expressed carRNAs (p value<0.05) and ca-mRNAs (p.adj<0.1) were identified using the R package DESeq2 (52). For the repeats family, read counts were quantified by featureCounts (47) and differential expression analysis was performed using normalization methods in edgeR (53) combined with the voom transformation method in limma (54). P-values were determined using limma (54) and adjusted with the Benjamini-Hochber correction. Finally, differentially expressed carRNAs were identified with p.adj<0.01.

### Low-Input Total RNA-Seq Data Analysis

Raw reads were trimmed by Trimmomatic (43) to remove low-quality bases and adapters, then aligned to the mouse genome (mm10), together with ERCC RNA spike-in control (Thermo Fisher Scientific) using HISAT2 (version 2.1.0) (44) with ‘--rna-strandness RF-k 30--seed 0’ parameters. Annotation file, version M20 in gtf format for mouse, were downloaded from GENCODE database (https://www.gencodegenes.org/). For repeat RNAs, read counts were calculated by featureCounts (47) and the differentially expressed repeat RNAs were identified using the R package DESeq2 (52) with p value<0.05. The differential expressed genes were identified by DEseq2 with p.adj<0.01. For the repeat RNA family, reads counts were quantified by featureCounts (47), differential expression analysis was performed using the normalization method in edgeR combined with the voom transformation method in limma (54), and p-values were computed using limma and adjusted with the Benjamini-Hochberg correction.

### Polyadenylated RNA-Seq Data Analysis

Raw reads were trimmed by Trimmomatic (43) to remove low-quality bases and adapters, then aligned to the mouse genome (mm10) using HISAT2 (version 2.1.0) (44) with ‘--rna-strandness RF-k 5’ parameters. Annotation files, version M20 in gtf format for mouse, were downloaded from GENCODE database (https://www.gencodegenes.org/). Read counts on genes were calculated by featureCounts (47) and differentially expressed genes were called by DESeq2 with p.adj<0.1.

### Nuclear RNA Lifetime-Seq Data Analysis

Raw reads were trimmed by Trimmomatic (43) to remove low-quality bases and adapters, then aligned to the mouse genome (mm10), together with ERCC RNA spike-in control (Thermo Fisher Scientific) using HISAT2 (version 2.1.0) (44) with ‘--rna-strandness RF-k 30--seed 0’ parameters. Annotation file, version M20 in gtf format for mouse, were downloaded from GENCODE database (https://www.gencodegenes.org/). Reads on genes and carRNAs were counted by featureCounts (47) and then normalized to counts per million (CPM). CPM was converted to attomole by linear fitting of the RNA ERCC spike-in. Half-life of RNA was estimated using the mothed described in a previous paper (22). Specifically, as actinomycin D treatment results in transcription stalling, the change of RNA concentration at a given time (dC/dt) is proportional to the constant of RNA decay (K_{decay}) and the RNA concentration (C), leading to the following equation:

$$\frac{d C}{dt} = - {K}_{d e c a y} C$$

Thus, the RNA degradation rate K_{decay} was estimated by:

$$ln ( \frac{C}{{C}_{0}} ) = - {K}_{d e c a y} t$$

To calculate RNA half-life (t_{1/2}), when 50% of the RNA is decayed (that is

$$\frac{C}{{C}_{0}} = \frac{1}{2} ) ,$$

the equation was:

$$ln ( \frac{1}{2} ) = - {K}_{d e c a y} {t}_{\frac{1}{2}}$$

From which the inventors get:

$${t}_{\frac{1}{2}} = \frac{ln 2}{{K}_{d e c a y}}$$

### Nascent RNA-Seq Data Analysis

Raw reads were trimmed by Trimmomatic (43) to remove low-quality bases and adapters, then aligned to the mouse genome (mm10), together with ERCC RNA spike-in control (Thermo Fisher Scientific) using HISAT2 (version 2.1.0) (44) with ‘--rna-strandness RF-k 30’ parameters. Annotation files, version M20 in gtf format for mouse, were downloaded from GENCODE database (www.genecodegenes.org). Read counts on genes and carRNAs were counted by featureCounts (47) and then normalized to counts per million (CPM). CPM was converted to attomole by linear fitting of the RNA ERCC spike-in. RNA levels and timepoints of adding EU were fitted to a linear equation, and the slope was used to estimate the transcription rate of RNA as described in a previous paper (22).

Mettl3 KO and Ythde1 cKO caRNA m° a-Seq Data Analysis

Mettl3 KO and Ythdc1 cKO caRNA m⁶A-seq data was downloaded from NCBI's Gene Expression Omnibus (GEO) with GSE number GSE133600. Data analysis was conducted as described above.

Tissue-Specific Total RNA m⁶a-Seq Data Analysis

Tissue-specific total RNA m⁶A-seq data were downloaded from Genome Sequence Archive (GSA) with CRA001315 for human tissues and cell lines and CRA001962 for mouse tissues. Data analysis was conducted as described above. Specifically, the overall m⁶A value of LINE1 RNA was calculated as CPM_{IP}/CPM_{Input} of LINE1 RNA and the overall m⁶A value of repeat RNAs was calculated as CPM_{IP}/CPM_{Input} of all annotated repeat RNAs.

### ChIP-Seq Data Analysis

For mESCs, reads were first trimmed by Trim_Galore (https://www.bioinformatics.babraham.ac.uk/projects/trim_galore/) to remove adaptor sequences and low-quality nucleotides. Clean reads were then aligned to the mouse (mm10) and drosophila (dm6, for the spike-in histone) genomes using bowtie2 (55) with the parameter options ‘--very-sensitive--no-unal--end-to-end--no-mixed-X 2000’. The multi-mapped reads were kept for repeats analysis, but only the best alignment was reported for those aligned reads. The enriched peaks were called by MACS2 (46) with default parameters, and the common peaks between biological replicates were merged with mergePeaks in Homer (http://homer.ucsd.edu/homer/ngs/peaks.html) and used for further analysis. Bigwig files were generated by the deepTools (56) and normalized with RPKM after the library size normalized by the reads mapped on the drosophila genome (spike-in). The coverage of select regions was calculated by deepTools::computeMatrix method. For Mel624 cells, reads were aligned to human (hg19) reference genomes by Bowtie2 (55) with only uniquely mapped reads retained for downstream analysis. Other settings are same to those in mESCs.

### ATAC-Seq Data Analysis

Single-end sequencing reads were first trimmed by Trim_Galore (https://www.bioinformatics.babraham.ac.uk/projects/trim_galore/) to remove potential adaptor sequences and low-quality nucleotides. Clean reads were then aligned to reference genomes (mm9 and hg19 for mouse and human, respectively) by Bowtie2 (55), with only uniquely mapped reads retained for downstream analysis. Reads aligned to the mitochondrial genome were removed, together with PCR duplicated reads. ATAC-seq peaks were identified by MACS2 (46). To identify differentially opened ATAC-seq peaks, all peaks were merged into a united peak set by Homer (http://homer.ucsd.edu/homer/ngs/peaks.html). Reads were counted on peaks by FeatureCounts (47), and DESeq2 (52) was employed to perform statistical analysis. To identify differentially genomic regions, the inventors defined 1 kb sliding windows with ATAC-seq signals showing decreased intensity as gained-closed regions (57), and diffReps (58) was used with the default window size of 1 kb and step size of 100 bp. Functional enrichment analysis of differentially opened regions was performed by GREAT (59). Heatmaps showing overlap between ChIP-seq and ATAC-seq were generated with deepTools (56).

### Bioinformatic Settings to Differentiate LINE1 Subfamilies

The inventors acknowledge that given the repetitive nature of LINE1 and other repeats, there are limitations to accurately assigning reads if they are derived from the highly repetitive loci or regions with consensus sequences, even though the inventors carefully considered the strategy for multiple alignments at the mapping and read count calculation steps.

Our strategy to address this issue involved several steps. The inventors equally assigned these multiply aligned reads to different LINE1 subfamilies with consensus sequences, which may cause minor bias when calculating the read counts on these subfamilies. Raw reads were trimmed by Trimmomatic (43) to remove low-quality bases and adapters, then aligned to the mouse genome (mm10) using HISAT2 (version 2.1.0) (44) with ‘--rna-strandness RF-k 30--seed 0’ parameters. Annotation of LINE1 subfamily was downloaded from RepeatMasker (http://www.repeatmasker.org/) and the perl tool “one code to find them all” (49) was used to reconstruct full-length copies as described in a previous study (50). Then read counts on the LINE1 subfamilies were quantified by featureCounts with parameters “--minOverlap 30--fraction” to allow each alignment from a multiple-mapping read to carry a fractional count of 1/x, instead of 1, where x is the total number of alignments reported for the same read. The differentially expressed LINE1 subfamilies were determined using normalization methods in edgeR (53) combined with the voom transformation method in limma (54). P-values were computed using limma (54) and adjusted with the Benjamini-Hochberg correction. Notably, the inventors observed consistent expression changes in significantly downregulated young LINE1 subfamilies after separating multiply and uniquely mapped reads, indicating the stability of the bioinformatic analysis on these LINE1 subfamilies.

### Antibodies

The antibodies used in this study are summarized below: mouse monoclonal anti-FTO antibody (Abcam, ab92821); rabbit polyclonal anti-H3K27Ac antibody (Abcam, ab4729, used only in the ChIP-seq for Mel624 cells); rabbit monoclonal anti-H3K27Ac antibody (Cell Signaling, #8173S); rabbit monoclonal anti-H3K4Mel antibody (Cell Signaling, #5326S); rabbit monoclonal anti-H3K4Me3 antibody (Cell Signaling, #9751S); rabbit polyclonal anti-H3K9Me3 antibody (Active Motif, 39191); rabbit polyclonal anti-H4K20Me3 antibody (Abcam, ab9053) rabbit polyclonal anti-H3K27Me3 antibody (Cell Signaling, #9733S); mouse monoclonal anti-YY1 antibody (H-10) (Santa Cruz, sc-7341); rabbit monoclonal recombinant anti-EP300 antibody (Abcam, ab275378); rabbit polyclonal anti-METTL3 antibody (Bethyl, #A301-567A); rabbit polyclonal anti-YTHDC1 antibody (Abcam, ab122340); mouse monoclonal anti-α-Tubulin antibody (MilliporeSigma, F2168, for MII imaging); rabbit monoclonal anti-β-Tubulin (9F3) (Cell Signaling, 2128, for Western blot); rabbit monoclonal anti-Histone H3 antibody (Cell Signaling, 4499); rabbit monoclonal anti-GAPDH antibody (Cell Signaling, 5174); mouse monoclonal anti-U1 snRNP 70 antibody (Santa Cruz, sc-390988).

All of the methods disclosed and claimed herein can be made and executed without undue experimentation in light of the present disclosure. While the compositions and methods of this invention have been described in terms of preferred aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. I. A. Roundtree, M. E. Evans, T. Pan, C. He, Dynamic RNA modifications in gene expression regulation. Cell 169, 1187-1200 (2017).
2. M. Frye, B. T. Harada, M. Behm, C. He, RNA modifications modulate gene expression during development. Science 361, 1346-1349 (2018).
3. H. Shi, J. Wei, C. He, Where, when, and how: context-dependent functions of RNA methylation writers, readers, and erasers. Mol Cell 74, 640-650 (2019).
4. G. Jia et al., N⁶-methyladenosine in nuclear RNA is a major substrate of the obesity-associated FTO. Nat Chem Biol 7, 885-887 (2011).
5. F. van der Hoeven et al., Programmed cell death is affected in the novel mouse mutant Fused toes (Ft). Development 120, 2601-2607 (1994).
6. J. Fischer et al., Inactivation of the Fto gene protects from obesity. Nature 458, 894-898 (2009).
7. Z. Li et al., FTO plays an oncogenic role in acute myeloid leukemia as a N⁶-methyladenosine RNA demethylase. Cancer Cell 31, 127-141 (2017).
8. R. Su et al., R-2HG exhibits anti-tumor activity by targeting FTO/m⁶A/MYC/CEBPA signaling. Cell 172, 90-105.e123 (2018).
9. Y. Huang et al., Small-molecule targeting of oncogenic FTO demethylase in acute myeloid leukemia. Cancer Cell 35, 677-691.e610 (2019).
10. R. Su et al., Targeting FTO suppresses cancer stem cell maintenance and immune evasion. Cancer Cell 38, 79-96.el1 (2020).
11. Y. Qing et al., R-2-hydroxyglutarate attenuates aerobic glycolysis in leukemia by targeting the FTO/m A/PFKP/LDHB axis. Mol Cell 81, 922-939.e929 (2021).
12. Y. Liu et al., Tumors exploit FTO-mediated regulation of glycolytic metabolism to evade immune surveillance. Cell Metab 33, 1221-1233.e1211 (2021).
13. J. Liu et al., Landscape and regulation of m⁶A and m⁶Aₘ methylome across human and mouse tissues. Mol Cell 77, 426-440.e426 (2020).
14. J. Mauer et al., Reversible methylation of m⁶Aₘ in the 5′ cap controls mRNA stability. Nature 541, 371-375 (2017).
15. J. Mauer et al., FTO controls reversible m⁶Aₘ RNA methylation during snRNA biogenesis. Nat Chem Biol 15, 340-347 (2019).
16. J. Wei et al., Differential m⁶A, m⁶Aₘ, and m⁶A demethylation mediated by FTO in the cell nucleus and cytoplasm. Mol Cell 71, 973-985.e975 (2018).
17. K. Boulias et al., Identification of the m⁶Aₘ methyltransferase PCIF1 reveals the location and functions of m⁶Aₘ in the transcriptome. Mol Cell 75, 631-643.e638 (2019).
18. E. Sendinc et al., PCIF1 catalyzes m⁶Aₘ mRNA methylation to regulate gene expression. Mol Cell 75, 620-630.e629 (2019).
19. S. Akichika et al., Cap-specific terminal N⁶-methylation of RNA by an RNA polymerase II-associated methyltransferase. Science 363, eaav0080 (2019).
20. R. R. Pandey et al., The mammalian cap-specific m⁶Aₘ RNA methyltransferase PCIF1 regulates transcript levels in mouse tissues. Cell Rep 32, 108038 (2020).
21. P. J. Batista et al., m⁶A RNA modification controls cell fate transition in mammalian embryonic stem cells. Cell Stem Cell 15, 707-719 (2014).
22. J. Liu et al., N⁶-methyladenosine of chromosome-associated regulatory RNA regulates chromatin state and transcription. Science 367, 580-586 (2020).
23. J. Liu et al., The RNA m⁶A reader YTHDC1 silences retrotransposons and guards ES cell identity. Nature 591, 322-326 (2021).
24. W. Xu et al., METTL3 regulates heterochromatin in mouse embryonic stem cells. Nature 591, 317-321 (2021).
25. T. Chelmicki et al., m⁶A RNA methylation regulates the fate of endogenous retroviruses. Nature 591, 312-316 (2021).
26. J. W. Jachowicz et al., LINE-1 activation after fertilization regulates global chromatin accessibility in the early mouse embryo. Nat Genet 49, 1502-1510 (2017).
27. M. Percharde et al., A LINE1-Nucleolin partnership regulates early development and ESC identity. Cell 174, 391-405.e319 (2018).
28. J. Y. Lu et al., Genomic repeats categorize genes with distinct functions for orchestrated regulation. Cell Rep 30, 3296-3311.e3295 (2020).
29. A. A. Sigova et al., Transcription factor trapping by RNA in gene regulatory elements. Science 350, 978-981 (2015).
30. D. A. Bose et al., RNA binding to CBP stimulates histone acetylation and transcription. Cell 168, 135-149.e122 (2017).
31. T. Gerken et al., The obesity-associated FTO gene encodes a 2-oxoglutarate-dependent nucleic acid demethylase. Science 318, 1469-1472 (2007).
32. S. Malki, G. W. van der Heijden, K. A. O'Donnell, S. L. Martin, A. Bortvin, A role for retrotransposon LINE-1 in fetal oocyte attrition in mice. Dev Cell 29, 521-533 (2014).
33. S. Seisenberger et al., The dynamics of genome-wide DNA methylation reprogramming in mouse primordial germ cells. Mol Cell 48, 849-862 (2012).
34 Y. Wang et al., N⁶-methyladenosine RNA modification regulates embryonic neural stem cell self-renewal through histone modifications. Nat Neurosci 21, 195-206 (2018).
35. D. Guallar et al., RNA-dependent chromatin targeting of TET2 for endogenous retrovirus control in pluripotent stem cells. Nat Genet 50, 443-451 (2018).
36. J. Wei, C. He, Chromatin and transcriptional regulation by reversible RNA methylation. Curr Opin Cell Biol 70, 109-115 (2021).
37. X. Gao et al., The fat mass and obesity associated gene FTO functions in the brain to regulate postnatal growth in mice. PLOS One 5, e14005 (2010).
38. L. Li et al., Fat mass and obesity-associated (FTO) protein regulates adult neurogenesis. Hum Mol Genet 26, 2398-2411 (2017).
39. O. O. Abudayyeh et al., RNA targeting with CRISPR-Cas13. Nature 550, 280-284 (2017).
40. J. Wuarin, U. Schibler, Physical isolation of nascent RNA chains transcribed by RNA polymerase II: evidence for cotranscriptional splicing. Mol Cell Biol 14, 7219-7225 (1994).
41. L. A. Sanz, F. Chedin, High-resolution, strand-specific R-loop mapping via S9.6-based DNA-RNA immunoprecipitation and high-throughput sequencing. Nat Protoc 14, 1734-1755 (2019).
42. J. D. Buenrostro, P. G. Giresi, L. C. Zaba, H. Y. Chang, W. J. Greenleaf, Transposition of native chromatin for fast and sensitive epigenomic profiling of open chromatin, DNA-binding proteins and nucleosome position. Nat Methods 10, 1213-1218 (2013).
43. A. M. Bolger, M. Lohse, B. Usadel, Trimmomatic: a flexible trimmer for Illumina sequence data. Bioinformatics 30, 2114-2120 (2014).
44. D. Kim, B. Langmead, S. L. Salzberg, HISAT: a fast spliced aligner with low memory requirements. Nat Methods 12, 357-360 (2015).
45. H. Li et al., The Sequence Alignment/Map format and SAMtools. Bioinformatics 25, 2078-2079 (2009).
46. Y. Zhang et al., Model-based analysis of ChIP-Seq (MACS). Genome Biol 9, R137 (2008).
47. Y. Liao, G. K. Smyth, W. Shi, featureCounts: an efficient general purpose program for assigning sequence reads to genomic features. Bioinformatics 30, 923-930 (2014).
48. L. Liu, S. W. Zhang, Y. Huang, J. Meng, QNB: differential RNA methylation analysis for count-based small-sample sequencing data with a quad-negative binomial model. BMC Bioinformatics 18, 387 (2017).
49. M. Bailly-Bechet, A. Haudry, E. Lerat, “One code to find them all”: a perl tool to conveniently parse RepeatMasker output files. Mob DNA 5, 13 (2014).
50. H. M. Rowe et al., KAPI controls endogenous retroviruses in embryonic stem cells. Nature 463, 237-240 (2010).
51. A. R. Quinlan, I. M. Hall, BEDTools: a flexible suite of utilities for comparing genomic features. Bioinformatics 26, 841-842 (2010).
52. M. I. Love, W. Huber, S. Anders, Moderated estimation of fold change and dispersion for RNA-seq data with DESeq2. Genome Biol 15, 550 (2014).
53. M. D. Robinson, D. J. McCarthy, G. K. Smyth, edgeR: a Bioconductor package for differential expression analysis of digital gene expression data. Bioinformatics 26, 139-140 (2010).
54. M. E. Ritchie et al., limma powers differential expression analyses for RNA-sequencing and microarray studies. Nucleic Acids Res 43, e47 (2015).
55. B. Langmead, S. L. Salzberg, Fast gapped-read alignment with Bowtie 2. Nat Methods 9, 357-359 (2012).
56. F. Ramirez et al., deepTools2: a next generation web server for deep-sequencing data analysis. Nucleic Acids Res 44, W160-165 (2016).
57. F. Yan, D. R. Powell, D. J. Curtis, N. C. Wong, From reads to insight: a hitchhiker's guide to ATAC-seq data analysis. Genome Biol 21, 22 (2020).
58. L. Shen et al., diffReps: detecting differential chromatin modification sites from ChIP-seq data with biological replicates. PLOS One 8, e65598 (2013).
59. C. Y. McLean et al., GREAT improves functional interpretation of cis-regulatory regions. Nat Biotechnol 28, 495-501 (2010).
60. S. Relier et al., FTO-mediated cytoplasmic m⁶Aₘ demethylation adjusts stem-like properties in colorectal cancer cell. Nat Commun 12, 1716 (2021).
61. H. Chen et al., METTL4 is an snRNA m⁶Aₘ methyltransferase that regulates RNA splicing. Cell Res 30, 544-547 (2020).
62. S. M. Kweon et al., An adversarial DNA N⁶-methyladenine-sensor network preserves polycomb silencing. Mol Cell 74, 1138-1147.e1136 (2019).
63. S. Yang et al., m (6) A mRNA demethylase FTO regulates melanoma tumorigenicity and response to anti-PD-1 blockade. Nat Commun 10, 2782 (2019).
64. H. Huang et al., Recognition of RNA N⁶-methyladenosine by IGF2BP proteins enhances mRNA stability and translation. Nat Cell Biol 20, 285-295 (2018).
65. X. Wang et al., N⁶-methyladenosine-dependent regulation of messenger RNA stability. Nature 505, 117-120 (2014).
66. X. Wang et al., N⁶-methyladenosine modulates messenger RNA translation efficiency. Cell 161, 1388-1399 (2015).
67. S. Y. Hwang et al., L1 retrotransposons exploit RNA m⁶A modification as an evolutionary driving force. Nat Commun 12, 880 (2021).
68. J. Wu et al., The landscape of accessible chromatin in mammalian preimplantation embryos. Nature 534, 652-657 (2016).
69. K. Adachi et al., Esrrb unlocks silenced enhancers for reprogramming to naive pluripotency. Cell Stem Cell 23, 266-275.e266 (2018).
70. B. Ma et al., Molecular characteristics of early-stage female germ cells revealed by RNA sequencing of low-input cells and analysis of genome-wide DNA methylation. DNA Res 26, 105-117 (2019).

What is claimed is:

1. A method of increasing zygote implantation in an animal comprising implanting one or more fertilized cells into the reproductive tract of the animal, wherein prior to the implanting of the fertilized cells, exogenous fat mass and obesity-associated protein (FTO) and/or a nucleic acid molecule encoding FTO capable of transiently overexpressing exogenous FTO is introduced to the fertilized cells.

2. The method of claim 1, wherein the fertilized cells are implanted into the uterus or uterine lining of the animal.

3. The method of claim 1 or 2, wherein the fertilized cells are in a zygote stage of development when the nucleic acid and/or protein is introduced.

4. The method of any one of claims 1-3, wherein the implanting occurs when the fertilized cells are in a blastocyst stage of development.

5. The method of any one of claims 1-4, wherein the fertilized cells are contacted with the nucleic acid by injecting the nucleic acid into the fertilized cells.

6. The method of any one of claims 1-5, wherein the exogenous FTO is undetectable in the fertilized cells when the fertilized cells are in an embryonic stage of development.

7. The method of any one of claims 1-6, wherein the fertilized cells are fertilized in vitro.

8. The method of any one of claims 1-6, wherein the fertilized cells are produced from natural mating or artificial insemination.

9. The method of any one of claims 1-8, wherein the fertilized cells are collected from an animal through a non-surgical or a surgical technique.

10. The method of any one of claims 1-9, wherein the animal is a livestock animal.

11. The method of any one of claims 1-10, wherein the animal is a cow.

12. The method of any one of claims 1-10, wherein the animal is a pig.

13. The method of any one of claims 1-9, wherein the animal is a companion animal.

14. The method of claim 13, wherein the companion animal is a dog or cat.

15. The method of any one of claims 1-9 wherein the animal is a research animal or animal used for xenotransplantation.

16. The method of claim 15, wherein the research animal is a mouse or rat.

17. The method of any one of claims 1-9, wherein the animal is a human.

18. The method of any one of claims 1-9, wherein the animal is an endangered animal.

19. The method of any one of claims 1-17, wherein the animal is a different species than the species of the fertilized cell.

20. The method of any one of claims 1-19, wherein the nucleic acid molecule encoding FTO is an mRNA molecule.

21. The method of any one of claims 1-19, wherein the nucleic acid molecule encoding FTO is a DNA molecule.

22. The method of claim 21, wherein the DNA molecule is an expression construct.

23. The method of any one of claims 1-22, wherein a viral vector is used and the nucleic acid molecule is introduced to the fertilized cell through the use of a viral vector.

24. The method of any one of claims 1-22, wherein the nucleic acid molecule is introduced into the fertilized cell by transfection.

25. The method of any one of claims 1-24, wherein the method further comprises monitoring the animal for implantation of the fertilized cells.

26. The method of any one of claims 1-25, wherein the method further comprises monitoring the animal for pregnancy.

27. The method of any one of claims 1-26, wherein the method further comprises measuring one or more biomarkers of pregnancy in the animal.

28. The method of claim 27, wherein the biomarker of pregnancy comprises chorionic gonadotropin.

29. The method of any one of claims 1-28, wherein the method further comprises measuring a demethylation status in the fertilized cells.

30. The method of any one of claims 1-29, wherein the method further comprises culturing and/or incubating the cells in a media and/or buffer.

31. A method of increasing birth weight of offspring of an animal comprising implanting one or more fertilized cells into the reproductive tract of the animal, wherein prior to the implanting of the fertilized cells, exogenous fat mass and obesity-associated protein (FTO) and/or a nucleic acid molecule encoding FTO capable of transiently overexpressing exogenous FTO is introduced to the fertilized cells.

32. The method of claim 31, wherein the fertilized cells are implanted into the uterus or uterine lining of the animal.

33. The method of claim 31 or 32, wherein the fertilized cells are in a zygote stage of development when the nucleic acid and/or protein is introduced.

34. The method of any one of claims 31-33, wherein the implanting occurs when the fertilized cells are in a blastocyst stage of development.

35. The method of any one of claims 31-34, wherein the fertilized cells are contacted with the nucleic acid by injecting the nucleic acid into the fertilized cells.

36. The method of any one of claims 31-35, wherein the exogenous FTO is undetectable in the fertilized cells when the fertilized cells are in an embryonic stage of development.

37. The method of any one of claims 31-36, wherein the fertilized cells are fertilized in vitro.

38. The method of any one of claims 31-36, wherein the fertilized cells are produced from natural mating or artificial insemination.

39. The method of any one of claims 31-38, wherein the fertilized cells are collected from an animal through a non-surgical or a surgical technique.

40. The method of any one of claims 31-39, wherein the animal is a livestock animal.

41. The method of any one of claims 31-40, wherein the animal is a cow.

42. The method of any one of claims 31-40, wherein the animal is a pig.

43. The method of any one of claims 31-39, wherein the animal is a companion animal.

44. The method of claim 43, wherein the companion animal is a dog or cat.

45. The method of any one of claims 31-39 wherein the animal is a research animal or an animal for xenotransplantation products.

46. The method of claim 45, wherein the research animal is a mouse or rat.

47. The method of any one of claims 31-39, wherein the animal is a human.

48. The method of any one of claims 31-39, wherein the animal is an endangered animal.

49. The method of any one of claims 31-47, wherein the animal is a different species than the species of the fertilized cell.

50. The method of any one of claims 31-49, wherein the nucleic acid molecule encoding FTO is an mRNA molecule.

51. The method of any one of claims 31-49, wherein the nucleic acid molecule encoding FTO is a DNA molecule.

52. The method of claim 51, wherein the DNA molecule is an expression construct.

53. The method of any one of claims 31-52, wherein a viral vector is used and the nucleic acid molecule is introduced to the fertilized cell through the use of a viral vector.

54. The method of any one of claims 31-52, wherein the nucleic acid molecule is introduced into the fertilized cell by transfection.

55. The method of any one of claims 31-54, wherein the method further comprises monitoring the animal for implantation of the fertilized cells.

56. The method of any one of claims 31-55, wherein the method further comprises monitoring the animal for pregnancy.

57. The method of any one of claims 31-56, wherein the method further comprises measuring one or more biomarkers of pregnancy in the animal.

58. The method of claim 57, wherein the biomarker of pregnancy comprises chorionic gonadotropin.

59. The method of any one of claims 31-58, wherein the method further comprises measuring a demethylation status in the fertilized cells.

60. The method of any one of claims 31-59, wherein the method further comprises culturing and/or incubating the cells in a media and/or buffer.

61. A method of increasing birth weight of offspring of an animal comprising implanting one or more fertilized cells into the reproductive tract of the animal, wherein prior to the implanting of the fertilized cells, exogenous fat mass and obesity-associated protein (FTO) and/or a nucleic acid molecule encoding FTO capable of transiently overexpressing exogenous FTO is introduced to the fertilized cells.

62. The method of claim 61, wherein the fertilized cells are implanted into the uterus or uterine lining of the animal.

63. The method of claim 61 or 62, wherein the fertilized cells are in a zygote stage of development when the nucleic acid and/or protein is introduced.

64. The method of any one of claims 61-63, wherein the implanting occurs when the fertilized cells are in a blastocyst stage of development.

65. The method of any one of claims 61-64, wherein the fertilized cells are contacted with the nucleic acid by injecting the nucleic acid into the fertilized cells.

66. The method of any one of claims 61-65, wherein the exogenous FTO is undetectable in the fertilized cells when the fertilized cells are in an embryonic stage of development.

67. The method of any one of claims 61-66, wherein the fertilized cells are fertilized in vitro.

68. The method of any one of claims 61-66, wherein the fertilized cells are produced from natural mating or artificial insemination.

69. The method of any one of claims 61-68, wherein the fertilized cells are collected from an animal through a non-surgical or a surgical technique.

70. The method of any one of claims 61-69, wherein the animal is a livestock animal.

71. The method of any one of claims 61-70, wherein the animal is a cow.

72. The method of any one of claims 61-70, wherein the animal is a pig.

73. The method of any one of claims 61-69, wherein the animal is a companion animal.

74. The method of claim 73, wherein the companion animal is a dog or cat.

75. The method of any one of claims 61-69 wherein the animal is a research animal or an animal for xenotransplantation products.

76. The method of claim 75, wherein the research animal is a mouse or rat.

77. The method of any one of claims 61-69, wherein the animal is a human.

78. The method of any one of claims 61-69, wherein the animal is an endangered animal.

79. The method of any one of claims 61-77, wherein the animal is a different species than the species of the fertilized cell.

80. The method of any one of claims 61-79, wherein the nucleic acid molecule encoding FTO is an mRNA molecule.

81. The method of any one of claims 61-79, wherein the nucleic acid molecule encoding FTO is a DNA molecule.

82. The method of claim 81, wherein the DNA molecule is an expression construct.

83. The method of any one of claims 61-82, wherein a viral vector is used and the nucleic acid molecule is introduced to the fertilized cell through the use of a viral vector.

84. The method of any one of claims 61-82, wherein the nucleic acid molecule is introduced into the fertilized cell by transfection.

85. The method of any one of claims 61-84, wherein the method further comprises monitoring the animal for implantation of the fertilized cells.

86. The method of any one of claims 61-85, wherein the method further comprises monitoring the animal for pregnancy.

87. The method of any one of claims 61-86, wherein the method further comprises measuring one or more biomarkers of pregnancy in the animal.

88. The method of claim 87, wherein the biomarker of pregnancy comprises chorionic gonadotropin.

89. The method of any one of claims 61-88, wherein the method further comprises measuring a demethylation status in the fertilized cells.

90. The method of any one of claims 61-89, wherein the method further comprises culturing and/or incubating the cells in a media and/or buffer.

91. A fertilized cell comprising an exogenous FTO nucleic acid and/or an exogenous FTO protein.

92. The fertilized cell of claim 91, wherein the fertilized cell is in a zygote stage of development.

93. The fertilized cell of claim 91 or 92, wherein the fertilized cell is a human cell, a livestock animal cell, a companion animal cell, or a research animal cell.

94. The fertilized cell of any one of claims 92-93, wherein the fertilized cell is generated using the method of any one of claims 5-25.

95. A composition comprising the fertilized cell of any one of claims 91-94.

96. A composition comprising the fertilized cell of any one of claims 91-94 and one or more reagents used for in vitro fertilization.

97. A method for demethylating long-interspersed element-1 (LINE1) RNA in a cell, the method comprising providing to the cell an effective amount of fat mass and obesity-associated protein (FTO) and/or a nucleic acid molecule encoding FTO.

98. The method of claim 97, wherein the LINE1 RNA is chromatin-associated regulatory RNA.

99. The method of claim 97 or 98, wherein the method comprises providing a nucleic acid encoding for FTO.

100. The method of claim 97 or 98, wherein the method comprises providing FTO protein.

101. The method of any of claims 97-100, wherein the methylation is m⁶A methylation.

102. A method for modifying expression level of a gene in a cell, the method comprising providing to the cell an effective amount of FTO or a nucleic acid molecule encoding FTO.

103. The method of claim 102, wherein the method comprises providing a nucleic acid encoding for FTO.

104. The method of claim 102, wherein the method comprises providing FTO protein.

105. The method of any of claims 102-104, wherein the gene comprises a LINE1 element.

106. The method of any of claims 102-104, wherein the gene is a 2C gene.

107. A method for increasing chromatin accessibility in a cell, the method comprising providing to the cell an effective amount of FTO or a nucleic acid molecule encoding FTO.

108. The method of claim 107, wherein the amount is effective to decrease an amount of histone modifications in the cell.

109. The method of claim 107 or 108, wherein the method comprises providing a nucleic acid encoding for FTO.

110. The method of claim 107 or 108, wherein the method comprises providing FTO protein.

111. The method of any of claims 97-110, wherein the cell is a stem cell.

112. The method of claim 111, wherein the cell is an embryonic stem cell.

113. The method of claim 111, wherein the cell is an induced pluripotent stem cell.

114. The method of any of claims 97-113, wherein the cell is a mouse cell.

115. The method of any of claims 97-113, wherein the cell is a human cell.

116. The method of any of claims 97-115, wherein the cell is a cancer cell.

117. A method for modifying development of a germ cell, the method comprising providing to the cell an effective amount of FTO or a nucleic acid molecule encoding FTO.

118. The method of claim 117, wherein the amount is effective to increase chromatin accessibility in the cell.

119. The method of claim 117 or 118, wherein the method comprises providing a nucleic acid encoding for FTO.

120. The method of claim 117 or 118, wherein the method comprises providing FTO protein.

121. The method of any of claims 117-120, wherein the germ cell is an oocyte.

122. The method of any of claims 117-121, wherein the germ cell is a mouse cell.

123. The method of any one of claims 97-122, wherein the method further comprises measuring a methylation status in the cell.

124. The method of any one of claims 97-123, wherein the method further comprises culturing the cell.
